SEQUENCE LISTING (41 sequences)

SEQ ID NO: 1 - DNA, 681 bp, Prevotella copri
gctatcctcctttcgcctttcgagaacagcggtggatatggcaagctcgacaagctgcatatccccatcatcgaagctgccgactatatggagtcttcgccactgggcagggcggaatggatgaaattctacggcatgctctttggaaatgaagagggaaaaagtaacggaatatcgggttcttgcgaaccaaaggcagattcgctctttgcgaagatagaaaaggaatatctttcgcttaaggcgcaggcagccgggtatcggaagggcctctccatcctgaccgaaagaaaaacgggaaacgtatggtatgtgcctggtggtcagagtactatcggtattctgctgaaggatgccaacgcccgttatatcttcgaggacgatcagcacagcggaagtctggcgatgagtccggaacaaatcttggcgaagggaaagcaggtggatgtatgggcattcaagtatttcggtggtgctcctctgtcgcaggctcaactgcttcaggaatatgacggctacaaggctcttgctgccttcaaccggggtaatatctatcaggtggatacctctacggtaccttatttcgagcttacgagtttccatcccgaactgctgctcagagagttcatcatcctggcccatggcgagcggttcggcaaattgagattctataagaaatag

SEQ ID NO: 2 - DNA, 2841 bp, Prevotella copri
atgaattcagaagaaaagataaatgtatggggcgcacgggtccacaacctcaagaacatagatgtggagattccgcgtgattccctcaccgtgattaccggactctcgggttcgggcaagagttcactggccttcgacaccatctttgccgaaggtcagcgcagatatatcgaaacattctctgcctatgcccgcaacttcctgggtaatatggaacgcccggatgttgataagattacgggattgagtccggtcatcagcatagaacagaaaaccaccaacaagaatccccgctctaccgtgggcactaccaccgagatttacgactatctccgtctgctctatgcccgcgccggtactgcctacagctatcagagtggcgaggagatgatgaaatataccgaggaacaggtcatcgacatgattctcagcgattataagggcaaggctatcttcctgctcgcacccctcgtacgccagcgcaagggtcattaccgcgaactcttcgaaagtatgcgcagaaaggggtatctctacgtgcgactcgacggcaacatcctcgaaatcgttcccaatatgaaaaccgaccgctacaagaaccacaatatagaagctgtggtcgataaactggtggtgaaggaagaagatgaggaacgcatccgcaagagtgtggcaacggctatgaaacagggcgacggcatggtaatggttcttgagaaaggtgccaaggaggccaagacatactccaagcgtctcatggaccctgtaacgggtatcgcctaccaggatccagctcctaacatgttctccttcaactctcccgaaggtgcctgtccgcattgcaagggtctgggaaaggtgaaccagattgatatcaagaaggtaattcccgatgataaactcagcatccatgaaggtggcatcgctccgctcggcaaatacaagaaccagatgattttctggcagatagaatcattgctcagcaagtatgacctcaacctgaagacacctatctgcgagattccgggcgatgccatgcaggaaattctctatggctcgctcgaaaacgtgaagattgagaaggaaaaggtgcatacctctaccgactatttctgcgcctacgatggcatcatcgactacctgcagaaggtgatggaggatgacgagagtgcggcgggcaagaaatgggccgaccagtttatctctaccatcgaatgtcctgaatgccatggtttgcgactcaaaaaagaatcgctctctttcaagatctgggataagaatatatccgaagtagccagtctcgacatcgatgaactgcgcgattggctcgaagaagtggaacagcatctgccttcgatgaaagcgaaggtggctcacgaaatcatcaaggagttgcgctcacgcgttaccttcctcctcgatgtgggactcaattatctctcgctcaaccgccagtcggcttctctatcgggtggcgagagccagcgcatccgtctggctactcagattggtagccagctggtcaatgtactctatatcctcgatgagccgagcatcggtctgcaccagcgcgacaacgaacgtctgctcaacagtctgaaggaacttcgcgacctgggcaataccgttattgtggtagagcacgatgaagatatgatgcgggctgccgactggatagtagatatcggtccgaaggcgggacgcaagggaggtgaagttgtcttccagggcactccccaggagatgctcaagaccgataccatcaccgcccagtatctcaacggcaagatggctattgaggtgcctgccctccgcagagaaggcaacggcaagcatattaccattcacggagctacgggcaacaacctcaagggggtggatgttgattttccactcggcaaactcattgtcgtaacgggtgtcagcggctcaggcaaatctactctcatcaacgagactctccagcctatcctctcgcagcatttctaccgttcgctcaagaaaccgatgccatacgagagcatcgatggtatcgagaatatcgacaaggtagtgaatgtagaccagagtcctattggcagaacgcctcgcagcaatccagctacctacacaggcgtgttcagcgatatccgctcgctcttcgtaggacttcccgaagcaaagatccgcggttacaagccgggcagattctccttcaatgtgaaaggtggaagatgcgaggaatgtaagggtaacggatataagaccatcgagatgaacttcctgccggatgtctacgtaccttgtgaggtatgccacggcaaacgctacaaccgcgagacgctggaggtaagatacaagggaaagagcatcgccgatgttctcgacatgaccatcaaccaggcggtagatttcttcgagaatgttcctcagatactgcagaagatcaaggctttacagaatgtaggactcggatacatcaggttaggacagagttccactaccctctctggaggtgagagtcagcgcgtaaaactggctaccgaactctcgaagcgtgatacgggcaagacgctctatattctcgatgaaccgacaaccggtctacatttcgaagacatccgcatcctgatggatgtactccagaaactggtagaccgtggcaatacggtcattatcatcgagcacaatctcgatgtcatcaaactcgccgactggctcatcgacatgggtccggaaggtggacgaggcggcggtcagctcctctttgctggaacaccagaagaaatggtaaagcagcagaagggatatacctataagttcctcgcccctttactgaagaaatcagggaaacctgaataa

SEQ ID NO: 3 - DNA, 516 bp, Prevotella copri
atgagaaaagaatcaagagcaatggatagtctatgggcattggaagtaatgcacaaggctccgtatataactgtcagttttattgacgaagacggcaagccttacggtttacctctttcacttgcatcagatgatgatgtgaactggtattttcatggtgccttggaaggcaaaaaactggaggcaatcaaggctcatcctgaggtttgcctttcagccgtaacccgttgtgcgcctacggttggtccgaaggacggcagtttcaccctgcaattcaaatcagccattgcattcggcaaggcagaaatcgtgacagatgaagcagagaagattcatggtctccggctaatctgtgaacgtttccttcctcaacacatggatgctttcgaccagagcatcgcccgttccctttcacgcacggctgttgttcgcatcacgcttactgagccaccaaccggcaaacgcaagcagtatgataaggagggcgtggaaatgaaatatgggagaatggaataa

SEQ ID NO: 4 - DNA, 1032 bp, Prevotella copri
atgaaaaccattgttttaggggcaatcgccattatcatactgtttttcgccaacctggcatgggggagtgtaaacatcccgtggcaggacgtgggggcgattatttcgggttctcagaccgacgaaacctaccgctatattctgttggaatcgcgactgccggcggccattaccgccttgctttcgggcgcagccctcgccacgagcggcttgctcctgcagacagccttccgcaaccccttggcgggtcctgacgtattcggaataagcagcggagccggactggcggtagccatcgtgatgcttgcctttggcggcaatattgccctggatgatttaggggtaggttttttgggcgatgccggcaactatgccatcggcggtttcctggctgtcctcatcgcagcctttataggggctatggtggtgatgggtatcatcaccttcttttctgccattgtgcgcagccatacggtactgctcatcatcggactgatggtgggttatctggcaagcagcgccatctcattgctcaatttcttcagtacggcagagggcgtgaaatcgtatatggtttggggcatgggcagttttggcaatgtttcgagccagcagatgatgttcttcatcccgttggtcctcattgccctggctgcatctctgctccttgtaaaaccgctgaacgccatgctgttaggcgagcagtatgccgagaatctgggcttcaacatcaggcgcctgcgcatcgttctgctcatcatcaccggtcttcttacggcagtggttaccgccttctgcggtcccatcgcctttatcggtctggcaacgccgcatatcgcccgcctcatcatcggcacggagaatcatcgccgactgttgcccgttacgatgctgatgggtgcatccattgcccttctctgcaatctcttctgcacccttccgtcggatggaggcatcatcccactgaatgccgtcaccccgctgttcggcgcccccgtcatcatctatgttttggtgaaaaagcgcatctga

SEQ ID NO: 5 - DNA, 966 bp, Prevotella copri
attccaaaccgtcgtcagcgcatggtgtatggaggtgctattctgctttctgccaactgtggcggagctcttaccgttatcggaaatccggaaggactggtaatgtggaatatgggtgctgtaacggctacccattatttcctgtcgcttctgctgccatgtcttgctgcttggctcataccattggtgatgatgcagcgcatgctgccggaaagggtagagacagaatggattgctatgccataccgcggggatgatacccgcctcaatgtatggcagcgcttgctgatgctctttgtgggtatcggtggattgtggtttatccctacctttcataatattacgaaattgagtcctttcctcggtgcactctgcgtattaggtgtgctatggatagtgaacgagatattcaaccgtaaactcatgaatatggatgcgatggcagaacgtcgtactcctagagtcttccaatacggagtcgtccagatgattctcttcgtgatgggtatcatgcttgccatcggagtggtgaaggagaccggcgcttttgacgattttgctactcttctcaactctgtcggtatggatgataagcgtcctggcgtgctgttgcatggcgtcttggctggtatcatcagtactgtacttgacaactttgctacagccatgaattttttctcccttcatgatctggcaaatgtgaatgatccttcattcagcatgcttactgattatcaaaccaatggtatctactggcagatgattgcatattgcgtaatggctggtggtaacgttcttggtatcggtacaatcagcggtcttgccctgatgaagatggaacgtatgcacatggggtggtatttccgcaatattggctggaaagctttgatgggtggcgtcatcggacttgctatcctttggctctcccatatcctggtggctggtgccgcaaacctaattatttaa

SEQ ID NO: 6 - DNA, 354 bp, Prevotella copri
ttgcagattattgccaatgggttggctacagtttctacacatgaacttttcacttatatcgaaacttatattcagaaagaacaggtggaacgtattgttatcggaaaaccgatgcaacctaacgggcagcctagtgaaaacctggcaagagtagaaaacttctacaaccgttggcgcaaggctcatcctgaaattcctattgaatattatgacgagaggtttacatcagtcctggcacatagagccatgatagatggaggtgtaaagaagaaagtgagaaaagaaaacaaaggattggtagacgagataagtgctaccatcatattacaggattatttgcaatcaagaaaataa

SEQ ID NO: 7 - DNA, 1203 bp, Prevotella copri
atgaaaagaatgaaaaaaggattgcggctattggctgtggcaggactcatggtgttctcgctgagcagttttatgcccgctaaaaatgtattcacggcagccgaacagcagcaggtgagtattgcaacacccggtctctttgcccagagtcaggcatttaatgtagactttgagatattccgtgctaaggaatattccttcccgcttcctgtgggcaaagcaacccttctgaacaatcatgtgttgcgtatctctacctctaaaggcgatgctgtcaaggcgatgctcgaaggttatgtgcgactttctagaaagtcggaatctatgggtaatgtcatcgtggtgcgccacgattgcggtctggaaaccgtttatgccaacaatgccgagaacctggttaaggtaggacagcatgtggatgccggacagacgattgccattgtaggctctaaggagggtgaaacttattgtgacttctctatcatggtgaatggcggtaggttgaatccggaaactttcgtagagttgaaatcgcataagttgcgccgccagaccgtgcagttccgcaagcatggtgcccgtgtcatcgcttcggttatcggtggcaaggattcttctgtaggaagaaatgctgaggctgacaaggaggcatctggcaagaaaaagggtagaataggagatggaatgacgctcgatcctgatgaggtttatgatccgtttaccattaccaatactttcgagcttgatttggagaagatagaaaagacagcctgggcttatcctttgccgggtgctaaggtaatcagtccttatggaggcaagcgccgccattcgggagttgacctcaagacgtgcccgaatgatgagattgtggctgcctttgatggtaaggttgttgcatcaggaccttattttggctatggcaactgcatccgtatcaagcatgcttatggtttcgagacgctctacagccatcagagcaaaaacaaggtgaagaagggcgataaggtaaaggctggtcaggtaatcggcttgacgggccgtaccggtagagcgactacagagcacctccatttcgaggttagctttggtggcagacgactggacccagccatcatttatgaccacggcaagcatcaattgaagcctgtaacccttcatctaacgaagggtaggggcgtcaagagtgtaaagaaccgttag

SEQ ID NO: 8 - DNA, 957 bp, Prevotella copri
atgaaaacgaatagaaccaaaatactcagcaccacggcgctgatagccgtgctctgcatgcaactgttctggatgtggaactccttcgagatgaccgttcaccagatggggtttgaaacaccctggaacctggcaccggatgtcagggcacaggccctgctcgcagccttttacgaaagcaagttcaccgttctcacctctctcctcaccacggtggtcatcatcctgagcctcatcgaccagattaactacatcgatgagcaggaaagggtgcgcctgctgcgcgaggacttctcctatgcaatggttcacgacatgaagtcgccccttacctctatcatcatgggaaccaaatatctgcacagcggcgtactggagaagaagccggaaatgaaggagaaatatttctgtatcgtagaagacgaggcacagcatctgcttgctctcatcaaccgcctgcttaccatctcaaaactggaacatggtaaactgagtatccagaaggctgaaatagatctggaggcgatgatagaggatgtggtggataaatacaaggcgaaatcggcgaaaccgattcatatcaccaccctattcggagccacttcggcactggcagataaggaatatctgaaggaggctatcagtaatatagtagataacgccaccaaatactcgaaggaagaaatcaacattcagatttccacctccgagaatgacagaaatgtatatatcaagatatacgatgagggtatcggcatagccagaagtgagatgaaaacaatcttcaaccgctttgagcgtgctgctgagcacgaaagagacgcccggaagacccgtggcggcttcggtatcgggctgaactacgtgctgcaggtaatcaatgcccatggcggcaaagtaagcgtaaagagcgaaaaaggcaaatggtcggagtttaccatatcgctgccgaaataa

SEQ ID NO: 9 - DNA, 474 bp, Prevotella copri
agctgtcctaagctgactcgcggtaagtttgagtacgatttcggtgatgaggcaggttacactccattgctgccaatgtttactctgggtcatgatttcaagccagccaacattcatgccggtggtctgcgttatcatggtgcgggtatgattatctcacagcttctgaaggatggttacctgcatggtgtagatatccctcagctggagtcattcaaggcgggtatgctctttgcacagaccgagggtatcattcctgctccagagagctgtcatgcgattgcggccactatacgtgaggctctgaaggctaaggaagagggcaaggagaaggttatcctgttctgtctctcaggtcatggtcttatcgatatgccgtcttacgatagctttatcaatggcgatttgcacgattattctgtgagtgatgaagaaatccagcagttcctcaaggatgtgccaaaggttgactaa

SEQ ID NO: 10 - DNA, 954 bp, Prevotella copri
atgcgaacaaaacaaaataattcccatttttttttgtattattctcgatttgcagtatctttgcatccgcaaaatgtaataaaaatgaaagatatttgttgtattgggcacgtaacaaaggataaaatcgtgaccccgagcagtacagtttacatggctggcggcacctctttttattttgcctacgccatcaaccaattgccaaaggatgtaagtttctcgctcattacggcgatggatcctaccgagaaggaacctgttgaaaagatgctcaaggctggaatagacgtcaccttgaacccatcgcgcaatacggttttcttcgagaatatttatggcgataatcctaacgaccgtaagcagcgtgtgcttgccaaggcagatcctttcaccatccagcaactggagcatgtggaggcaaaggtttttcacctgggcagtttgctgagcgatgatttctcgccggaagttgttgcctttcttgccaagaagggaaaagtttccatcgatgtgcagggctatctacgcgaggtgagagacgagaaagtttacgccatcgactggaaggacaagctcgatgtgctcaaaaacacctattatctgaaggtgaatgagaccgaaatggagaccattacaggactgaaggacccgaaggaagccgccaagctgattcatgcctggggcgtagccgaagtgatcatcaccctgggtagcgagggctctctggtttatgtagatgatacgttctacgacattccagcctatcctcctcatgaagtagtagatgctaccggttgtggcgatacctattcggcaggctatctctacaagcgtctgcagggagccaatccggtagaagccggcaagtttgcggcagccatgtgtaccatcaaactggagcacaatggtccgttcaaccgcaccatcgaagatgtaatgaaaatcatcagataa

SEQ ID NO: 11 - DNA, 1797 bp, Prevotella copri
gtgccggttccagaactggacgatgagaatcatgtgctcttgagttcgctggatgttacggtgattcagcatggagctgagtttaacctcggcttgcacaagtatcagggcaacaactatagtccgatgggccacaagtacattcgtgagtttgattgtgataaagtgccaactaccctttatcgcgtaggtggtgttatcctgaaaaaggaagttgtattccagcattacgagaatcgcattctgattcgctatacgctggtagacggccattcggctacaacccttcgcttccgtccttttctggctttccgcagtgtccgtcagtttactcatgagaatgctaccgcatctcgtgattatgctgaggtagatcatggcatcaagacctgtatgtatgcaggttatcctgatctctatatgcagttctccaagaagaacgagtttaaattctgtccagattggtatcgtggcgtggaatatccaaaggagcaggagagaggttatgcttctaacgaagacctctatgttcctggctattttgaaatggatatcaagaaaggcgaaaccatcgtctttgctgcttctacgtcagaaatcaaggcggtcagcctgaagaagctcttcgacaaggaagtggatgagcgttcgcctcgtgacaatttcttccactgtctggtcaatgcggctcatcagttccatcgtcgtgaaaagaacgatgaccgttatatcctggcaggttatccttggttcaagtgcagagcccgcgatacatttatcgctcttccgggtctcaccctctctatcgaggaagatgactacttcgaactggtgatgaagactgccatgaagggatactacgagtttatggaaggcaagccggtcagcgttcgtattgctgagatagagcagcctgacgtgcctttgtgggctatctgggctttgcagcagtatgccaaggaaaccagcaaggaagcatgcttcaagaagtatggacagtttatcaaggatgtcatctcctttatccaggataacaagcatccgaacctgaagctcgaagagaacggattgctttatactgacggtaaggataaggctgtaacctggatgaactctacagccaatggcagaccagtggttccacgtacaggttatatcgtagagtttaatgcgctgtggtataacgccttgtgtttctgtgcttctcttgctgcaacagtaggcgaggaagacaaccagcagaaacttctggctcaggctgagaaaaccaagcaggcgttcctcgataccttcctcaatgaatatggctatctttacgattatgtagatggcaatatgatggactggagcgttcgtccgaacatgatatttgcagtggctttcgactattcaccattgtcgcaagaccagaagaagcaggtacttgatatctgtacacgcgaacttcttactcctaagggattgcgttcactctcgccaaagagcggtggatataatcctgtttatgtaggtccgcagacccagcgcgactatgcttaccatcagggtacggcatggccatggctcggcggcttctatatggaggcaagtcttaaactctataagcgtacccgtttgagctttatcgaacgccagatggtaggttatgaggacgaaatgtcttaccactgtctaggtaccatcagcgaactcttcgatggaaaccctccattcgcaggtcgtggtgccatctctttcgccatgaatgtggctgagattctgcgtgcgctcgagttacttgaaaaatatcaatattaa

SEQ ID NO: 12 - DNA, 750 bp, Prevotella copri
atcgagaagcatgcctttgatgccgagaacaacggatatatcgaggcattgacccgtgaatggaatccgattgcagacatgcgtctctctgataaggatgagaatggttcccgtacgatgaatacccatctgcatatcatcgagccatataccaatctttatcgtgtatggaagactccggaactggagaagagcatccgcaatctcctcgatatcttcaccgataagcttctgaacacagagacttatcatctcgacctcttcttcaatgacgagtgggagggcaagcgcaacatcgagagttacggtcatgatatcgaggcatcctggctgctccacgaaaccgctcttgttttgggcgataaggtattgctgcataagatagagcgcatcatccgtcgtatagccgatgctgcagatgaaggcttgcgtccggatggcagtatggtttacgagcattggaaggatggagataaatatgatctccagcgccagtggtgggtgcagtgcgagaacatcatcggccatatcgacctctatcagtatttccgtaccgaagagaatcttctggttgccatctcctgctggaactatgtagccaagcatctgctggataacaagaacggtgagtggcactgggctatactggaagatggtacggtgaacaaggaggatgataaggcgggtttctggaaatgtccttaccacaactcccgtatgtgtctcgaactcatcgaacgtgactattaa

SEQ ID NO: 13 - DNA, 369 bp, Prevotella copri
ggtgctgatgtaggttgccagggggaggtaggtgttgcctgcgccatggcttcggctgcagcctgtcagcttttcggaggcagtccggctcaggtagaatatgctgccgaaatgggattggagcatcatctcggaatgacctgcgacccagtttgtggactggttcagattccttgtatcgagcgaaatgcctttgctgccgcccgagctttggatgctgacctctacgcttccttctcagatggtcatcataccgtatctttcgaccgggtagttgaagtgatgcgacagacgggacatgatctaccttcgctttacaaggaaacaagcgaaggcggtttggcaaagggatttccaagagacatttag

SEQ ID NO: 14 - DNA, 1227 bp, Prevotella copri
aaggcgcatctctctgtgttccgtaatttcggaacagcacaagtgaagtatcaggatactgaagtggagtttgtcggtgcccgtaaggagagctatcagcgcgattccagaaagcctattgtggaagatggaacgctggaggatgaccagaaccgccgtgacttcaccatcaatgcgatggctatctgcctgaacaaagaccgttttggagaactcgtagatccttttgatggtgtctacgatatggaagatggtatcatcgccactccgctcgacccggatatcactttttctgatgacccgctgcgtatgatgcgttgtgtgcgtttcgctacccaactcaatttccagattgaggaggagacctatgatgcgctctcacgtaatgccgagcgcctgaagattatcagtgctgagcgcatctgcgacgagatgaacaagattatgctctccaagcacccaagcagcggtttctattacctgaaagatacaggtctgctcgatctcattctgccagaactggtggcgatggacaaggtagaaacgcgaaacggcagggctcataagaataattacgaccatacgatggaggtgctcgagaatgtctgcaagcattctgataacctctggctccgctgggctgctctcttccacgacatcggtaaaccgaagagcaagcgctgggacaacaatatcggctggacattccacagtcataatataataggtgcaaagatgattccgggtatcttccgccgtatgaaacttccgatggatgcgaagatgaagtatgtgcagaagctggtagaactccacatgcgtccgattgtgattgctgatgaggaggttaccgacagtgccgtacgccgcctgctgaatgatgccggcgatgacatcaacgacctgatgacgctctgcgaggctgatatcaccagcaagaaccaggtacgcaagcagcgttttctggataatttcaagatggtgcgcgagaaactggtcgatctgcaggaacgtgattataagcgacttcttcagccatgcatcgatggtaatgagattatggagatgttccatcttaccccttgccgtgaggtaggcaccctgaagcagtatctcaaggatgctgtattggataataaggtggctaatgagcgtgaaccgttgatggaacttctgatgaagaaggctcaggagatgggattggtaaatgcagaaaactcaaaatag

SEQ ID NO: 15 - DNA, 1563 bp, Prevotella copri
atggcttcaaagagatatcctttgggaatacaaacgttctccgaaatcgtgaaggggaattacttctatgctgacaagacggctatcgtctatcagttggctcattatgccaagtttcattttctgagtcgcccacgccgattcggaaaatccttgtttgtatctactctcaaggcctactttgaaggtaagaaagaactgttcaaggaccttgccatcgaacagatggagaaggaatggacggcatatcctgtcatccacttggatctgagctgcggtaaatattatagtttggagaatacatattcaattctaaacggaatactagaagtagaagagaaaaaatatggtttaaaggttaatccgatagatgagaagtcttttggctcacgtctcaagaatatattgcttgcagcagctgctcaaaccggtaaacaagctgttgttctcatcgacgagtatgatgctccaatgcatgattctgtaagtgacgaagagttgcagaagaccatccgcaacatcatgagagatttcttcagccccttgaagcagcaagagggaaacattcgctttgtattcatcacaggcatttccaagtttagtcagctcagcatctttagtgaattgaacaatctcaagattctcacgttgaaggatgaatacagtagctgttgtggtataaccaagagtgaattgactcagtatttccgtgagggtatcgaggagatggctgaacataatgggcttacttatgaggagaccttagagcaactcaagcagcattatgatggctaccacttcagtatcaatagcgaggatatcttcaatccttacagcattatcaatgctttggacgataaggagtttaatagctattggtttacatctggtacgcctacgttcctgatagaactgatgcagcagaagaatttggatatgatggacttaaatgatatctgggctagagctaaacgcttcgatgttcctactgagacgattactgaccctgtgccagtccttttccaaagtgggtatcttacgattaagggatatgacaagcagttaggtatgtattaccttagttttcctaatcaagaagttagacaaggtttttcggaaagcctttgcctatattataccccttcagaggtaggcgaacttgatgctatcgtatatgcttataaaaagaatgtgctcatcaatgacgacatgggagcctttatgcctcatttgaaggcattctatgataagttcccatatacgattatcaacaataatgagcgtcactatcaagccgtgatattcaccatcttcaccatgcttggcgaagatgtgaaggtggagcataccacttcggatggaagaatagaccttgtgctcaagacggataagagtatctttatctttgagttgaaatataagaagtctgccgacatcgccatggcgcaaatcagcgacaaggactatgccaaggcttttgccgatgatgggcgaaaggttgtgaaagtgggtattaacttctcggaaaaccagcgaagtatagaggattgggtgatagaataa

SEQ ID NO: 16 - DNA, 372 bp, Prevotella copri
atgattttcagtaacgataaagcaatatacgtacagatagccgagcgattgagcgatgagattctggcgggcaaatataaggaagacgaacgaataccgagcgtcagggaatatgccgtgctgctggaggtaaatgccaacacagccgtgaaggcatacgacctgcttgccaccgaagagatcatctacaacaagcgaggcttgggctactttgtttccgctggagccaagaagcagattaagaaaacccgcaaaaaggagtttatgaaagaaagacttccggaactcgcccgacaaatgcagcttctcgacatctccatcgatgaagtgaaggaagagttggagaagaatctcaaaaagataaagatatga

SEQ ID NO: 17 - DNA, 792 bp, Prevotella copri
atgtcggcttacgactttctgcgagctgtgaaagatgagattccgggaggttacaacttctgggtttatacgccggtagattatttctattcgcaagaacagactcctgtcatcatctttctgcatggagccagtctttgcggcaagaatctgaataaggtgagaagatacggaccgcttgatgccatcgtcaaggggcgcgatatcgatgcgctgaccatcgttccgcagaatccgggaggagcctggaatccgaagaaaatcatgggtatgctagactgggtgaaaaagaattacccatgcgattctaatagggtttatgtcttgggcatgagtttgggcggttatggcaccatggatgtctgcgccacttatcccgacaggatagctgccggcatggcgctgtgtggtggctgttcttataaggatgtgagtggattgggcgatttgcctttctggattatccatggcacagcagatagggcagtaccggtcaagcaatcgaaggttgtggtagataaacttgaaaaggatggcaaggatacccgattgatatacgactggtggaagggtgctaatcacggtacccctgcccgcgtgttttatctgaagaaaacctatcagtggctcttctctcacagcctttcagataaggacagacccgtgaaccgcaacatcagtattacgatgagcgatctgggcagagcatatggtgatgtgaacagaaatgcccctcagcctgaactcatcgatggtccgagtgtgatcaagcaagagggaaatgaatattaa

SEQ ID NO: 18 - DNA, 351 bp, Prevotella copri
atgaactttactttgttagtggtcgtcttattgacggcaatcgctttcgtgggtatcgtcatcgcgctctccaatgcgatagcaccccgttcgtacaatgcacagaagatggagccgtacgagtgtggtatccctacccgcggtaaatcgtggatgcaattccgggtaggttactatctgtttgccatcttgttcctgatgttcgaagtggaaacggtatttctgttcccttgggcagtcatcacccgcgaacttggcgtggcaggacttttcagtgttttatttttcctgattatattgattctgggccttgcttatgcctggaggaaaggagctttggaatggaagtaa

SEQ ID NO: 19 - DNA, 504 bp, Prevotella copri
atgaaagaaaagaactcttatatcggcggactcatacacggcgtcacttctctgctgacaggtatgaagacaaccatgaccgtattctgccgacgaaagactaccgaacaatatccggaaaaccgctcgacattgaaactctccgaccgtttccgtggcacgctgaccatgccccataatgataaaaatgaacatcggtgtgtagcttgcggtttatgccagatggcttgtcccaacgacactatcaaggtgaccggcgagatggttgaaacggaagacggcaagaaaaagaagatattggttaagtacgaatacgacctcggttcgtgcatgttctgccaactctgtgtgaatgcctgccctcacgatgccatcacgttcaaccaggattttgagcatgcagtgttcgaccgcacgaaactggtcatgacgctgaaccgtccgggcagccatgtagaagaaaaaaagaaacccgctgctcccaaagaagagacaacaaagtaa

SEQ ID NO: 20 - DNA, 20 bp, Artificial Sequence
  Synthetic oligonucleotide
ccggactcctgcccctgcaa

SEQ ID NO: 21 - DNA, 20 bp, Artificial Sequence
  Synthetic oligonucleotide
gttgcgccaggcactgcgat

SEQ ID NO: 22 - DNA, 20 bp, Artificial Sequence
  Synthetic oligonucleotide
cacrgtaaacgatggatgcc

SEQ ID NO: 23 - DNA, 16 bp, Artificial Sequence
  Synthetic oligonucleotide
ggtcgggttgcagacc

SEQ ID NO: 24 - DNA, 21 bp, Artificial Sequence
  Synthetic oligonucleotide
cacaacagccatagcgttcca

SEQ ID NO: 25 - DNA, 23 bp, Artificial Sequence
  Synthetic oligonucleotide
atcgcaaaaataagatgggcaaa

SEQ ID NO: 26 - DNA, 21 bp, Artificial Sequence
  Synthetic oligonucleotide
actcctacgggaggcagcagt

SEQ ID NO: 27 - DNA, 18 bp, Artificial Sequence
  Synthetic oligonucleotide
attaccgcggctgctggc

SEQ ID NO: 28 - DNA, 20 bp, Artificial Sequence
  Synthetic oligonucleotide
tacacggcgtcacttctctg

SEQ ID NO: 29 - DNA, 20 bp, Artificial Sequence
  Synthetic oligonucleotide
gatggttgaaacggaagacg

SEQ ID NO: 30 - DNA, 20 bp, Artificial Sequence
  Synthetic oligonucleotide
gctttcgtgggtatcgtcat

SEQ ID NO: 31 - DNA, 20 bp, Artificial Sequence
  Synthetic oligonucleotide
tgtttgccatcttgttcctg

SEQ ID NO: 32 - DNA, 20 bp, Artificial Sequence
  Synthetic oligonucleotide
ccatcctgaccgaaagaaaa

SEQ ID NO: 33 - DNA, 20 bp, Artificial Sequence
  Synthetic oligonucleotide
aaagcaggtggatgtatggg

SEQ ID NO: 34 - DNA, 20 bp, Artificial Sequence
  Synthetic oligonucleotide
cagagggcgtgaaatcgtat

SEQ ID NO: 35 - DNA, 20 bp, Artificial Sequence
  Synthetic oligonucleotide
atctgggcttcaacatcagg

SEQ ID NO: 36 - DNA, 260 bp, Prevotella copri
cgcggtaatacggaaggtccgggcgttatccggatttattgggtttaaagggagcgtaggccggagattaagcgtgttgtgaaatgtagacgctcaacgtctgcactgcagcgcgaactggtttccttgagtacgcacaaagtgggcggaattcgtggtgtagcggtgaaatgcttagatatcacgaagaactccgattgcgaaggcagctcactggagcgcaactgacgctgaagctcgaaagtgcgggtatcgaacag

SEQ ID NO: 37 - DNA, 260 bp, Prevotella copri; identical to SEQ ID NO: 36

SEQ ID NO: 38 - DNA, 260 bp, Prevotella copri
cgcggtaatacggaaggtccgggcgttatccggatttattgggtttaaagggagcgtaggccggagattaagcgtgttgtgaaatgtagatgctcaacatctgcactgcagcgcgaactggtttccttgagtacgcacaaagtgggcggaattcgtggtgtagcggtgaaatgcttagatatcacgaagaactccgattgcgaaggcagctcactggagcgcaactgacgctgaagctcgaaagtgcgggtatcgaacag

SEQ ID NO: 39 - DNA, 260 bp, Prevotella copri; identical to SEQ ID NO: 36

SEQ ID NO: 40 - DNA, 260 bp, Prevotella copri; identical to SEQ ID NO: 36

SEQ ID NO: 41 - DNA, 260 bp, Prevotella copri; identical to SEQ ID NO: 36

(19) United States
(12) Patent Application Publication
(10) Pub. No.: US 2016/0186261 A1
(43) Pub. Date: Jun. 30, 2016

(54) PREVOTELLA COPRI AND ENHANCED SUSCEPTIBILITY TO ARTHRITIS

(71) Applicants: Jose U. Scher, Jersey City, NJ (US); Andrew Sczesnak, Berkeley, CA (US); Randy S. Longman, New York, NY (US); Nicola Segata, Trento (IT); Carles Ubeda, Valencia (ES); Eric G. Pamer, Guilford, CT (US); Steven B. Abramson, Rye, NY (US); Curtis Huttenhower, Needham, MA (US); Dan R. Littman, New York, NY (US); Hannah Fehlner-Peach, New York, NY (US)

(72) Inventors: Jose U. Scher, Jersey City, NJ (US); Andrew Sczesnak, Berkeley, CA (US); Randy S. Longman, New York, NY (US); Nicola Segata, Trento (IT); Carles Ubeda, Valencia (ES); Eric G. Pamer, Guilford, CT (US); Steven B. Abramson, Rye, NY (US); Curtis Huttenhower, Needham, MA (US); Dan R. Littman, New York, NY (US); Hannah Fehlner-Peach, New York, NY (US)

(21) Appl. No.: 14/532,586

(22) Filed: Nov. 4, 2014

Related U.S. Application Data
(60) Provisional application No. 61/899,454, filed on Nov. 4, 2013.

(51) Int. Cl. C12Q 1/68; G01N 33/569

(52) U.S. Cl. CPC C12Q 1/6883; G01N 33/56911; C12Q 1/689; C12Q 2600/106; C12Q 2600/136; G01N 2800/50; G01N 2800/102; G01N 2800/52; G01N 2500/10

(57) ABSTRACT

Methods, reagents and compositions thereof for predicting risk for NORA onset in susceptible individuals, diagnosing NORA onset, and/or evaluating efficacy of a therapeutic regimen for treating RA are described herein. Determining the amount of at least one of SEQ ID NOs: 1-19 and/or at least one of a KO presented in either of Tables S4 or S5 serves as a biomarker for the above indications.

### CROSS REFERENCE TO RELATED APPLICATIONS

This application claims priority under 35 USC §119(e) from U.S. Provisional Application Ser. No. 61/899,454, filed Nov. 4, 2013, which application is herein specifically incorporated by reference in its entirety.

### GOVERNMENTAL SUPPORT

The research leading to the present invention was supported, at least in part, by GO grant 1RC2AR058986, K23 grant K23AR064318, and RO1 grant R01AI042135 awarded by the National Institutes of Health, and Grant No. 1144247 awarded by the National Science Foundation. Accordingly, the Government has certain rights in the invention.

### FIELD OF THE INVENTION

Diagnostic and prognostic methods pertaining to inflammatory and autoimmune disorders are described herein. More particularly, diagnostic and prognostic methods relating to Rheumatoid Arthritis (RA) are set forth herein.

### BACKGROUND OF THE INVENTION

Rheumatoid Arthritis (RA) is a chronic, systemic inflammatory disorder of unknown etiology that predominantly affects synovial joints. RA is, moreover, an autoimmune disease that affects about 1% of the Caucasian population, with a higher ratio of females afflicted (Lee et al. 2001; Lancet 358:903-911). The disease can occur at any age, but it is most common in human subjects between 30 to 55 years old (Sweeney et al. 2004; Int. J. Biochem. Cell Biol. 36:372-378). The incidence of RA increases with age.

Although the cause of RA is unknown, certain genetic and infectious factors have been implicated in RA pathogenesis (Smith et al. 2002; Ann. Intern. Med. 136:908-922). Soluble cytokines and chemokines, such as IL-1β, TNFα, IL-1ra, IL-6, IL-8, MCP-1 and serum amyloid A (SAA), have been shown to be associated with rheumatoid arthritis (Szekanecz et al. 2001; Curr. Rheumatol. Rep. 3:53-63; Gabay et al. 1997; J. Rheumatol. 24:303-308; Arvidson et al. 1994; Ann. Rheum. Dis. 53:521-524; De Benedetti et al. 1999; J. Rheumatol. 26:425-431.

The predominant symptoms of RA are pain, stiffness, and swelling of peripheral joints. Of the synovial joints, RA most commonly affects the joints of the hands, feet and knees (Smolen et al. 1995; Arthritis Rheum. 38:38-43). RA can also, however, affect the spine with devastating results and atlanto-axial joint involvement is common in more progressed disease. Extra-articular involvement is a hallmark of RA, which can range from rheumatoid nodules to life-threatening vasculitis (Smolen et al. 2003; Nat. Rev. Drug Discov. 2:473-488). The disease manifests with variable outcome, ranging from mild, self-limiting arthritis to rapidly progressive multi-system inflammation, which is associated with pronounced morbidity and mortality (Lee et al. 2001; ibid; Sweeney et al 2004; ibid). Joint damage occurs early in the course of the disease as evidenced by the fact that bony erosions are detected in 30 percent of patients at the time of diagnosis (van der Heijde 1985; Br. J. Rheumatol. 34 (Suppl 2): 74-78).

Seven diagnostic criteria recognized by The American Rheumatism Association (ARA) (Arnett et al. 1988; Arthritis Rheum. 31:315-324) are used to diagnose RA. The ARA criteria include: 1) morning stiffness in and around joints lasting at least 1 hour before maximal improvement; 2) soft tissue swelling (arthritis) of 3 or more joint areas observed by a physician; 3) swelling (arthritis) of the hand joints; 4) symmetric swelling (arthritis); 5) rheumatoid nodules; 6) elevated levels of serum rheumatoid factor (RF); and 7) radiographic changes in hand and/or wrist joints. For a definitive diagnosis of RA, the first four criteria must be present for a minimum of six weeks. The RA test measures rheumatoid factor—the IgM autoantibody reactive with Fc region epitopes of the IgG molecule (Corper et al. 1997; Nat. Struct. Biol. 4: 374-381). Although RF is primarily associated with RA, these antibodies can be detected in sera from normal elderly people, healthy individuals, and patients with other autoimmune disorders or chronic infections (Williams 1998) and thus, have low disease specificity.

RA is typically treated with a variety of drugs that can be categorized as follows: nonsteroidal anti-inflammatory drugs (NSAIDs); disease-modifying anti-rheumatic drugs (DMARDs), steroids, and analgesics. NSAID drugs (such as ibuprofen and aspirin) reduce swelling and pain associated with the disease but offer only symptomatic relief. DMARDs include sulfasalazine and methotrexate, as well as biological agents, such as Infliximab, Etanercept, Adalimumab and Anakinra. All of the above therapeutics, however, fail to address the underlying cause of RA.

In view of the above, new methods for use in the accurate diagnosis, prognosis, and/or monitoring of patients with rheumatoid arthritis are urgently needed. Methods described herein address these needs.

The citation of references herein shall not be construed as an admission that such is prior art to the present invention.

### SUMMARY OF THE INVENTION

Rheumatoid arthritis (RA), one of the most prevalent systemic autoimmune diseases, has been proposed to be caused by a combination of genetic and environmental factors. Animal models have suggested a role for intestinal bacteria in supporting the systemic immune response required for joint inflammation. As described herein, the present inventors performed 16S and shotgun sequencing on stool samples from 114 rheumatoid arthritis patients and controls and identified the presence of Prevotella copri (P. copri) as strongly correlated with disease in new-onset untreated rheumatoid arthritis (NORA) patients. Increases in Prevotella abundance correlated with a reduction in Bacteroides and a loss of reportedly beneficial microbes in NORA subjects. The present inventors also identified unique Prevotella genes that correlated with disease. Colonization of mice, moreover, revealed the ability of P. copri to dominate the intestinal microbiota and resulted in an increased sensitivity to colitis and inflammatory arthritis. Results presented herein, therefore, identify P. copri as having a role in the pathogenesis of RA. See also Scher et al. (2013, eLife 2:e01202), the entire content of which is incorporated herein by reference.

More particularly, the present inventors used high-throughput 16S and shotgun sequencing of fecal samples to reveal an association of untreated rheumatoid arthritis with P. copri, a human gut microbe sufficient to exacerbate intestinal and joint inflammation in mouse models. In so doing, the present inventors have identified 17 P. copri genes (open reading frames) that are correlated with disease and 2 P. copri bacterial genes that are inversely correlated with disease.

Based on these findings, the presence and/or abundance of any one of the 17 P. copri genes (open reading frames) that are correlated with disease and/or any one of the 2 P. copri bacterial genes that are inversely correlated with disease (or positively correlated with a healthy state) in a human subject, particularly in the intestinal tract, can be used as a diagnostic indicator for RA onset, as a predictive indicator for RA onset in susceptible individuals, and as a prognostic indicator for RA patients receiving treatment therefor.

Further to the above, any one of SEQ ID NOs: 1-19 and variants thereof can each be used alone or in combination in methods described herein for diagnostic, prognostic and/or therapeutic applications, as well as compositions and screening assays.

In accordance with the findings found herein, a method for determining whether a subject has new onset rheumatoid arthritis (NORA) or is at risk for developing NORA is presented, the method comprising isolating a biological sample from the subject and determining the amount of at least one NORA indicator open reading frame in a biological sample obtained from the subject, wherein the at least one NORA indicator open reading frame is identified in Table S3.

In a particular embodiment thereof, a method for determining whether a subject is at risk for developing new onset rheumatoid arthritis (NORA) is presented, the method comprising: isolating a biological sample from the subject; processing the biological sample to generate a cellular lysate comprising nucleic acid sequences; analyzing the nucleic acid sequences to measure an amount of at least one NORA marker open reading frame in the cellular lysate, wherein the at least one NORA marker open reading frame is identified in Table S4 and wherein detecting the presence or absence of at least one NORA marker open reading frame in the cellular lysate is correlated with increased risk for developing NORA in the subject.

In a particular embodiment thereof, the cellular lysate generated has reduced protein content relative to unprocessed cellular lysate. In a further embodiment thereof, the cellular lysate is essentially free of cellular proteins (wherein cell protein concentration is reduced by, e.g., at least 90%, 95%, 96%, 97%, 98%, or 99%, or 100% relative to unprocessed cellular lysate).

In another particular embodiment, the at least one NORA marker open reading frame is at least 2, 3, 4, 5, 6, 7, 8, 9, 10, 11, 12, 13, 14, 15, 16, 17, 18, or 19 NORA marker open reading frames.

In another particular embodiment, the ratio of P. copri to other microorganisms in the biological sample has increased and, as a consequence thereof, P. copri may represent 5-70% of the total microbiome. This relative increase in P. copri is accompanied by a reduction in other taxa, particularly Bacteroides.

In an embodiment of the method, at least one NORA indicator open reading frame is a NORA-specific open reading frame and the presence or increased amount of the at least one NORA-specific open reading frame indicates that the subject has NORA or is at risk for developing NORA, wherein the increased amount is determined relative to an amount detected in a healthy control and at least one NORA-specific open reading frame present or increased in amount is gene_id_62568 (SEQ ID NO: 1); gene_id_29546 (SEQ ID NO: 2); gene_id_90049 (SEQ ID NO: 3); gene_id_62569 (SEQ ID NO: 4); gene_id_55079 (SEQ ID NO: 5); gene_id_83051 (SEQ ID NO: 6); gene_id_79069 (SEQ ID NO: 7); gene_id_68986 (SEQ ID NO: 8); gene_id_54057 (SEQ ID NO: 9); gene_id_45456 (SEQ ID NO: 10); gene_id_29407 (SEQ ID NO: 11); gene_id 45366 (SEQ ID NO: 12); gene_id_81143 (SEQ ID NO: 13); gene_id_45134 (SEQ ID NO: 14); gene_id_17194 (SEQ ID NO: 15); gene_id 68779 (SEQ ID NO: 16); or gene_id_59356 (SEQ ID NO: 17). See FIG. 8.

In another embodiment of the method, at least one NORA indicator open reading frame is a healthy-specific open reading frame and the absence or decreased amount of the at least one healthy-specific open reading frame indicates that the subject has new onset rheumatoid arthritis (RA) or is at risk for developing RA, wherein the decreased amount is determined relative to an amount detected in a healthy control and the at least one healthy-specific open reading frame absent or decreased in amount is gene_id_3694 (SEQ ID NO: 18) or gene_id_3690 (SEQ ID NO: 19). In yet another embodiment of the method, the at least one NORA marker open reading frame is a healthy-specific open reading frame and the presence of at least one healthy-specific open reading frame indicates that the subject is at reduced risk for developing NORA, wherein the at least one healthy-specific open reading frame is gene_id_3694 (SEQ ID NO: 18) or gene_id_3690 (SEQ ID NO: 19). See FIG. 8.

In a particular embodiment thereof, the subject is selected for evaluation because the subject has a familial history of RA and/or exhibits at least one of the seven diagnostic criteria recognized by the ARA to diagnose RA. The ARA criteria include: 1) morning stiffness in and around joints lasting at least 1 hour before maximal improvement; 2) soft tissue swelling (arthritis) of 3 or more joint areas observed by a physician; 3) swelling (arthritis) of the hand joints; 4) symmetric swelling (arthritis); 5) rheumatoid nodules; 6) elevated levels of serum rheumatoid factor (RF); and 7) radiographic changes in hand and/or wrist joints.

In a particular embodiment of the method, the biological sample is fecal material, biopsies of specific organ tissues, including large and small intestinal biopsies, synovial fluid, and synovial fluid biopsies. In an embodiment wherein the biological sample is fecal material, the method may further comprise processing the fecal material to generate a fecal bacterial sample. Such methods are described herein and are known in the art. See, for example, Hamilton et al. (Am J Gastroenterol. 107(5):761-7, 2012), the entire content of which is incorporated herein by reference. Such protocols generate processed fecal material (fecal filtrate), which has reduced volume and fecal aroma and from which cellular lysates may be generated. Methods for generating a cellular lysate (e.g., a cellular lysate having reduced protein content relative to unprocessed cellular lysate) directly from fecal material are also described herein in the Examples and known in the art.

The method may further comprise assessment of familial history of RA in the subject, clinical symptoms of RA, ACPA/RF levels, or Th17/Treg levels in the subject.

The method may further comprise treating a subject identified as at risk for developing NORA or as having NORA with an agent or a combination of agents used to treat RA. Such agents include, without limitation, antibiotics (e.g., vancomycin); nonsteroidal anti-inflammatory drugs (NSAIDs); disease-modifying anti-rheumatic drugs (DMARDs), steroids (e.g., prednisone), and analgesics. NSAID drugs (such as ibuprofen and aspirin) reduce swelling and pain associated with the disease but offer only symptomatic relief. DMARDs include sulfasalazine and methotrexate, as well as biological agents, such as Infliximab, Etanercept, Adalimumab and Anakinra. A skilled practitioner would be aware of suitable dosing regimens for treating a patient in need thereof.

In a further embodiment of the method, the amount of at least one NORA indicator open reading frame in the biological sample is determined by nucleic acid sequencing. In a more particular embodiment, the nucleic acid sequencing is shotgun sequencing. As described herein and understood in the art, such sequencing may be performed using sequencers available from 454 Life Sciences or Illumina, Inc.

In a particular embodiment of the method, the nucleic acid sequencing detects open reading frames comprising at least one of SEQ ID NOs: 1-19 and the amount of the open reading frames comprising at least one of SEQ ID NOs: 1-19 is compared to an amount detected for each of the respective SEQ ID NOs: in a biological sample obtained from a healthy subject to determine a fold increase or decrease in the at least one of SEQ ID NOs: 1-19 in the biological sample.

In another embodiment of the method, the amount of the at least one NORA indicator open reading frame is determined using a reagent that specifically binds to the at least one NORA indicator open reading frame. Reagents useful for such applications include, without limitation, an antibody, an antibody derivative, an antibody fragment, a nucleic acid probe, an oligonucleotide, and an oligonucleotide primer pair specific for any one of SEQ ID NOs: 1-19. In a particular embodiment, the reagent is an oligonucleotide primer pair corresponding to primers that anneal in a sequence specific manner to any one of SEQ ID NOs: 1-19 and which anneal to the sequence identifier at a distance suitable for generating a product following a polymerase chain reaction amplification. Exemplary primers for gene_id 3690 include: Forward primer: TACACGGCGTCACTTCTCTG (SEQ ID NO: 28) and Reverse primer: GATGGTTGAAACGGAAGACG (SEQ ID NO: 29); for gene_id_3694: Forward primer: GCTTTCGTGGGTATCGTCAT (SEQ ID NO: 30) and Reverse primer: TGTTTGCCATCTTGTTCCTG (SEQ ID NO: 31); for gene_id_62568: Forward primer: CCATCCTGACCGAAAGAAAA (SEQ ID NO: 32) and Reverse primer: AAAGCAGGTGGATGTATGGG (SEQ ID NO: 33); and for gene_id_62569: Forward primer: CAGAGGGCGTGAAATCGTAT (SEQ ID NO: 34) and Reverse primer: ATCTGGGCTTCAACATCAGG (SEQ ID NO: 35).

P. copri genome specific primers such as, for example, Forward primer: CCGGACTCCTGCCCCTGCAA (SEQ ID NO: 20) and Reverse primer: GTTGCGCCAGGCACTGCGAT (SEQ ID NO: 21); and Prevotella 16S primers: Forward primer: CACRGTAAACGATGGATGCC (SEQ ID NO: 22) and Reverse primer: GGTCGGGTTGCAGACC (SEQ ID NO: 23) may be used for amplification of P. copri to detect the presence of same in a sample.

In yet another embodiment of the method, determining the amount of the at least one NORA indicator open reading frame includes at least one assay selected from the group consisting of nucleic acid sequencing, PCR amplification, a competitive binding assay, a non-competitive binding assay, a radioimmunoassay, immunohistochemistry, an enzyme-linked immunosorbent assay (ELISA), a sandwich assay, a gel diffusion immunodiffusion assay, an agglutination assay, dot blotting, a fluorescent immunoassay such as fluorescence-activated cell sorting (FACS), a chemiluminescence immunoassay, an immunoPCT immunoassay, a protein A or protein G immunoassay, and an immunoelectrophoresis assay.

Also encompassed herein is a method for evaluating therapeutic efficacy of an agent administered to a patient with RA, the method comprising: isolating a biological sample from the patient with RA before and after administering the agent; processing each of the biological samples to generate a cellular lysate comprising nucleic acid sequences of each of the biological samples; analyzing the nucleic acid sequences of each of the biological samples to measure an amount of at least one of SEQ ID NOs: 1-19 before administration of the agent and an amount of least one of SEQ ID NOs: 1-19 after administration of the agent; and comparing the amount of the least one of SEQ ID NOs: 1-19 determined before and after administration of the agent, wherein a decrease in the amount of at least one of SEQ ID NOs: 1-17 and/or an increase in the amount of at least one of SEQ ID NO: 18 or SEQ ID NO: 19 after administration of the agent is a positive indicator of the therapeutic efficacy of the agent for RA.

Also encompassed herein is a method for identifying a test substance that modulates levels of Prevotella copri in a subject, said method comprising a) isolating a biological sample from the subject and determining the amount of the at least one of SEQ ID NOs: 1-19 in the biological sample obtained from said subject; b) contacting the biological sample with a test substance; and c) determining the amount of the at least one of SEQ ID NOs: 1-19 in the biological sample after contact with the test substance, wherein an alteration in the amount of the at least one of SEQ ID NOs: 1-19 determined in step c) relative to the amount determined in step a) identifies the test substance as a modulator of Prevotella copri levels. In a particular embodiment, a decrease in the amount of the at least one of SEQ ID NOs: 1-17 determined in step c) when compared to the amount of the at least one of SEQ ID NOs: 1-17, respectively, determined in step a) indicates that the test substance is a potential agent for treating or preventing RA in a subject. In another embodiment, an increase in the amount of the at least one of SEQ ID NOs: 18 or 19 determined in step c) when compared to the amount of the at least one of SEQ ID NOs: 18 or 19, respectively, determined in step a) indicates that the test substance is a potential agent for treating or preventing RA in a subject.

Also encompassed herein is a composition for the prediction or diagnosis of NORA or the prognosis of a NORA patient undergoing a therapeutic regimen, the composition comprising specific detection reagents for determining the amount of at least one of SEQ ID NOs: 1-19 and a buffer compatible with the activity of the specific detection reagents. In a particular embodiment, the specific detection reagents comprise a nucleic acid probe, an oligonucleotide, or an oligonucleotide primer pair specific for at least one of SEQ ID NOs: 1-19. In a still further embodiment, the specific detection reagents comprise at least one sequence-specific oligonucleotide that binds specifically to any one of SEQ ID NOs: 1-19. The specific detection reagents may be labeled with a detectable moiety or moieties. In a particular embodiment, a specific detection reagent is linked to a moiety that confers immobilization properties, and/or immobilized on a solid phase support.

By “solid phase support or carrier” is intended any support capable of binding an oligonucleotide, antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present methods and/or compositions. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art are aware of many other suitable carriers for binding oligonucleotide, antibody, or antigen, and are able to ascertain the same by use of routine experimentation.

Other objects and advantages will become apparent to those skilled in the art from a review of the following description which proceeds with reference to the following illustrative drawings.

### BRIEF DESCRIPTION OF THE DRAWINGS

FIG. 1. Differences in the relative abundance of Prevotella and Bacteroides in 114 subjects with and without arthritis, determined by 16S sequencing (regions V1-V2, 454 platform). (a) LEfSe (Segata et al., 2011) was used to compare the abundances of all detected clades among all groups, producing an effect size for each comparison (see Methods). All results shown are highly significant (q<0.01) by Kruskal-Wallis test adjusted with the Benjamini-Hochberg procedure for multiple testing, except that indicated with an asterisk, which is significant at q<0.05. Negative values (left) correspond to effect sizes representative of NORA groups, while positive values (right) correspond to effect sizes in HLT subjects. Prevotella was found to be over-represented in NORA patients, while Bacteroides was over-represented in all other groups. (b) The Bray-Curtis distance between all subjects was calculated and used to generate a principal coordinates plot in MOTHUR (Schloss et al., 2009). The first two components are shown. Subjects with an abundance of Prevotella greater than 10% were colored red. Other subjects were colored according to their Bacteroides abundance as shown. NORA subjects (stars) primarily cluster together according to their Prevotella abundance, and the x-axis is representative of differences in the relative abundance of Prevotella and Bacteroides. (c) The abundances of Prevotella (red) and Bacteroides (blue) are shown for all subjects, sorted in order of decreasing Prevotella abundance (>5%) and increasing Bacteroides abundance.

FIG. 2. Homology-based classification of patient-associated Prevotella. Four NORA subjects with a high abundance of Prevotella OTU4 were selected for shotgun sequencing and metagenome assembly. (a) The resulting metagenomic contigs were used to generate a phylogenomic tree with PhyloPhlAn (Segata et al., 2013). (b) Assemblies were filtered by alignment to the reference P. copri DSM 18205 genome, keeping contigs with at least one 300 bp region aligned at 97% identity or greater. The resulting draft patient-derived P. copri assemblies were aligned to one another, the reference P. copri genome, and two distinct Prevotella taxa (P. buccae and P. buccalis). Colored arcs represent assemblies as labeled, lines connecting arcs represent regions of >97% identity >1 kb in length, and gray lines dividing colored arcs represent boundaries between contigs. These results demonstrate that Prevotella OTU4, OTU12, and OTU934 form a clade with P. copri (left, red highlighted subtree) that is genetically distinct from more distant Prevotella taxa.

FIG. 3. Comparison of P. copri genomes from healthy and NORA subjects. (a) Comparative coverage of the draft Prevotella copri DSM 18205 genome between individuals and within healthy and NORA groups. Gray points are median fragments per kilobase per million (FPKM) for 1-kb windows, gray lines within the plot are the interquartile range for each window, red and blue lines the LOWESS-smoothed average for NORA and healthy groups, respectively. Gray lines on the horizontal axis represent boundaries between assembled contigs. Regions are variably covered between subjects and groups, with several genomic islands lacking overall or especially variable (dark blue lines below the plot). (b) The presence (blue) or absence (gray) of previously-reported P. copri-unique marker genes (Segata et al., 2012) in 11 stool samples from 5 subjects of the Human Microbiome Project (HMP) are shown as a heatmap. The present inventors report, in columns, only those P. copri-specific markers showing variable presence/absence patterns across the considered HMP samples. Each row represents a different sample collection date, groups of rows represent subjects, and groups of columns correspond to different variably covered genomic islands. Strains of P. copri are defined by the presence and absence of particular genes, which remain stable for at least 6 months in these individuals. All inter- and intra-individual comparisons between rows are highly statistically significant (p<<0.001, see Methods). (c) The P. copri pangenome was identified by finding P. copri ORFs in all HMP and NORA cohort subjects, and the presence or absence of these ORFs was calculated for each subject (see Methods, FIG. S4). Several ORFs are statistically significant biomarkers between healthy and NORA status (q<0.25) (Table S3, see Methods).

FIG. 4. Metabolic pathway representation in the microbiome of healthy and NORA subjects. HUMAnN (Abubucker et al., 2012) was applied to metagenomic reads (paired-end, 100 nt, Illumina platform) from NORA subjects (n=14) and healthy controls (n=5) to quantitate the abundances of hierarchically related KEGG modules in these samples (see Methods and Table S2). LEfSe (Segata et al., 2011) was used to find statistically significant differences between groups at an alpha cutoff of 0.001 and an effect size cutoff of 2.0. Results shown here are highly significant (p<0.001) and represent large differences between groups. Modules highlighted in red are over-abundant in NORA samples while modules highlighted in blue are over-abundant in healthy samples. Prevotella-dominated NORA metagenomes have a dearth of genes encoding vitamin and purine metabolizing enzymes, and an excess of cysteine metabolizing enzymes.

FIG. 5. Relationship of host HLA genotype to abundance of Prevotella copri (OTU4, OTU12, and OTU934 combined relative abundance). The HLA-class II genotype of all subjects was determined by sequence-based typing methodology (see Methods). Groups were subdivided by the presence or absence of shared-epitope RA risk alleles (+/−SE as indicated above) and correlated with relative abundance of intestinal P. copri. A statistically significant correlation is seen between P. copri abundance and the genetic risk for rheumatoid arthritis in NORA (red stars) and healthy (blue circles) subjects by Welch's two-tailed t-test.

FIG. 6. Colonization with P. copri dominates the colonic microbiome and exacerbates local and systemic inflammatory responses. (a) DNA was extracted from fecal pellets of media-gavaged mice and P. copri-gavaged mice 2 weeks after colonization and assayed by QPCR with P. copri specific primers compared to universal 16S. (b) Relative abundance of bacterial families in fecal DNA from media-gavaged and P. copri-colonized mice (shown in duplicate) by high-throughput 16S sequencing (regions V1-V2, 454 platform). (c) C57BL/6 mice colonized with P. copri (n=15) or media alone (n=13) controls were exposed to DSS for seven days and percent of starting body weight is shown. Composite data from three representative experiments are shown. (d) Representative colonoscopic images of mice colonized with P. copri or media gavage following DSS-induced colitis. Endoscopic colitis score for five individual animals is displayed. (e, f) Gross pathology (e) and histology (f) of colons from mice colonized with P. copri or media gavage following DSS-induced colitis.

FIG. 7. Colonization with P. copri exacerbates local and systemic inflammatory responses. (a, b) P. copri-colonized (n=9) compared to media-gavaged (n=7) mice were immunized with type II collagen/CFA precipitating collagen induced arthritis (CIA). Arthritis was scored as a composite score of 4 foot pads each scored 0-4 (a) and ankle thickness was recorded at 5 weeks (b). Data from two of four representative experiments are shown.

FIG. 8A-S. Nucleic acid sequences corresponding to SEQ ID NOs: 1-19.

FIG. 9. Schematic of procedure for isolation of P. copri from rheumatoid arthritis (RA) patient feces.

FIG. 10. Nucleic acid sequence alignment of the V3-V5 region of the 16S rDNA gene from colonies isolated from RA patient feces aligns to the reference P. copri V3-V5 16S rDNA gene.

FIG. 11A-B. Graphs revealing that patient P. copri induces Th17 in colons of colonized mice. A. Lamina propria cells were isolated from colons of media-, patient P. copri-, P. copri reference-, or B. theta-gavaged mice 17 days after colonization. Data are representative of three independent experiments. B. Representative flow cytometry plots of data summarized in (A).

FIG. S1. (a,b) Gut microbiota richness and diversity are similar among RA groups and healthy controls. (c) Phyla abundance by group. No significant differences were found at this taxonomic level. (d) Family abundance by group. NORA subjects have a significant increase in Prevotellaceae (red) and a concomitant decrease in Bacteroidaceae (blue) by FDR-adjusted Kruskal-Wallis test (q<0.01).

FIG. S2. The representative 16S sequenced reads for Prevotella OTU4, OTU12, and OTU934 were aligned with MUSCLE (Edgar, 2004) and clustered with FastTree (Price et al., 2010). All three Prevotella OTUs cluster with the full-length reference 16S sequence of P. copri.

FIG. S3. Recovery of Prevotella copri pangenome from HMP/RA shotgun reads and determination of presence/absence of P. copri ORFs by alignment of reads to pangenome gene catalog. (a) Genes were called present in a sample if they were covered by aligned reads at an identity threshold of >97% over >97% of their length (red lines). (b) ORFs were called on contigs using MetaGeneMark (Zhu et al., 2010) and were dereplicated with UCLUST (Edgar, 2010) at an identity threshold of 97% (red line). (c) Recovery of a sample's P. copri pangenome saturated at approximately 7 million reads (red line). The present inventors therefore excluded samples with less than 7 million P. copri reads, defined as P. copri abundance determined by MetaPhlan (Segata et al., 2012) multiplied by the total number of quality-filtered reads. Samples with P. copri abundance likely misestimated (i.e., those with <3000 ORFs present) were also excluded (Table S2). (d) Contigs were said to have originated from P. copri if they had at least one hit >97% identity over >300 bp (red lines).

FIG. S4. Metagenomic context of discriminative biomarker ORFs. ORFs found in the P. copri DSM 18205 reference genome are colored red, while those identified as differentially present in healthy and NORA groups are indicated with red asterisks. (a) Two ORFs, 3690 and 3694, are healthy-specific, occur on the same contig, and encode different components of the same NADH:quinone oxidoreductase. (b) Similarly, ORFs 62568 and 62569 occur on the same contig, are NORA-specific, and encode components of the same iron ABC transporter.

FIG. S5. P. copri colonization exacerbates chemically induced colitis. (a) DNA was extracted from fecal pellets of media, P. copri, and B. thetaiotamicron gavaged mice 2 weeks after colonization and assayed by QPCR with P. copri or Bacteroides specific primers compared to universal 16S amplicon. (b) C57BL/6 mice colonized with P. copri (n=10) or B. theta (n=10) were exposed to DSS for seven days and percent of starting body weight is shown. (c) Percent of total CD4⁺ T-cells in the colonic lamina propria expressing IL-17 (Th17) or IFNγ (Th1) following PMA/ionomycin stimulation or expressing Foxp3 (Treg).

FIG. S6. P. copri predominates in the colon of gavaged mice. (a, b) Fecal DNA was isolated from luminal samples derived from the ileum and cecum of C57BL/6 mice 2 weeks following gavage as described in Methods. QPCR was normalized both by universal 16S amplification (a) and total ng of P. copri per mg of luminal sample (b).

### DETAILED DESCRIPTION

Rheumatoid arthritis is a highly prevalent systemic autoimmune disease with predilection for the joints. If left untreated, RA can lead to chronic joint deformity, disability, and increased mortality. Despite recent advances towards understanding its pathogenesis (McInnes and Schett, 2011), the etiology of RA remains elusive. It is currently believed to be a complex polygenic and multifactorial disorder. Many genetic susceptibility risk alleles have been discovered and validated (Stahl et al., 2010) but are insufficient to explain disease incidence. Environmental factors are therefore required for the onset of RA (McInnes and Schett, 2011).

Among environmental factors, the intestinal microbiota has emerged as a possible candidate responsible for the priming of aberrant systemic immunity in RA (Scher and Abramson, 2011). The microbiota encompasses hundreds of bacterial species whose products represent an enormous antigenic burden that must largely be compartmentalized to prevent immune system activation (Littman and Pamer, 2011). In the healthy state, intestinal lamina propria cells of both innate and adaptive immune systems cooperate to maintain a state of physiological homeostasis. In RA, there is increased production of both self-reactive antibodies and pro-inflammatory T lymphocytes that are thought to contribute to disease pathogenesis. Although mechanisms for targeting of synovium by inflammatory cells have not been elucidated, studies in animal models suggest that both T cell and antibody responses are involved in pathogenesis. Moreover, an imbalance in the composition of the gut microbiota (dysbiosis) can alter local T-cell responses and modulate systemic inflammation. The Th17 cell differentiation pathway, which has been studied extensively in mouse and human, is required for the onset of disease in multiple models of autoimmunity and has been implicated by genetic and therapeutic studies as having a central role in humans with inflammatory bowel disease, psoriasis, and several arthritides (Seiderer et al., 2008, Lowes et al., 2008, Hirota et al., 2007). Th17 cells are most prevalent in the intestinal lamina propria, where they differentiate in response to specific constituents of the commensal microbiota. Mice rendered deficient for the microbiota (germ-free) lack Th17 cells, and colonization with segmented filamentous bacteria (SFB), a commensal microbe commonly found in mammals, is sufficient to induce Th17 cell differentiation (Ivanov et al., 2009, Sczesnak et al., 2011).

In several animal models of arthritis, mice are persistently healthy when raised in germ-free conditions. However, the introduction of specific gut bacterial species is sufficient to induce joint inflammation (Wu et al., 2010, Abdollahi-Roodsaz et al., 2008, Rath et al., 1996), and antibiotic treatment both prevents and abrogates a rheumatoid arthritis-like phenotype in several mouse models. Upon mono-colonization of arthritis-prone K/BxN mice with SFB, the induced Th17 cells potentiate inflammatory disease (Wu et al., 2010). An imbalance in intestinal microbial ecology, in which SFB is dominant, may result in reduced proportions or functions of anti-inflammatory regulatory T cells (Treg) and in a predisposition towards autoimmunity. Dysbiosis appears to affect not only the local immune response, but also systemic inflammatory processes, and may explain, at least in part, reduced Treg cell function in RA patients (Zanin-Zhorov et al., 2010). Thus, T cells whose functions are dictated by intestinal commensal bacteria can be effectors of pathogenesis in tissue-specific autoimmune disease.

Although recent studies of the human microbiome (HMP, 2012, Arumugam et al., 2011) have characterized the composition and diversity of the healthy gut microbiome, and disease-associated studies revealed correlations between taxonomic abundance and some clinical phenotypes (Morgan et al., 2012, Frank et al., 2011, Qin et al., 2012), a role for distinct microbial enterotypes and metagenomic markers in systemic inflammatory disease has not been defined. RA has long been suggested to be associated with infections or with dysbiosis of the microbiota (Scher and Abramson, 2011). Although treatment with antibiotics has been a therapeutic modality in RA for decades, no microbial organism has been shown to be associated with the disease.

To explore the role of the fecal microbiota in arthritis in humans, the present inventors analyzed the fecal microbiota in patients with RA. The present inventors used 16S ribosomal RNA gene sequencing to classify the microbiota in patients with new-onset (untreated) RA, chronic (treated) RA, psoriatic arthritis, and age- and ethnicity-matched healthy controls. Results of these studies revealed a marked association of Prevotella copri with new-onset RA (NORA) patients and not with other patient groups. Shotgun sequencing of the microbiome indicated that some P. copri genes are differentially present in NORA-associated and healthy samples. Colonization of mice with P. copri enhanced susceptibility to chemical colitis and collagen-induced arthritis, consistent with pro-inflammatory potential of this organism. Taken together, results presented herein demonstrate that NORA-associated P. copri contribute to the pathogenesis of human arthritis.

More particularly, high-throughput sequencing of the 16S gene (regions V1-V2, 454 platform) was performed on 114 fecal DNA samples [44 samples collected from NORA patients at the time of initial diagnosis and prior to immunosuppressive treatment, 26 samples from patients with chronic, treated rheumatoid arthritis (CRA), 16 samples from patients with psoriatic arthritis (PsA), and 28 samples from healthy controls (HLT)] to determine if particular bacterial clades are associated with rheumatoid arthritis. See Table 1 for additional details.

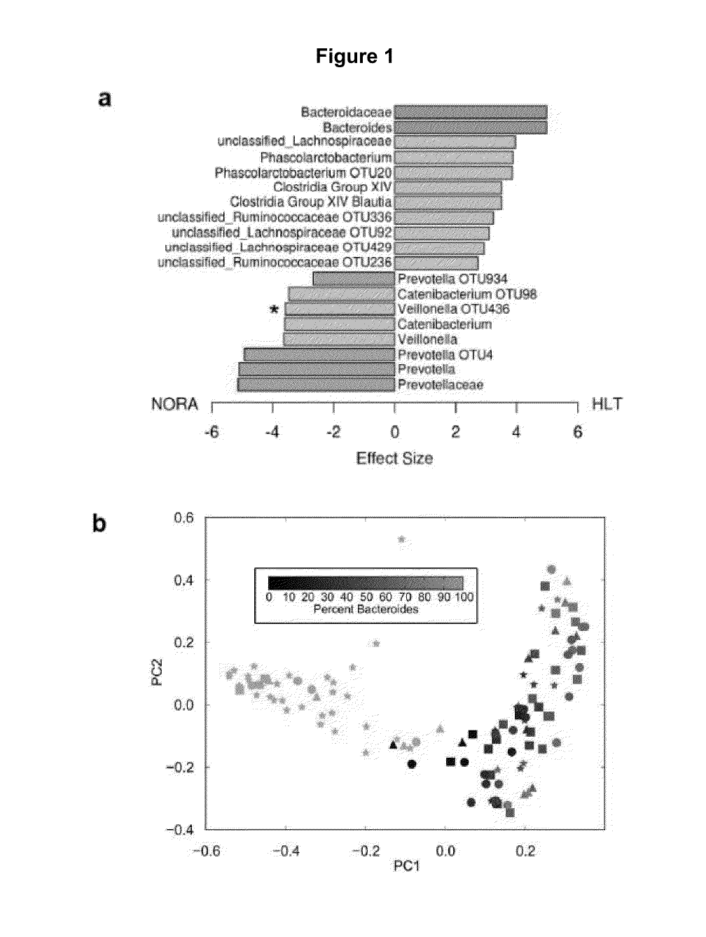

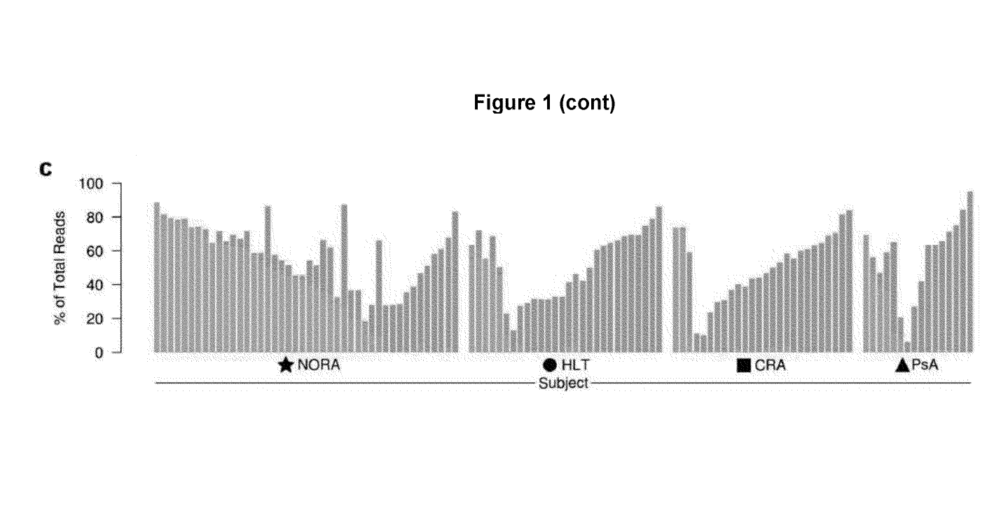

To determine if particular bacterial clades are associated with rheumatoid arthritis, sequences were analyzed with MOTHUR (Schloss et al., 2009) to cluster operational taxonomic units (OTUs, species level classification) at a 97% identity threshold, assign taxonomic identifiers, and calculate clade relative abundances. Although PsA patients revealed a reduction in sample diversity similar to that of IBD patients (Morgan et al., 2012), diversity was comparable between NORA, CRA and healthy groups at 3.02+/−0.66 (mean, SD) overall by Shannon Diversity Index (FIG. S1a). However, when applying Simpson's Dominance Index, the NORA group was less diverse (FIG. S1b), suggesting that these patients harbored a relatively higher abundance of common taxa. Analysis at the major taxonomic hierarchy levels showed no significant differences in either phyla abundance or the ratio of Bacteroidetes/Firmicutes (FIG. S1c) between all groups. At the level of family abundances, however, the present inventors noted a significant enrichment of Prevotellaceae in NORA subjects (FIG. 1a, S1d). Using the linear discriminant effect size method (LEfSe, see Methods) (Segata et al., 2011) to compare detected clades (33 families, 177 genera, 996 OTUs) among all groups, a positive association of two specific Prevotella OTUs with NORA and an inverse correlation with Group XIV Clostridia, Lachnospiraceae, and Bacteroides as compared to healthy controls (FIG. 1a) was found. Of all detected Prevotellaceae OTUs, OTU4 was the most highly represented with 171,486 supporting reads at 11.49+/−17.85 (mean, SD) percent of reads per sample. OTU12, the next most abundant Prevotellaceae, was supported by 12,119 reads at 2.00+/−5.42 (mean, SD) percent of reads per sample. Other Prevotellaceae OTUs (including Prevotella OTU934) were more scarcely represented with 1,232+/−2,305 (mean, SD) total supporting reads at less than 0.5% total reads per sample. The present inventors therefore reasoned that OTU4 was the dominant Prevotella in the cohort with 6-fold more supporting reads than the next most abundant OTU. Principal coordinate analysis with Bray-Curtis distances demonstrated that subjects form distinct clusters, irrespective of health or disease status (FIG. 1b). The largest component of microbial variation corresponded to the carriage (or absence) of Prevotella, which significantly differentiated NORA subjects from healthy controls and other forms of arthritis. Consistent with other reports of either high Prevotella or high Bacteroides relative abundance, but rarely a high relative abundance of both, (Faust et al., 2012, Yatsunenko et al., 2012), the present inventors found segregation of Prevotella or Bacteroides dominance in the intestinal microbiome (FIG. 1c).

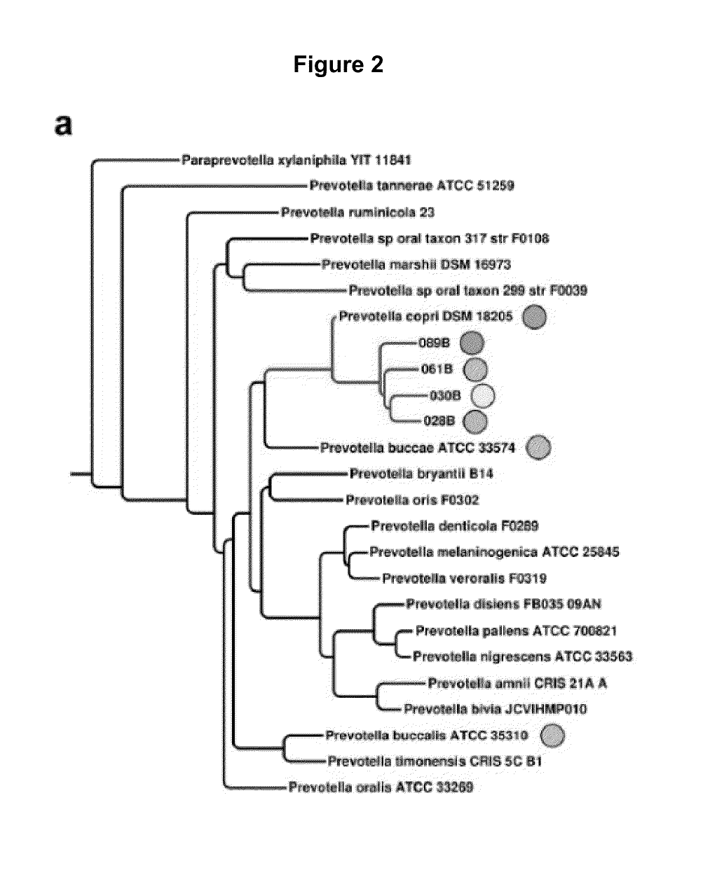

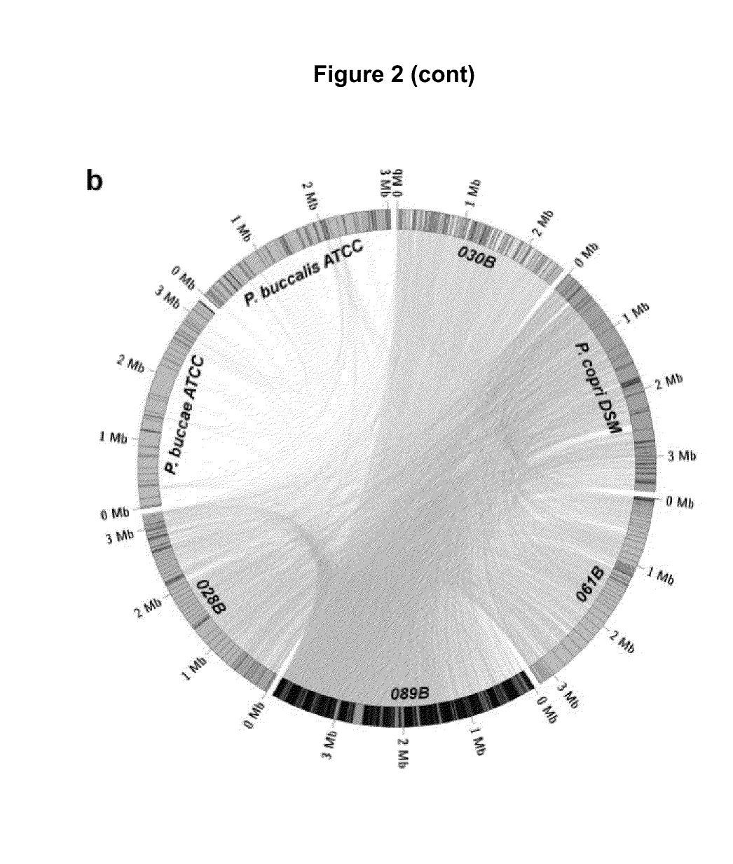

To taxonomically identify Prevotella OTU4, OTU12, and OTU934, a phylogenetic tree was generated using the consensus 16S sequences of these OTUs and matched regions from known Prevotella taxa (FIG. S2). The analysis revealed these OTUs to cluster tightly with Prevotella copri, a microbe isolated from human feces (Hayashi et al., 2007) and sequenced as part of the HMP's reference genome initiative. To further characterize Prevotella OTU4, the most abundant taxon, four high-abundance NORA samples (028B, 030B, 061B, and 089B) were selected for shotgun sequencing (single-end, 454 platform). The resulting long reads were used to generate metagenomic assemblies (Table S1, see Methods) which served as input to PhyloPhlAn (Segata et al., 2013). Briefly, PhyloPhlAn locates 400 ubiquitous bacterial genes in a given assembly by sequence alignment in amino acid space, then builds a tree by concatenating the most discriminative positions in each gene into a single long sequence and applying FastTree (Price et al., 2010), a standard tree reconstruction tool. This produced a phylogenomic tree placing the taxon most represented in each sample's metagenomic contigs (i.e. Prevotella OTU4) again in close association with Prevotella copri (FIG. 2a). The present inventors therefore chose to filter the resulting metagenomic assemblies by alignment to the P. copri reference genome to generate draft patient-derived genome assemblies (see Methods). Comparison of these draft assemblies to reference P. copri and to one another revealed a high degree of similarity, with possible genome rearrangements (FIG. 2b).

Overall, 75% (33/44) of the NORA patients and 21.4% (6/28) of the healthy controls carried Prevotella copri in their intestinal microbiota compared to 11.5% (3/26) and 37.5% (6/16) in CRA and PsA patients, respectively, at a threshold for presence of >5% relative abundance. The prevalence of Prevotella copri in NORA compared to CRA, PsA, and healthy controls was statistically significant by chi-squared test, but was not significant in pairwise comparisons of the latter three cohorts (Table S2).

Although initial shotgun sequencing of the patient-derived strains showed their similarity to P. copri, there were notable differences observed in assembled genomes upon comparison with the P. copri reference genome. This observation suggested that the presence or absence of particular genes in these strains might correlate with health or disease phenotypes in this cohort. To address this question, shotgun sequencing was performed on fecal DNA from NORA and healthy subjects, and the present inventors chose to compare Prevotella sequences from 18 NORA Prevotella-positive subjects, which allowed for a depth of at least 7 M Prevotella-aligned reads (paired-end, 100 nt, Illumina platform), to those of P. copri from 17 healthy subjects (including 15 from the HMP database and 2 HLT from our cohort) (Table S3). Samples sequenced to a depth of less than 7 M such reads were excluded (FIG. S3c), having insufficient depth for complete recovery of P. copri ORFs (see Methods).

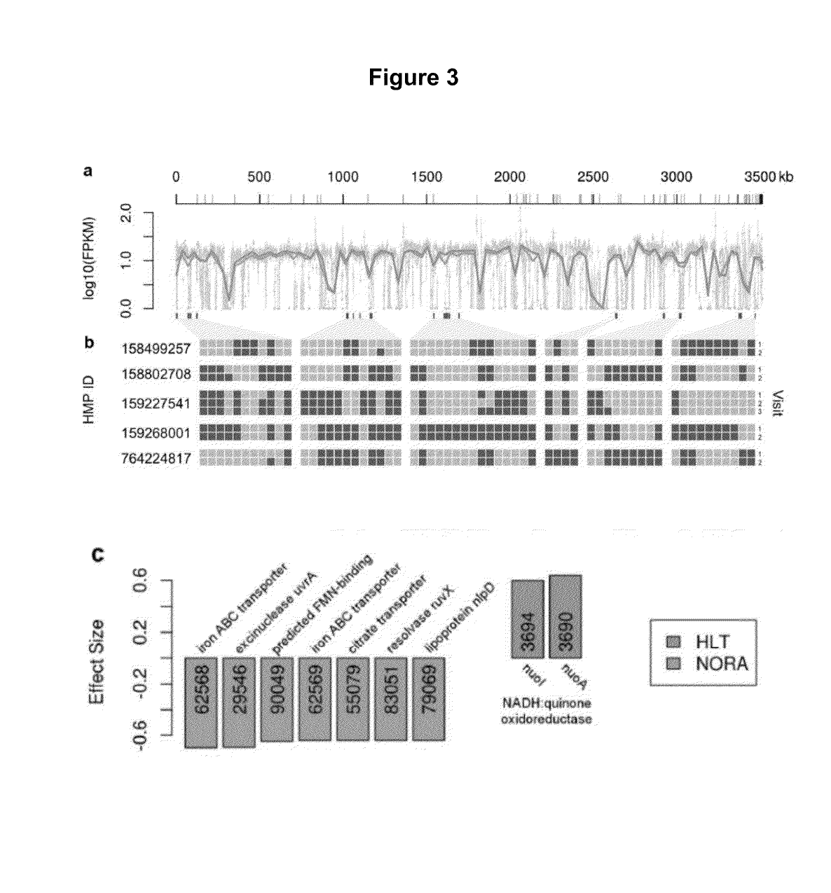

First, the present inventors examined the coverage of the P. copri reference genome by all subjects, as an indicator of inter-individual strain variability (HMP, 2012). Overall, coverage was similar between healthy and NORA subjects in all but a few regions (FIG. 3a, blue and red horizontal lines). Eight regions were poorly covered in all subjects with mean coverage below the 25^{th} percentile of 0.79 FPKM, while several regions showed substantial variability between individuals (FIG. 3a, gray vertical lines). To determine if the presence or absence of these regions within individuals was consistent between samplings, the present inventors applied MetaPhlAn (Segata et al., 2012) to Prevotella-positive HMP samples collected over multiple visits (FIG. 3b). Briefly, MetaPhlAn determines the presence or absence of metagenomic marker genes that are specific to particular bacterial clades by analyzing the coverage of such genes by sequenced reads. Genes are called specific for a bacterial clade if they are not found in any reference genomes outside the clade, but are found in all such genomes within the clade. In concordance with a previous report (Schloissnig et al., 2013) documenting the temporal stability of metagenomic SNP patterns in individuals, the present inventors found that carriage of P. copri genes within an individual varied little between samplings. In addition to a stable set of P. copri core marker genes common to all samples, a subset of variable marker genes was observed to co-occur in islands across the P. copri genome, suggesting genomic rearrangements as a mechanism of variability (FIG. 3a, blue boxes below plot). Together, these results suggest that P. copri strains vary between individuals and retain their individuality over time.

Next, the present inventors assembled a catalog of P. copri genes present across many individuals (i.e. the P. copri pangenome), by performing de novo meta-genome assembly and gene calling on a per-sample basis (see Methods). To determine if any ORFs were differentially present in NORA subjects as compared to healthy controls, the present inventors first reduced the set of interrogated ORFs by filtering partially assembled (i.e. containing gaps, lacking stop codons), short (i.e. less than 300 bp), and low-coverage (i.e. present in fewer than five subjects) ORFs to yield a final set of 3,291 high-confidence P. copri ORFs. (FIG. S3). The present inventors found two ORFs differentially present in healthy controls, and 17 ORFs differentially present in NORA (FIG. 3c and Table S4). The two healthy-specific ORFs appear on the same metagenomic contig, encoding a nearly-complete nuo operon for NADH:ubiquinone oxidoreductase (FIG. S4a), adjacent to a Bacteroides conjugative transposon. Similarly, two of the NORA-specific ORFs appear together on another metagenomic contig, encoding an ATP-binding cassette iron transporter (FIG. S4b). These ORFs may represent good biomarkers for discrimination between healthy and disease-associated microbiota in the population at risk for RA.

TABLE S4
Presence/absence, p-values and FDR statistics for differentially represented
ORFs in the P. copri pangenome biomarker analysis, with annotations.

| | HLT | HLT | NORA | NORA | | | | |
|---|---|---|---|---|---|---|---|---|
| ID | Present | Absent | Present | Absent | Effect Size | p-value | BH q-value | Annotation |
| gene_id_62568 | 0 | 16 | 11 | 7 | −0.69565217 | 0.000126502 | 0.235954574 | K02016 |
| gene_id_29546 | 8 | 8 | 18 | 0 | −0.69230769 | 0.000708849 | 0.235954574 | K03701 |
| gene_id_90049 | 6 | 10 | 17 | 1 | −0.64822134 | 0.00063033 | 0.235954574 | K07005 |
| gene_id_62569 | 0 | 16 | 9 | 9 | −0.64 | 0.001145063 | 0.235954574 | K02015 |
| gene_id_55079 | 0 | 16 | 9 | 9 | −0.64 | 0.001145063 | 0.235954574 | |
| gene_id_83051 | 0 | 16 | 9 | 9 | −0.64 | 0.001145063 | 0.235954574 | K07447 |
| gene_id_79069 | 0 | 16 | 9 | 9 | −0.64 | 0.001145063 | 0.235954574 | K06194 |
| gene_id_68986 | 1 | 15 | 12 | 6 | −0.63736263 | 0.000365213 | 0.235954574 | K07652 |
| gene_id_54057 | 1 | 15 | 12 | 6 | −0.63736263 | 0.000365213 | 0.235954574 | K06001 |
| gene_id_45456 | 2 | 14 | 13 | 5 | −0.60350877 | 0.000628132 | 0.235954574 | K00852 |
| gene_id_29407 | 1 | 15 | 11 | 7 | −0.59848484 | 0.001109123 | 0.235954574 | |
| gene_id_45366 | 1 | 15 | 11 | 7 | −0.59848484 | 0.001109123 | 0.235954574 | |
| gene_id_81143 | 1 | 15 | 11 | 7 | −0.59848484 | 0.001109123 | 0.235954574 | K01752 |
| gene_id_45134 | 1 | 15 | 11 | 7 | −0.59848484 | 0.001109123 | 0.235954574 | K00970 |
| gene_id_17194 | 4 | 12 | 15 | 3 | −0.58947368 | 0.001428318 | 0.247850777 | |
| gene_id_68779 | 4 | 12 | 15 | 3 | −0.58947368 | 0.001428318 | 0.247850777 | |
| gene_id_59356 | 4 | 12 | 15 | 3 | −0.58947368 | 0.001428318 | 0.247850777 | |
| gene_id_3694 | 15 | 1 | 7 | 11 | 0.598484848 | 0.001109123 | 0.235954574 | K00330 |
| gene_id_3690 | 15 | 1 | 6 | 12 | 0.637362637 | 0.000365213 | 0.235954574 | K00338 |

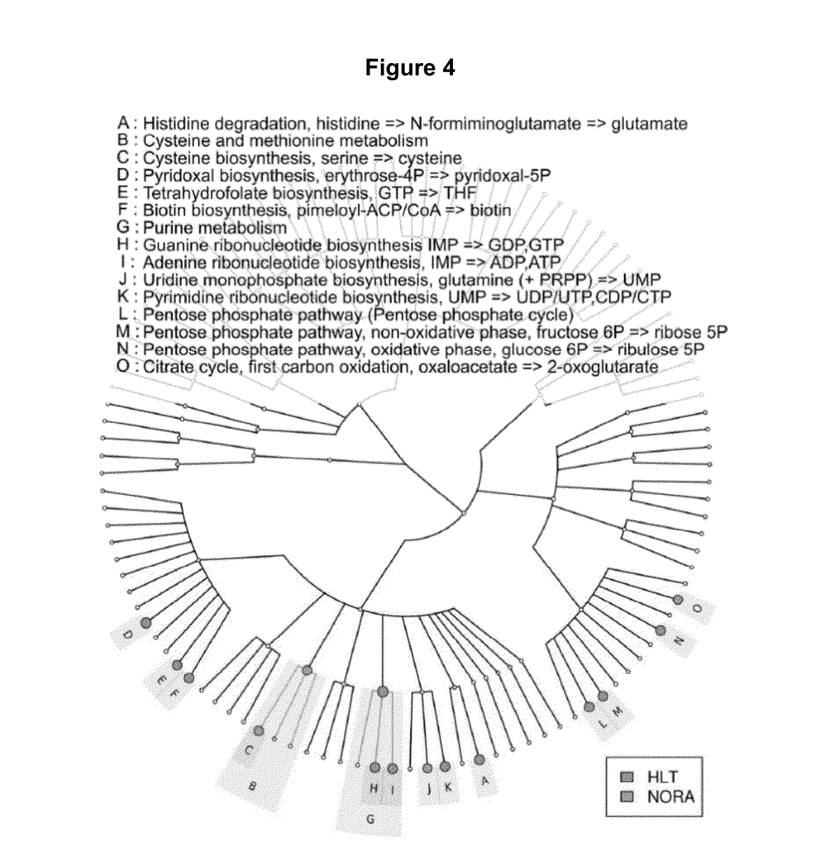

To determine if the NORA metagenome encodes unique functions compared to healthy subjects, the present inventors applied HUMAnN (Abubucker et al., 2012) to quantitate the coverage and abundances of KEGG (Kanehisa and Goto, 2000) modules (small sets of genes in well-defined metabolic pathways) in healthy controls (n=5) and a representative set of NORA subjects (n=14) with and without Prevotella. LEfSe (Segata et al., 2011) was then applied to find statistically significant differences between groups. This analysis revealed a low abundance of vitamin metabolism (i.e. biotin, pyroxidal, and folate) and pentose phosphate pathway modules in NORA, consistent with a lack of these functions in Prevotella genomes (FIG. 4). At the coverage level (presence or absence), the NORA metagenome is defined by an absence of functions present in Bacteroides and Clostridia, clades typically found in low abundance in Prevotella-high NORA subjects.

Prevotella and Bacteroides are closely related both functionally and phylogenetically, yet, surprisingly, are rarely found together in high relative abundance despite their ability to dominate the gut microbiome individually (Faust et al., 2012). The present inventors hypothesized that there might be a genetic difference in these two clades that could account for their apparent co-exclusionary relationship. The present inventors therefore sought to find genes differentially present in P. copri but not in any of the most abundant Bacteroides species. This revealed K05919 (superoxide reductase), K00390 (phosphoadenosine phosphosulfate reductase), and several transporters as uniquely present in P. copri (Table S5), and also a set of genes absent in P. copri but present in Bacteroides (Table S6).

TABLE S5
KOs present in Prevotella copri DSM 18205 but not in any
Bacteroides accounting for at least 5% of the total microbiota
in any subject of the Human Microbiome Project.

| KO | Description |
|---|---|
| K00040 | fructuronate reductase [EC: 1.1.1.57] |
| K00390 | phosphoadenosine phosphosulfate reductase [EC: 1.8.4.8] |
| K00662 | aminoglycoside N3′-acetyltransferase [EC: 2.3.1.81] |
| K00878 | hydroxyethylthiazole kinase [EC: 2.7.1.50] |
| K01259 | proline iminopeptidase [EC: 3.4.11.5] |
| K01267 | aspartyl aminopeptidase [EC: 3.4.11.21] |
| K03289 | MFS transporter, NHS family, nucleoside permease |
| K03549 | KUP system potassium uptake protein |
| K03579 | ATP-dependent helicase HrpB [EC: 3.6.4.13] |
| K05794 | tellurite resistance protein TerC |
| K05919 | superoxide reductase [EC: 1.15.1.2] |
| K06215 | pyridoxine biosynthesis protein [EC: 4.—.—.—] |
| K06987 | Unclassified; Poorly Characterized; General function |
| | prediction only |
| K07007 | Unclassified; Poorly Characterized; General function |
| | prediction only |
| K07074 | Unclassified; Poorly Characterized; General function |
| | prediction only |
| K07090 | Unclassified; Poorly Characterized; General function |
| | prediction only |
| K07487 | transposase |
| K08234 | glyoxylase I family protein |
| K08681 | glutamine amidotransferase [EC: 2.6.—.—] |
| K08714 | voltage-gated sodium channel |
| K08884 | serine/threonine protein kinase, bacterial [EC: 2.7.11.1] |
| K09144 | hypothetical protein |
| K09802 | hypothetical protein |

TABLE S6
KOs present in all genomes available for Bacteroides accounting
for at least 5% of the total microbiota in any subject of the Human
Microbiome Project and not present in Prevotella copri DSM 18205.

| KO | Description |
|---|---|
| K01079 | phosphoserine phosphatase [EC: 3.1.3.3] |
| K03771 | peptidyl-prolyl cis-trans isomerase SurA [EC: 5.2.1.8] |
| K02371 | enoyl-[acyl carrier protein] reductase II [EC: 1.3.1.—] |
| K01155 | type II restriction enzyme [EC: 3.1.21.4] |
| K02117 | V-type H+-transporting ATPase subunit A [EC: 3.6.3.14] |
| K09117 | hypothetical protein |
| K02112 | F-type H+-transporting ATPase subunit beta [EC: 3.6.3.14] |
| K11537 | MFS transporter, NHS family, xanthosine permease |
| K01507 | inorganic pyrophosphatase [EC: 3.6.1.1] |
| K02118 | V-type H+-transporting ATPase subunit B [EC: 3.6.3.14] |
| K03442 | small conductance mechanosensitive channel |
| K12373 | hexosaminidase [EC: 3.2.1.52] |
| K01077 | alkaline phosphatase [EC: 3.1.3.1] |
| K01805 | xylose isomerase [EC: 5.3.1.5] |
| K03118 | sec-independent protein translocase protein TatC |
| K00605 | aminomethyltransferase [EC: 2.1.2.10] |
| K07322 | regulator of cell morphogenesis and NO signaling |
| K01447 | N-acetylmuramoyl-L-alanine amidase [EC: 3.5.1.28] |
| K00957 | sulfate adenylyltransferase subunit 2 [EC: 2.7.7.4] |
| K00956 | sulfate adenylyltransferase subunit 1 [EC: 2.7.7.4] |
| K13694 | lipoprotein Spr |
| K01847 | methylmalonyl-CoA mutase [EC: 5.4.99.2] |
| K00077 | 2-dehydropantoate 2-reductase [EC: 1.1.1.169] |
| K01187 | alpha-glucosidase [EC: 3.2.1.20] |
| K01689 | enolase [EC: 4.2.1.11] |
| K02437 | glycine cleavage system H protein |
| K03644 | lipoic acid synthetase [EC: 2.8.1.8] |
| K03474 | pyridoxine 5-phosphate synthase [EC: 2.6.99.2] |
| K01041 | Unclassified; Poorly Characterized; General function |
| | prediction only |
| K07170 | GAF domain-containing protein |
| K01992 | ABC-2 type transport system permease protein |
| K01206 | alpha-L-fucosidase [EC: 3.2.1.51] |
| K11070 | spermidine/putrescine transport system permease protein |
| K11072 | spermidine/putrescine transport system ATP-binding protein |
| | [EC: 3.6.3.31] |
| K03801 | lipoyl(octanoyl) transferase [EC: 2.3.1.181] |
| K03559 | biopolymer transport protein ExbD |
| K07588 | LAO/AO transport system kinase [EC: 2.7.—.—] |
| K06973 | Unclassified; Poorly Characterized; General function |
| | prediction only |
| K01163 | hypothetical protein |
| K01759 | lactoylglutathione lyase [EC: 4.4.1.5] |
| K01624 | fructose-bisphosphate aldolase, class II [EC: 4.1.2.13] |
| K03113 | translation initiation factor 1 |
| K02123 | V-type H+-transporting ATPase subunit I [EC: 3.6.3.14] |
| K05595 | multiple antibiotic resistance protein |
| K00634 | phosphate butyryltransferase [EC: 2.3.1.19] |
| K00860 | adenylylsulfate kinase [EC: 2.7.1.25] |
| K01241 | AMP nucleosidase [EC: 3.2.2.4] |
| K02124 | V-type H+-transporting ATPase subunit K [EC: 3.6.3.14] |
| K01092 | myo-inositol-1(or 4)-monophosphatase [EC: 3.1.3.25] |
| K02481 | two-component system, NtrC family, response regulator |
| K03976 | putative transcription regulator |
| K01126 | glycerophosphoryl diester phosphodiesterase [EC: 3.1.4.46] |
| K08218 | MFS transporter, PAT family, beta-lactamase induction |
| | signal transducer AmpG |
| K10947 | PadR family transcriptionai regulator, regulatory protein |
| | PadR |
| K02078 | acyl carrier protein |
| K03699 | putative hemolysin |
| K00651 | homoserine O-succinyltransferase [EC: 2.3.1.46] |
| K03525 | type III pantothenate kinase [EC: 2.7.1.33] |
| K07043 | Unclassified; Poorly Characterized; General function |
| | prediction only |
| K08590 | carbon-nitrogen hydrolase family protein |
| K01573 | oxaloacetate decarboxylase, gamma subunit [EC: 4.1.1.3] |
| K00937 | polyphosphate kinase [EC: 2.7.4.1] |

In accordance with these findings, the present inventors have established a correlation between NORA and increased expression or the presence of ORFs as set forth in Table S4 (SEQ ID NOs: 1-17) and FIG. S4b; and the presence of KOs as set forth in Table S5. The present inventors have, moreover, established an inverse or negative correlation between NORA and increased expression or the presence of ORFs as set forth in Table S4 (SEQ ID NOs: 18-19) and FIG. S4a; and the presence of KOs as set forth in Table S6. Accordingly, detection of these ORFs and KOs can be used to diagnose NORA in a subject or evaluate the predisposition for a subject to be afflicted with NORA. The present inventors have, therefore, established a diagnostic signature characteristic of NORA and/or determinative for NORA risk.

In view of the results presented in Table S4, for example, detection of the presence of any one of or at least one of SEQ ID NOs: 1, 4, 5, 6, or 7 in a biological sample isolated from a subject serves as a strong diagnostic biomarker/indicator for NORA and/or the likelihood that a subject will be afflicted with NORA. This is underscored by the fact that, at least in this sample population, none of the healthy subjects was positive for the presence of any one of SEQ ID NOs: 1, 4, 5, 6, or 7.

Along the same lines, the presence of any one of or at least one of SEQ ID NOs: 8, 9, 11, 12, 13, or 14 in a biological sample isolated from a subject also serves as a strong diagnostic biomarker/indicator for NORA and/or the likelihood that a subject will be afflicted with NORA. This is underscored by the fact that, at least in this sample population, only one out of 16 healthy subjects was positive for the presence of any one of SEQ ID NOs: 8, 9, 11, 12, 13, or 14.

The presence of any one of or at least one of SEQ ID NOs: 2, 3, 15, 16, or 17 in a biological sample also serves as a strong diagnostic biomarker/indicator for NORA and/or the likelihood that a subject will be afflicted with NORA. The significance of these NORA diagnostic biomarkers/indicators is evident from their high frequency in the NORA positive group analyzed. More specifically, all of the 18 NORA patients were positive for the presence of SEQ ID NO: 2; 17 of the 18 NORA patients were positive for the presence of SEQ ID NO: 3; and 15 of the 18 NORA patients were positive for the presence of any one of SEQ ID NOs: 15, 16, or 17.

Results presented in Table S4 also offer strong evidence that the presence of either of SEQ ID NO: 18 or 19 in a biological sample is a strong diagnostic biomarker/indicator that the subject from whom the sample was isolated is healthy and is not at risk for being afflicted by NORA. The fact that 15 out of 16 healthy subjects assessed were positive for either of SEQ ID NO: 18 or 19 highlights the significance of these ORFs.

Turning next to results presented in FIG. S4, examining the discriminative biomarker/indicator ORFs in a metagenomic context reveals that certain of the ORFs identified co-localize and encode different components contributing to common functionality. More particularly, ORFs 62568 (SEQ ID NO: 1) and 62569 (SEQ ID NO: 4) occur on the same contig, are NORA-specific, and encode components of the same iron ABC transporter. These findings suggest that iron transport contributes to or is involved in some manner with NORA. In contrast, two ORFs, 3690 (SEQ ID NO: 19) and 3694 (SEQ ID NO: 18), are healthy-specific, occur on the same contig, and encode different components of the same NADH:quinone oxidoreductase. These results suggest that this enzyme or a pathway in which it plays a role contributes to or is involved in some manner with a healthy state.

In a further aspect, diagnostic biomarkers/indicators described herein are also envisioned as therapeutic biomarkers/indicators. In that determining the presence and/or amount of one of the aforementioned biomarkers/indicators can be used for diagnosing NORA and/or predicting the likelihood that a subject will be afflicted with NORA, it is envisioned that determining the presence and/or amount of one of these biomarkers/indicators can also be used as a therapeutic indicator. It is to be understood that in such therapeutic embodiments, detection of the relevant biomarkers/indicators is performed before and after administration of the potential therapeutic compound for the purposes of comparison.

In a particular embodiment, detection of the presence of or an increase in an ORF positively correlated with NORA (a NORA-specific open reading frame; SEQ ID NOs: 1-17; FIG. S4b) or a KO positively correlated with P. copri (See Table S5) following treatment with a potential therapeutic compound would indicate that the therapeutic compound is not efficacious. Under such a circumstance, the presence of, for example, a NORA-specific ORF following treatment as compared relative to the absence of the NORA-specific ORF prior to treatment indicates that the compound is not efficacious. Likewise, an increase in a NORA-specific ORF following treatment as compared relative to that detected prior to treatment indicates that the compound is not efficacious.

In another particular embodiment, detection of the presence of or an increase in a healthy-specific open reading frame (SEQ ID NOs: 18 and 19; FIG. S4a) or a KO positively correlated with Bacteroides (See Table S6) following treatment with a potential therapeutic compound would indicate that the therapeutic compound is efficacious. Under such a circumstance, the presence of, for example, a healthy-specific ORF following treatment as compared relative to the absence of the healthy-specific ORF prior to treatment indicates that the therapeutic compound is efficacious. Likewise, an increase in a healthy-specific ORF following treatment as compared relative to that detected prior to treatment indicates that the therapeutic compound is efficacious.

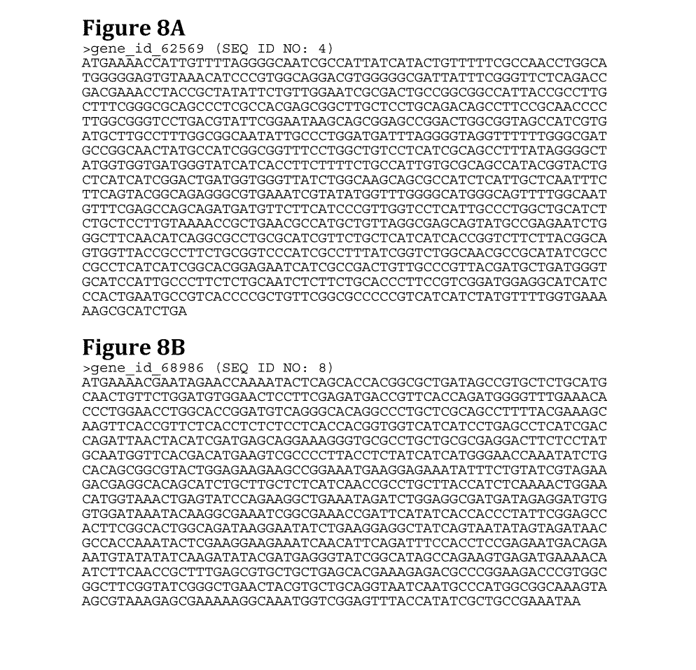

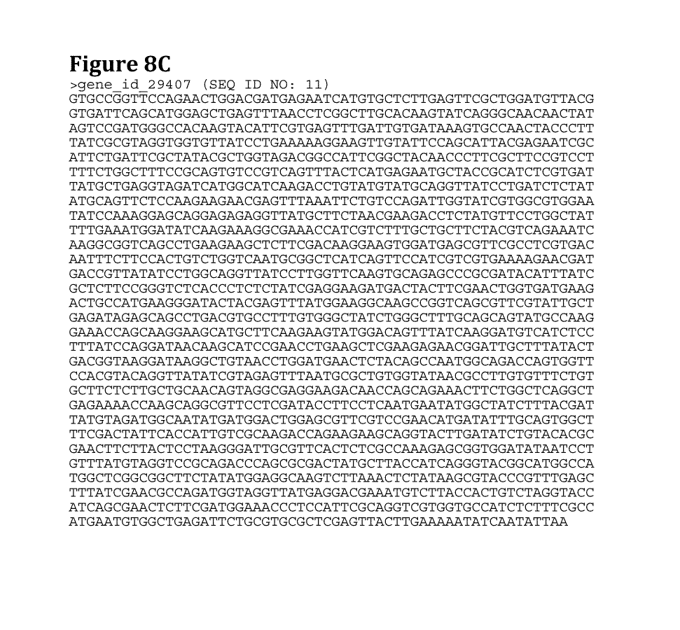

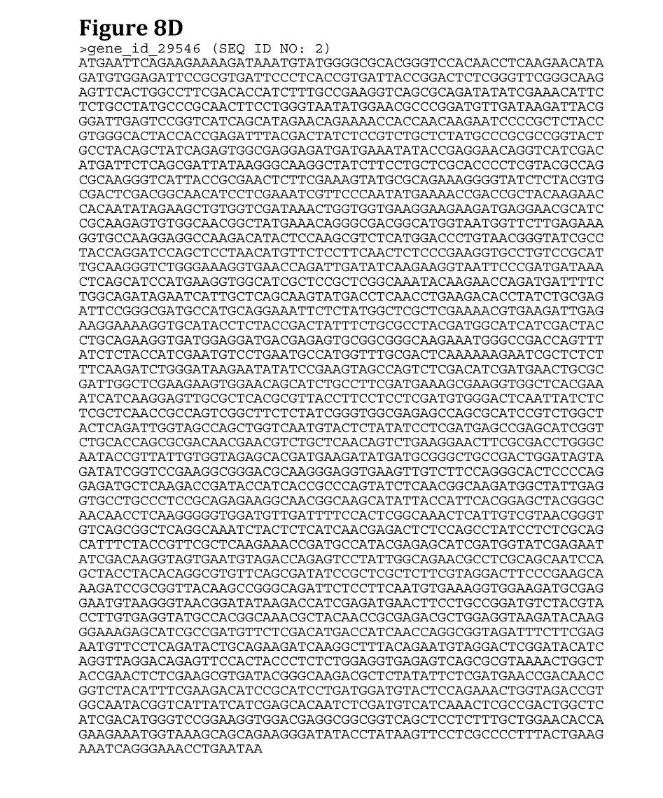

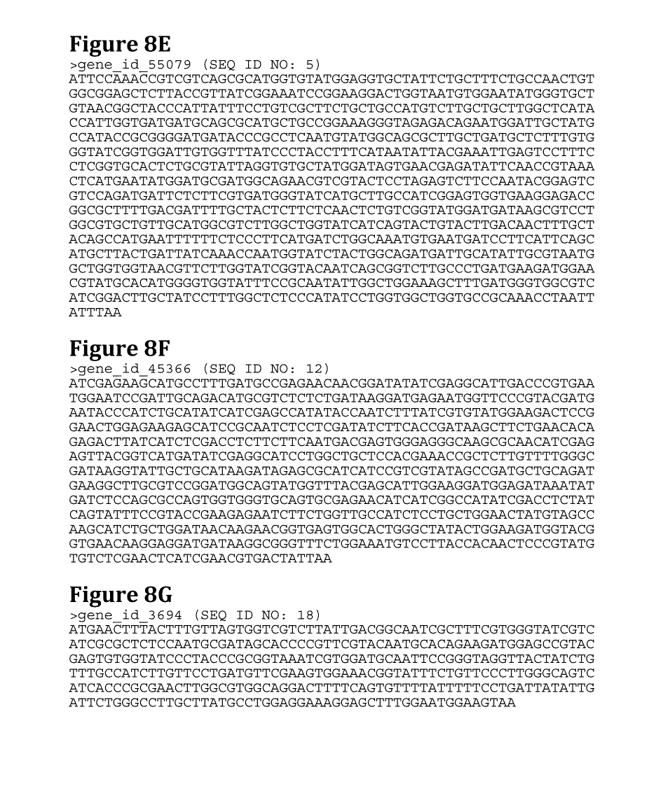

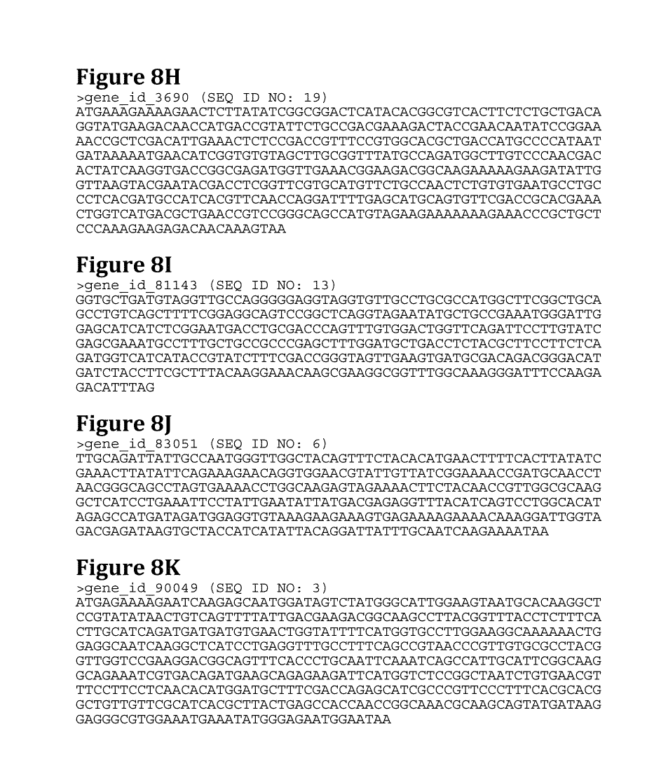

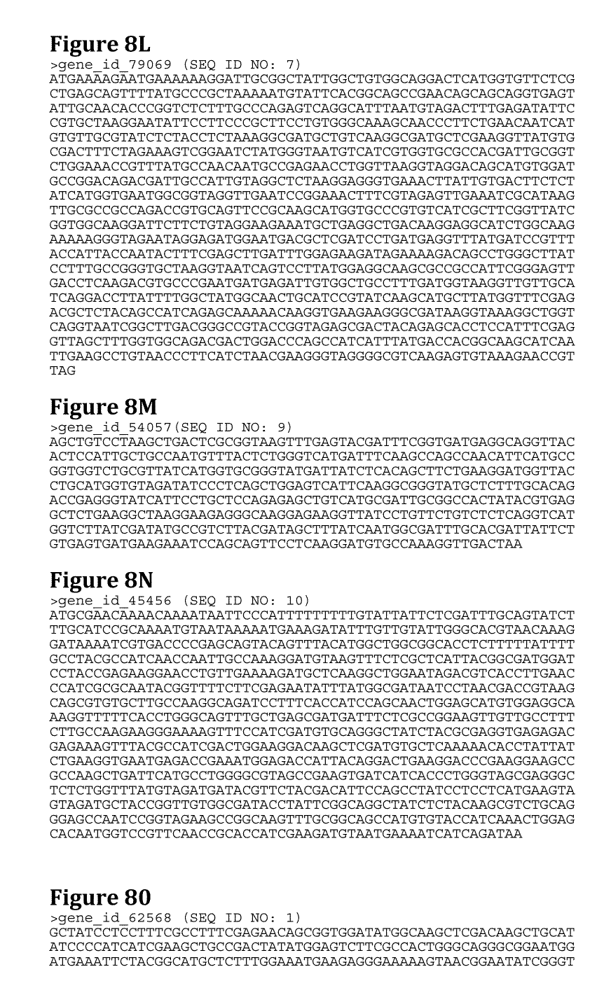

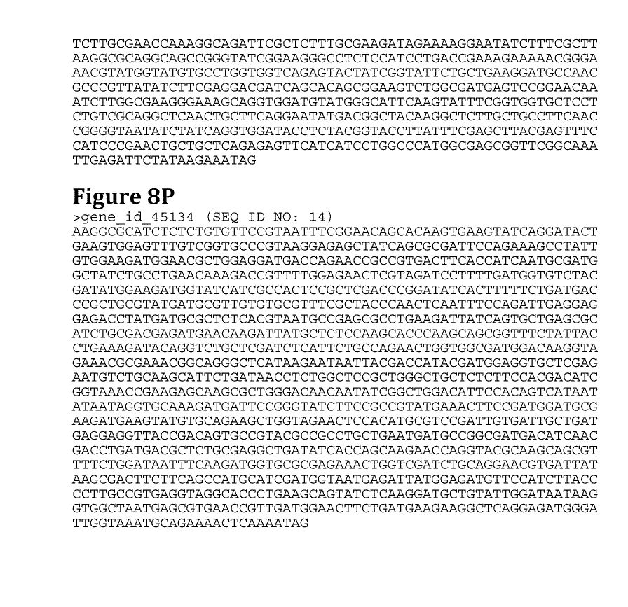

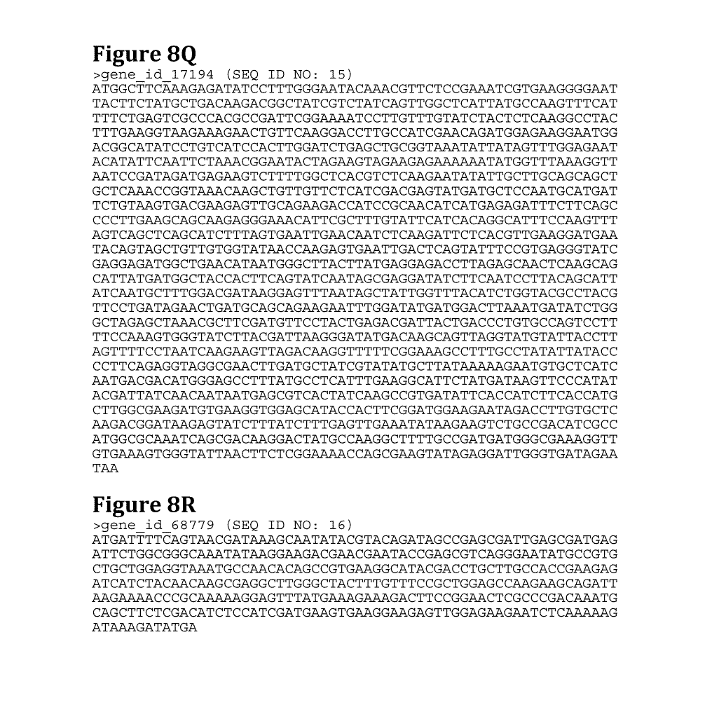

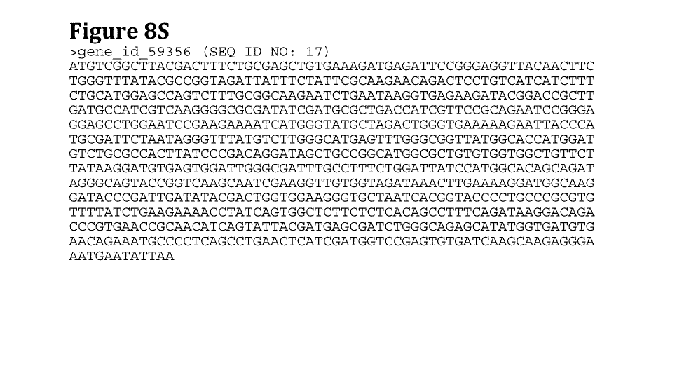

The identification of a panel of biomarkers/indicators for early disease as set forth herein and methods for using same makes available a straightforward assay whereby a stool sample can be used to identify subjects/patients at-risk for RA development and in the early phases of disease, so therapy can be instituted and tissue damage, deformity and disability can potentially be prevented. The biomarkers described herein, for example, nucleic acid sequences comprising any one of SEQ ID NOs: 1-17, detection of which serves as an indicator of P. copri, can be used alone or in combination with others biomarkers for RA or new-onset RA. Absence of nucleic acid sequences comprising either one of SEQ ID NOs: 18 or 19 can also be used as a biomarker for RA or new-onset RA, either alone or in combination with others biomarkers of RA or new-onset RA. As further described herein, detection of the presence or absence of, for example, any one of SEQ ID NOs: 1-19 also provides tools/methods for evaluating efficacy of a therapeutic regimen in an ongoing basis. Nucleic acid sequences corresponding to SEQ ID NOs: 1-17 are presented in FIG. 8.

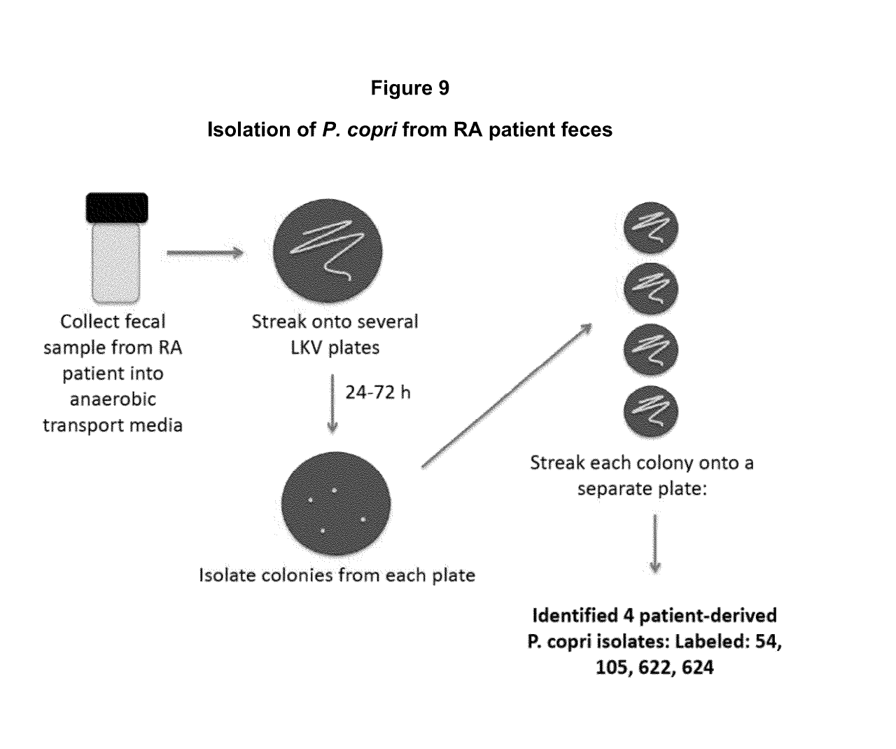

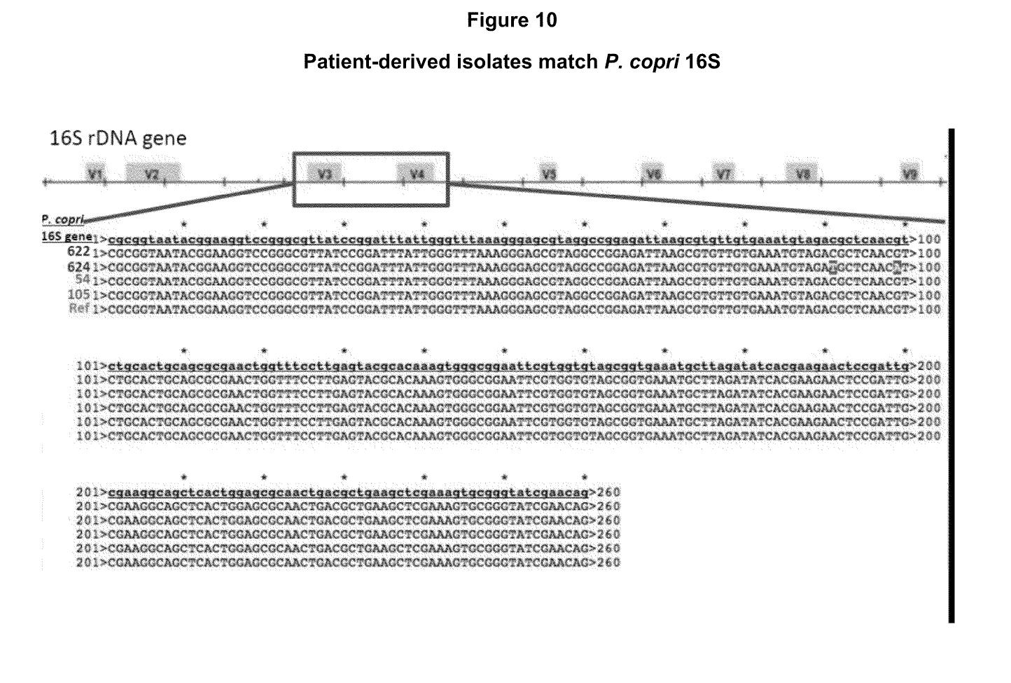

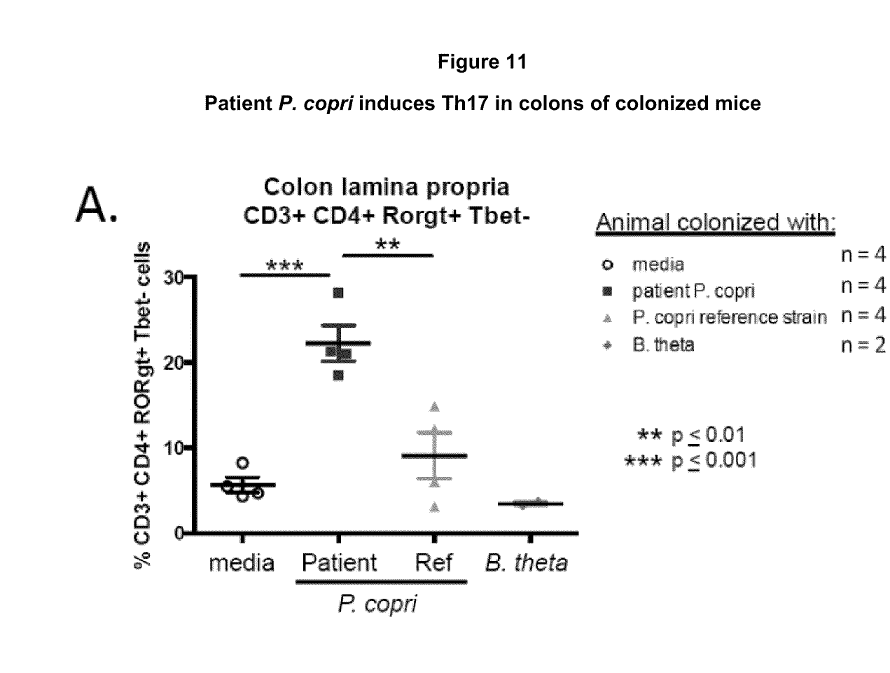

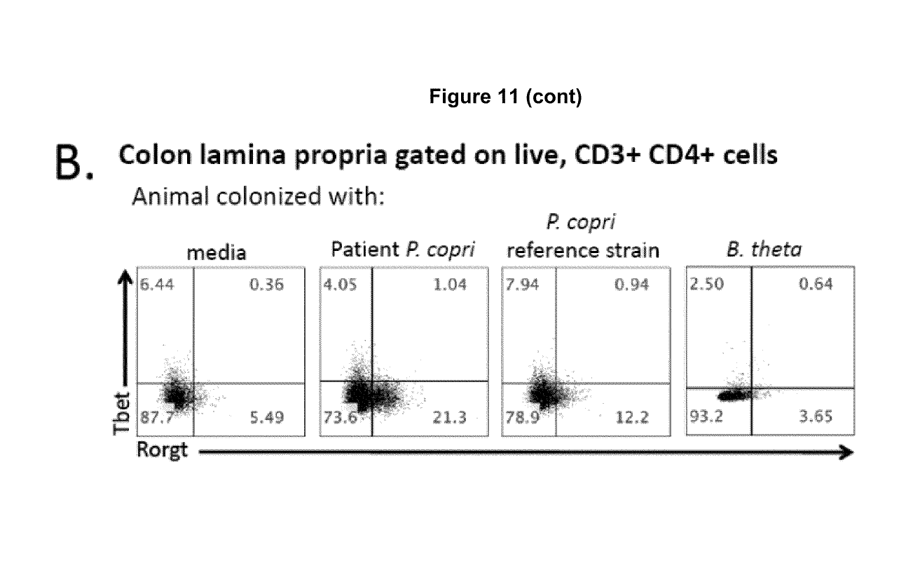

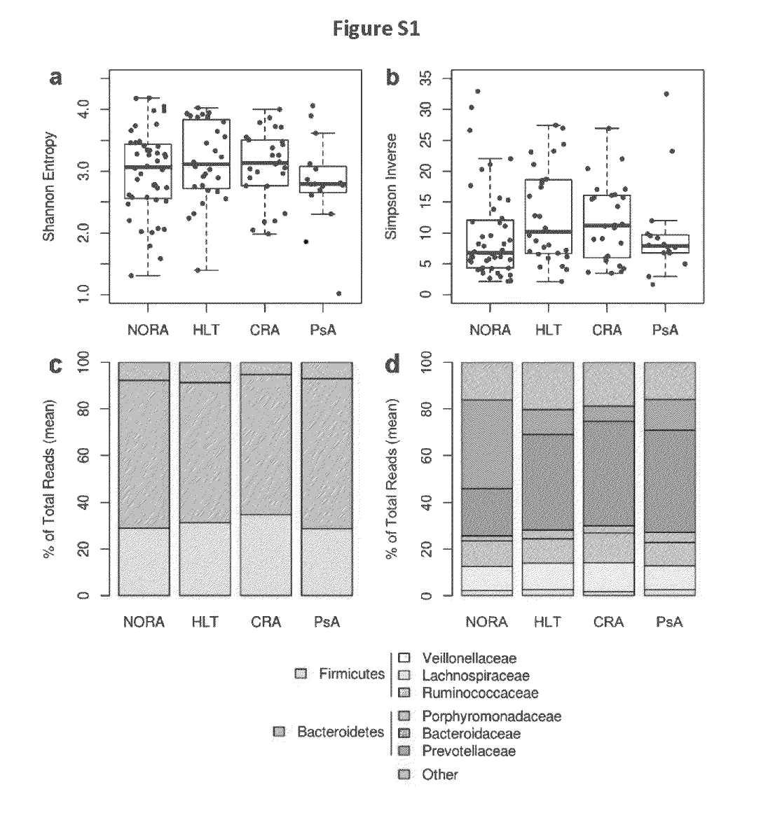

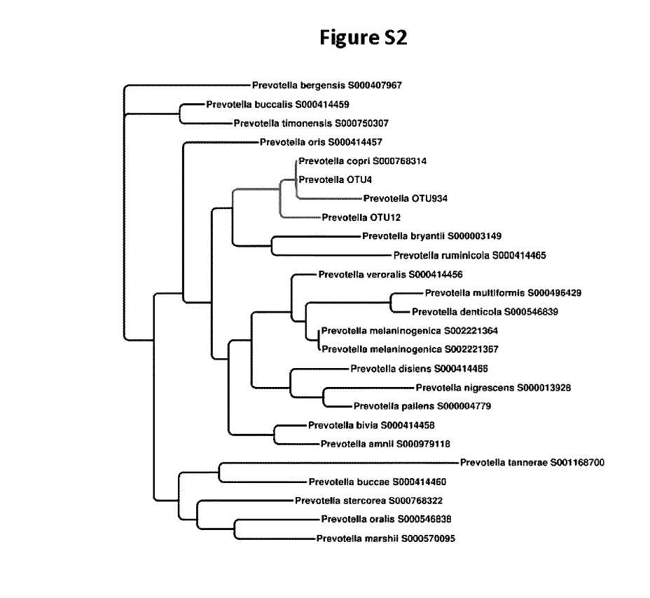

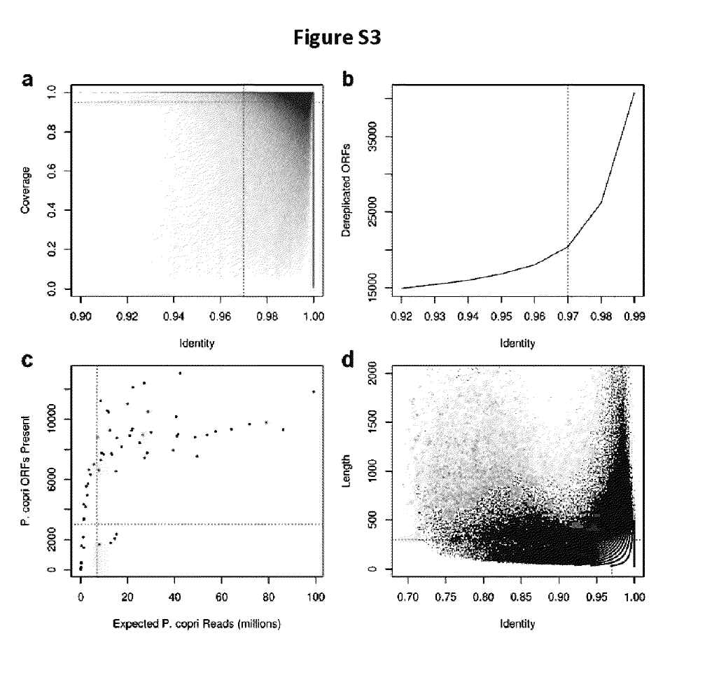

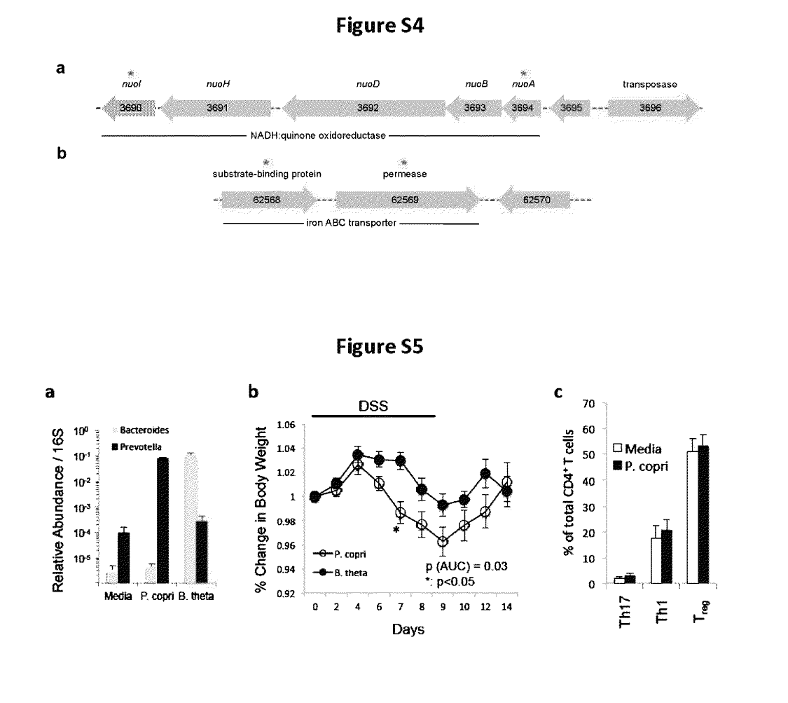

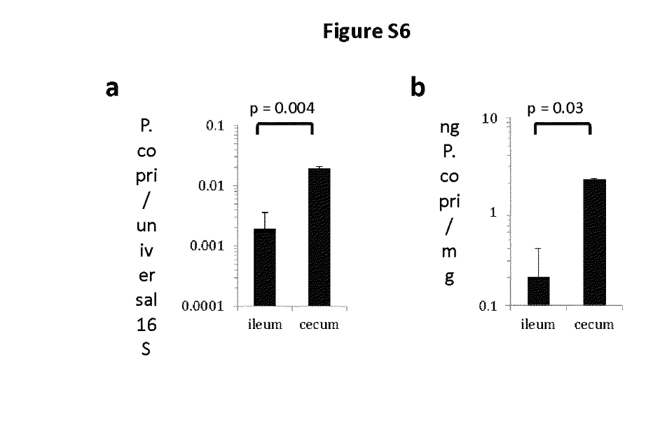

To investigate further the role of P. copri in RA, fecal samples were collected from RA patients into anaerobic transport media and subsequently streaked onto LKV plates. After incubating the plates under growth favorable conditions, single bacterial colonies were isolated from each plate streaked onto individual plates. See FIG. 9. Nucleic acid sequence analysis of the V3-V5 16S regions of four P. copri isolates (54, 105, 622, 624) from two rheumatoid arthritis patients revealed that the isolates are greater than 97% similar to reference P. copri 16S, meeting the definition of the same OTU. See FIG. 10 and Table 2. Preliminary analysis of two P. copri genomes from one patient reveals that 89% of 250 bp reads from the patient-derived genomes are greater than 95% similar to the draft reference genome of P. copri. See FIG. 10 and Table 3. Additional experiments revealed that patient-derived P. copri (isolate 624) induces local Th17 differentiation in the colon lamina propria, indicating a local T cell response to colonization. See FIG. 11. This response is observed only in mice colonized with patient P. copri, not reference P. copri (DSMZ, CB7), suggesting a functional role for differences between these two genomes.

TABLE 2
Percent identity of V3-V5 16S regions of RA patient fecal isolates to
reference P. copri. Patient-derived isolates match P. copri.

| Strain | Percent identity |
|---|---|
| 622 | 100 |
| 624 | 99 |
| 54 | 100 |
| 105 | 100 |
| P. copri reference strain | 100 |

TABLE 3
Similarity of patient P. copri isolate genomes
to reference P. copri draft genome. Isolates 622 and
624 compared to P. copri reference genome

| | P. copri strains sequenced | | |
|---|---|---|---|
| | Patient | Patient | P. copri |
| Align sequenced genome to | strain 624 | strain 622 | reference strain |
| P. copri reference | 89.5 | 89.6 | 99.2 |
| draft genome (Wash U) | | | |
| Bacteroides thetaiotaomicron | 53.1 | 53.6 | 56 |
| (YCH46) | | | |

As detailed herein, there is a need for improved methods for determining RA risk, particularly in those patients with a familial history of RA. There is, moreover, a need for diagnostic tools with which skilled practitioners can monitor asymptomatic, high risk patients using minimally invasive techniques to assess, on an ongoing basis, risk of RA onset. Improved diagnostic tools with which skilled practitioners can determine how best to treat a patient diagnosed with RA are also sought. These tools can, furthermore, be applied to methods for assessing if a therapeutic regimen is efficacious for the patient. The discoveries described herein address the above-indicated long sought diagnostic, prognostic, and therapeutic needs.

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

Therefore, if appearing herein, the following terms shall have the definitions set out below.

An “antibody” is any immunoglobulin, including antibodies and fragments thereof, that binds a specific epitope. The term encompasses polyclonal, monoclonal, and chimeric antibodies, the last mentioned described in further detail in U.S. Pat. Nos. 4,816,397 and 4,816,567.

An “antibody combining site” is that structural portion of an antibody molecule comprised of heavy and light chain variable and hypervariable regions that specifically binds antigen.

The phrase “antibody molecule” in its various grammatical forms as used herein contemplates both an intact immunoglobulin molecule and an immunologically active portion of an immunoglobulin molecule.

Exemplary antibody molecules are intact immunoglobulin molecules, substantially intact immunoglobulin molecules and those portions of an immunoglobulin molecule that contains the paratope, including those portions known in the art as Fab, Fab′, F(ab′)₂ and F(v), which portions are preferred for use in the therapeutic methods described herein.

Fab and F(ab′)₂ portions of antibody molecules are prepared by the proteolytic reaction of papain and pepsin, respectively, on substantially intact antibody molecules by methods that are well-known. See for example, U.S. Pat. No. 4,342,566 to Theofilopolous et al. Fab′ antibody molecule portions are also well-known and are produced from F(ab′)₂ portions followed by reduction of the disulfide bonds linking the two heavy chain portions as with mercaptoethanol, and followed by alkylation of the resulting protein mercaptan with a reagent such as iodoacetamide. An antibody containing intact antibody molecules is preferred herein.

The phrase “monoclonal antibody” in its various grammatical forms refers to an antibody having only one species of antibody combining site capable of immunoreacting with a particular antigen. A monoclonal antibody thus typically displays a single binding affinity for any antigen with which it immunoreacts. A monoclonal antibody may therefore contain an antibody molecule having a plurality of antibody combining sites, each immunospecific for a different antigen; e.g., a bispecific (chimeric) monoclonal antibody.

The subject or patient is preferably an animal, including but not limited to animals such as mice, rats, cows, pigs, horses, chickens, cats, dogs, etc., and is preferably a mammal, more preferably a primate, and most preferably a human.

The term “preventing” or “prevention” refers to a reduction in risk of acquiring or developing a disease or disorder (i.e., causing at least one of the clinical symptoms of the disease not to develop in a subject that may be exposed to a disease-causing agent, or predisposed to the disease in advance of disease onset).

The term “prophylaxis” is related to “prevention” and refers to a measure or procedure the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; and the administration of an anti-malarial agent such as chloroquine, in advance of a visit to a geographical region where malaria is endemic or the risk of contracting malaria is high.

The term “treating” or “treatment” of any disease or disorder refers, in one embodiment, to ameliorating the disease or disorder (i.e., arresting the disease or reducing the manifestation, extent or severity of at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In a further embodiment, “treating” or “treatment” relates to slowing the progression of the disease.

As used herein, the term new-onset rheumatoid arthritis (NORA) patient refers to any patient who fulfills 1987 ARA criteria and/or 2010 ACR/EULAR criteria for Rheumatoid Arthritis. Patients must have been recently diagnosed (less than six months of symptoms) and never treated with steroids or DMARDs. The exclusion criteria are, moreover, set forth in Example 1 below.

As used herein, the term “immune response” signifies any reaction produced by an antigen, such as a protein antigen, in a host having a functioning immune system. Immune responses may be either humoral, involving production of immunoglobulins or antibodies, or cellular, involving various types of B and T lymphocytes, dendritic cells, macrophages, antigen presenting cells and the like, or both. Immune responses may also involve the production or elaboration of various effector molecules such as cytokines, lymphokines and the like. Immune responses may be measured both in in vitro and in various cellular or animal systems.

An “immunological response” to a composition or vaccine comprised of an antigen is the development in the host of a cellular- and/or antibody-mediated immune response to the composition or vaccine of interest. Usually, such a response consists of the subject producing antibodies, B cells, helper T cells, suppressor T cells, and/or cytotoxic T cells directed specifically to an antigen or antigens included in the composition or vaccine of interest.

The phrase “pharmaceutically acceptable” refers to molecular entities and compositions that are physiologically tolerable and do not typically produce an allergic or similar untoward reaction, such as gastric upset, dizziness and the like, when administered to a human.

The phrase “therapeutically effective amount” is used herein to mean an amount sufficient to preferably reduce by at least about 30 percent, more preferably by at least 50 percent, most preferably by at least 90 percent, a clinically significant change in a pathological feature of a disease or condition.

Compositions containing molecules or compounds described herein can be administered for diagnostic and/or therapeutic treatments. In therapeutic applications, compositions are administered to a patient already suffering from RA, for example, in an amount sufficient to at least partially arrest the symptoms of the disease and its complications. An amount adequate to accomplish this is defined as a “therapeutically effective amount or dose.” Amounts effective for this use will depend on the severity of the disease and the weight and general state of the patient.

Compounds, such as antibiotics (e.g., vancomycin), for use in treating RA may be prepared in pharmaceutical compositions, with a suitable carrier and at a strength effective for administration by various means to a patient experiencing an adverse medical condition associated with NORA, wherein the presence or an increase in any one of SEQ ID NOs: 1-17 is detected, for the treatment thereof. A variety of administrative techniques may be utilized, among them parenteral techniques such as subcutaneous, intravenous and intraperitoneal injections, catheterizations and the like. Average quantities of the compounds or derivatives thereof may vary and in particular should be based upon the recommendations and prescription of a qualified physician or veterinarian.

Antibodies including both polyclonal and monoclonal antibodies may, moreover, possess certain diagnostic and/or therapeutic applications. For example, a NORA-specific ORF or a KO present in P. copri (See Tables S4 and S5) may encode a protein that is presented on the surface of P. copri and thus may serve as an antigen against which polyclonal and/or monoclonal antibodies can be generated by known techniques such as the hybridoma technique utilizing, for example, fused mouse spleen lymphocytes and myeloma cells. Likewise, small molecules that mimic or antagonize the activity(ies) of a NORA-specific ORF or a KO present in P. copri (See Tables S4 and S5) or a protein encoded thereby may be discovered or synthesized, and may be used in diagnostic and/or therapeutic protocols.

It will also be apparent based on results presented herein that a protein encoded by a NORA-specific ORF or a KO present in P. copri (See Tables S4 and S5) that is presented on the surface of P. copri may serve as an immunogen against which an immune response to P. copri in a subject in need thereof can be generated via immunization.

The general methodology for making monoclonal antibodies by hybridomas is well known. Immortal, antibody-producing cell lines can also be created by techniques other than fusion, such as direct transformation of B lymphocytes with oncogenic DNA, or transfection with Epstein-Barr virus. See, e.g., M. Schreier et al., “Hybridoma Techniques” (1980); Hammerling et al., “Monoclonal Antibodies And T-cell Hybridomas” (1981); Kennett et al., “Monoclonal Antibodies” (1980); see also U.S. Pat. Nos. 4,341,761; 4,399,121; 4,427,783; 4,444,887; 4,451,570; 4,466,917; 4,472,500; 4,491,632; 4,493,890.

Panels of monoclonal antibodies produced against a protein encoded by a NORA-specific ORF or a KO present in P. copri (See Tables S4 and S5) can be screened for various properties; i.e., isotype, epitope, affinity, etc. Such monoclonals can be readily identified in activity assays. High affinity antibodies are also useful for immunoaffinity purification purposes.

Further to the above, polyclonal or monoclonal antibodies are screened for their ability to bind to P. copri. Antibodies so identified have the potential to be used as therapeutics for the treatment of diseases/conditions, such as, for example, RA or NORA. Such antibodies can be used to target P. copri in a subject wherein there is an over-abundance of P. copri in the intestines to trigger antibody dependent cytolytic activity specifically against P. copri.

In a particular embodiment, an antibody produced against a protein encoded by a NORA-specific ORF or a KO present in P. copri (See Tables S4 and S5) is used in diagnostic methods or for therapeutic purposes. In a particular embodiment, an antibody produced against a protein encoded by a NORA-specific ORF or a KO present in P. copri (See Tables S4 and S5) is an affinity purified polyclonal antibody. In a more particular embodiment, the antibody is a monoclonal antibody (mAb). In an even more particular embodiment, the antibody produced against a protein encoded by a NORA-specific ORF or a KO present in P. copri (See Tables S4 and S5) is in the form of Fab, Fab′, F(ab′)₂ or F(v) portions of whole antibody molecules.

Methods for producing polyclonal anti-polypeptide antibodies are well-known in the art. See U.S. Pat. No. 4,493,795 to Nestor et al. A monoclonal antibody, typically containing Fab and/or F(ab′)₂ portions of useful antibody molecules, can be prepared using the hybridoma technology described in Antibodies—A Laboratory Manual, Harlow and Lane, eds., Cold Spring Harbor Laboratory, New York (1988), which is incorporated herein by reference.

A monoclonal antibody useful in practicing methods described herein can be produced by initiating a monoclonal hybridoma culture comprising a nutrient medium containing a hybridoma that secretes antibody molecules of the appropriate antigen specificity. The culture is maintained under conditions and for a time period sufficient for the hybridoma to secrete the antibody molecules into the medium. The antibody-containing medium is then collected. The antibody molecules can then be further isolated by well-known techniques.

Media useful for the preparation of these compositions are both well-known in the art and commercially available and include synthetic culture media, inbred mice and the like. An exemplary synthetic medium is Dulbecco's minimal essential medium (DMEM; Dulbecco et al., Virol. 8:396 (1959)) supplemented with 4.5 gm/l glucose, 20 mm glutamine, and 20% fetal calf serum. An exemplary inbred mouse strain is the Balb/c.

Methods for producing monoclonal antibodies are also well-known in the art. See Niman et al., Proc. Natl. Acad. Sci. USA, 80:4949-4953 (1983). Typically, an antigenic protein encoded by, for example, any one of SEQ ID NOs: 1-17 is used either alone or conjugated to an immunogenic carrier, as the immunogen. Hybridomas are screened for the ability to produce an antibody that immunoreacts with the particular immunogen used.

Also encompassed herein are therapeutic compositions useful for practicing the therapeutic methods described herein. A subject therapeutic composition may include, in admixture, a pharmaceutically acceptable excipient (carrier) and one or more of an agent (e.g., a small molecule inhibitor of P. copri specific protein encoded by a NORA specific ORF or a P. copri specific KO; a P. copri specific antibody generated using methods described herein; or an antibiotic or the like) that inhibits the proliferation and/or activity of P. copri, as described herein as an active ingredient.

The preparation of therapeutic compositions which contain polypeptides, analogs or active fragments as active ingredients is well understood in the art. Typically, such compositions are prepared as injectables, either as liquid solutions or suspensions, however, solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like and combinations thereof. In addition, if desired, the composition can contain minor amounts of auxiliary substances such as wetting or emulsifying agents, pH buffering agents which enhance the effectiveness of the active ingredient.

A polypeptide, analog or active fragment can be formulated into the therapeutic composition as neutralized pharmaceutically acceptable salt forms. Pharmaceutically acceptable salts include the acid addition salts (formed with the free amino groups of the polypeptide or antibody molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The therapeutic polypeptide-, analog- or active fragment-containing compositions are conventionally administered intravenously, as by injection of a unit dose, for example. The term “unit dose” when used in reference to a therapeutic composition of the present invention refers to physically discrete units suitable as unitary dosage for humans, each unit containing a predetermined quantity of active material calculated to produce the desired therapeutic effect in association with the required diluent; i.e., carrier, or vehicle.

The compositions are administered in a manner compatible with the dosage formulation, and in a therapeutically effective amount. The quantity to be administered depends on the subject to be treated, capacity of the subject's immune system to utilize the active ingredient, and degree of inhibition or cell modulation desired. Precise amounts of active ingredient required to be administered depend on the judgment of the practitioner and are peculiar to each individual. However, suitable dosages may range from about 0.1 to 20, preferably about 0.5 to about 10, and more preferably one to several, milligrams of active ingredient per kilogram body weight of individual per day and depend on the route of administration. Suitable regimes for initial administration and booster shots are also variable, but are typified by an initial administration followed by repeated doses at one or more hour intervals by a subsequent injection or other administration. Alternatively, continuous intravenous infusion sufficient to maintain concentrations often nanomolar to ten micromolar in the blood are contemplated.

A general method for site-specific incorporation of unnatural amino acids into proteins is described in Christopher J. Noren, Spencer J. Anthony-Cahill, Michael C. Griffith, Peter G. Schultz, Science, 244:182-188 (April 1989). This method may be used to create analogs with unnatural amino acids.

With respect to antibodies or binding partners or functional fragments thereof, the immunogen (e.g., a protein encoded by, for example, any one of SEQ ID NOs: 1-17) forms complexes with one or more antibody(ies) or binding partners and one member of the complex is labeled with a detectable label. The fact that a complex has formed and, if desired, the amount thereof, can be determined by known methods applicable to the detection of labels.

The labels most commonly employed for these studies are radioactive elements, enzymes, chemicals which fluoresce when exposed to ultraviolet light, and others.

A number of fluorescent materials are known and can be utilized as labels. These include, for example, fluorescein, rhodamine, auramine, Texas Red, AMCA blue and Lucifer Yellow. A particular detecting material is anti-rabbit antibody prepared in goats and conjugated with fluorescein through an isothiocyanate.

The antibodies or binding partners or functional fragments thereof specific for a protein encoded by, for example, any one of SEQ ID NOs: 1-17 can also be labeled with a radioactive element or with an enzyme. The radioactive label can be detected by any of the currently available counting procedures. The preferred isotope may be selected from ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re.

Enzyme labels are likewise useful, and can be detected by any of the presently utilized colorimetric, spectrophotometric, fluorospectrophotometric, amperometric or gasometric techniques. The enzyme is conjugated to the selected particle by reaction with bridging molecules such as carbodiimides, diisocyanates, glutaraldehyde and the like. Many enzymes which can be used in these procedures are known and can be utilized. The preferred are peroxidase, β-glucuronidase, β-D-glucosidase, β-D-galactosidase, urease, glucose oxidase plus peroxidase and alkaline phosphatase. U.S. Pat. Nos. 3,654,090; 3,850,752; and 4,016,043 are referred to by way of example for their disclosure of alternate labeling material and methods.

As used herein, the term “complementary” refers to two DNA strands that exhibit substantial normal base pairing characteristics. Complementary DNA may, however, contain one or more mismatches.

The term “hybridization” refers to the hydrogen bonding that occurs between two complementary DNA strands.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism. In a particular embodiment, the isolated nucleic acid sequence is a cDNA. In a more particular embodiment, the isolated nucleic acid sequence is a cDNA corresponding to, for example, any one of SEQ ID NOs: 1-19.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it is generally associated in its natural state (i.e., in cells or tissues). An isolated nucleic acid (either DNA or RNA) may further represent a molecule produced directly by biological or synthetic means and separated from other components present during its production.

“Natural allelic variants”, “mutants” and “derivatives” of particular sequences of nucleic acids refer to nucleic acid sequences that are closely related to a particular sequence but which may possess, either naturally or by design, changes in sequence or structure. By closely related, it is meant that at least about 60%, but often, more than 85%, of the nucleotides of the sequence match over the defined length of the nucleic acid sequence referred to using a specific SEQ ID NO. Changes or differences in nucleotide sequence between closely related nucleic acid sequences may represent nucleotide changes in the sequence that arise during the course of normal replication or duplication in nature of the particular nucleic acid sequence. Other changes may be specifically designed and introduced into the sequence for specific purposes, such as to change an amino acid codon or sequence in a regulatory region of the nucleic acid. Such specific changes may be made in vitro using a variety of mutagenesis techniques or produced in a host organism placed under particular selection conditions that induce or select for the changes. Such sequence variants generated specifically may be referred to as “mutants” or “derivatives” of the original sequence.

The terms “percent similarity”, “percent identity” and “percent homology” when referring to a particular sequence are used as set forth in the University of Wisconsin GCG software program and are known in the art.

The phrase “consisting essentially of” when referring to a particular nucleotide or amino acid means a sequence having the properties of a given SEQ ID NO:. For example, when used in reference to an amino acid sequence, the phrase includes the sequence per se and molecular modifications that would not affect the basic and novel characteristics of the sequence.

A “replicon” is any genetic element, for example, a plasmid, cosmid, bacmid, phage or virus that is capable of replication largely under its own control. A replicon may be either RNA or DNA and may be single or double stranded.

A “vector” is a replicon, such as a plasmid, cosmid, bacmid, phage or virus, to which another genetic sequence or element (either DNA or RNA) may be attached so as to bring about the replication of the attached sequence or element.

An “expression vector” or “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

As used herein, the term “operably linked” refers to a regulatory sequence capable of mediating the expression of a coding sequence, which is placed in a DNA molecule (e.g., an expression vector) in an appropriate position relative to the coding sequence so as to effect expression of the coding sequence. This same definition is sometimes applied to the arrangement of coding sequences and transcription control elements (e.g. promoters, enhancers, and termination elements) in an expression vector. This definition is also sometimes applied to the arrangement of nucleic acid sequences of a first and a second nucleic acid molecule wherein a hybrid nucleic acid molecule is generated.

The term “oligonucleotide,” as used herein refers to a primer and a probe as described herein and is defined as a nucleic acid molecule comprised of two or more ribo- or deoxyribonucleotides, preferably more than three. The exact size of the oligonucleotide will depend on various factors and on the particular application and use of the oligonucleotide.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be “substantially” complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “specifically hybridize” refers to the association between two single-stranded nucleic acid molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Primers and/or probes may be labeled fluorescently with 6-carboxyfluorescein (6-FAM). Alternatively primers may be labeled with 4, 7, 2′, 7′-Tetrachloro-6-carboxyfluorescein (TET). Other alternative DNA labeling methods are known in the art and are contemplated to be within the scope of the invention.

In a particular embodiment, oligonucleotides that hybridize to nucleic acid sequences identified as specific for, for example, any one of SEQ ID NOs: 1-19 as described herein, are at least about 10 nucleotides in length, more preferably at least 15 nucleotides in length, more preferably at least about 20 nucleotides in length. Further to the above, fragments of nucleic acid sequences identified as specific for, for example, any one of SEQ ID NOs: 1-19 described herein represent aspects of the present invention. Such fragments and oligonucleotides specific for same may be used as primers or probes to determining the amount of a P. copri in a biological sample obtained from a subject. Primers such as those described herein, which bind specifically to any one of SEQ ID NOs: 1-19 may, moreover, be used in polymerase chain reaction (PCR) assays in methods directed to determining the amount of P. copri in a biological sample obtained from a subject.

### Kits

Also encompassed herein is a diagnostic pack or kit comprising one or more containers filled with one or more of the diagnostic reagents described herein. Such diagnostic reagents include fragments and oligonucleotides useful in the detection of P. copri (e.g., any one of SEQ ID NOs: 1-19) in a subject or sample isolated therefrom. Diagnostic reagents may comprise a moiety that facilitates detection and/or visualization. Diagnostic reagents may be supplied in solution or immobilized onto a solid phase support. Optionally associated with such container(s) are buffers for performing assays using the diagnostic reagents described herein, negative and positive controls for such assays, and instructional manuals for performing assays.

Unless defined otherwise, all technical and scientific terms used herein have the same meaning as commonly understood by one of ordinary skill in the art to which this invention belongs.

The invention may be better understood by reference to the following non-limiting Examples, which are provided as exemplary of the invention. The following examples are presented in order to more fully illustrate the preferred embodiments of the invention and should in no way be construed, however, as limiting the broad scope of the invention.

All publications mentioned herein are incorporated herein by reference to disclose and describe the methods and/or materials in connection with which the publications are cited.

### Examples

### Materials and Methods

### Study Participants

Consecutive patients from the New York University rheumatology clinics and offices were screened for the presence of RA. After informed consent was signed, each patient's medical history (according to chart review and interview/questionnaire), diet, and medications were determined. A screening musculoskeletal examination and laboratory assessments were also performed or reviewed. All RA patients who met the study criteria were offered enrollment.

### Inclusion and Exclusion Criteria

The criteria for inclusion in the study required that patients meet the American College of Rheumatology/European League Against Rheumatism 2010 classification criteria for RA (Aletaha et al., 2010), including seropositivity for rheumatoid factor (RF) and/or anti-citrullinated protein antibodies (ACPAs) (assessed using an anti-cyclic citrullinated peptide ELISA; Euroimmun), and that all subjects be age 18 years or older. New-onset RA was defined as disease duration of a minimum of 6 weeks and up to 6 months since diagnosis, and absence of any treatment with disease-modifying anti-rheumatic drugs (DMARDs), biologic therapy or steroids (ever). Chronic RA was defined as any patient meeting the criteria for RA whose disease duration was a minimum of 6 months since diagnosis. Most subjects with chronic RA were receiving DMARDs (oral and/or biologic agents) and/or corticosteroids at the time of enrollment. Healthy controls were age-, sex-, and ethnicity-matched individuals with no personal history of inflammatory arthritis.

The exclusion criteria applied to all groups were as follows: recent (<3 months prior) use of any antibiotic therapy, current extreme diet (e.g., parenteral nutrition or macrobiotic diet), known inflammatory bowel disease, known history of malignancy, current consumption of probiotics, any gastrointestinal tract surgery leaving permanent residua (e.g., gastrectomy, bariatric surgery, colectomy), or significant liver, renal, or peptic ulcer disease. This study was approved by the Institutional Review Board of New York University School of Medicine.

### Sample Collection and DNA Extraction

Fecal samples were obtained within 24 h of production. All samples were suspended in MoBio buffer-containing tubes. DNA was extracted using a combination of the MoBio Power Soil kit and a mechanical disruption (bead-beater) method based on a previously described protocol (Ubeda et al., 2010). Samples were stored at −80° C.

V1-V2 16S rDNA Region Amplification and Sequencing

For each sample, 3 replicate PCRs were performed to amplify the V1 and V2 regions as previously described (Ubeda et al., 2010). PCR products were sequenced on a 454 GS FLX Titanium platform (454 Roche) at a depth of at least 2,600 reads per subject. Sequences have been deposited in the NCBI Sequence Read Archive under the accession number SRP023463.

### 16S Sequence Analysis

Sequence data were compiled and processed using MOTHUR (Schloss et al., 2009). Sequences were converted to standard FASTA format. Sequences shorter than 200 bp, containing undetermined bases or homopolymer stretches longer than 8 bp, with no exact match to the forward primer or a barcode, or that did not align with the appropriate 16S rRNA variable region were not included in the analysis. Using the 454 base quality scores, which range from 0-40 (0 being an ambiguous base), sequences were trimmed using a sliding-window technique, such that the minimum average quality score over a window of 50 bases never dropped below 30. Sequences were trimmed from the 3′-end until this criterion was met. Sequences were aligned to the 16S rRNA gene, using as template the SILVA reference alignment (Pruesse et al., 2007), and the Needleman-Wunsch algorithm with the default scoring options. Potentially chimeric sequences were removed using the ChimeraSlayer program (Haas et al., 2011). To minimize the effect of pyrosequencing errors in overestimating microbial diversity (Huse et al., 2010), rare abundance sequences that differ in 1 or 2 nucleotides from a high abundance sequence were merged to the high abundance sequence using the pre.cluster option in MOTHUR. Sequences were grouped into operational taxonomic units (OTUs) using the average neighbor algorithm. Sequences with distance-based similarity of 97% or greater were assigned to the same OTU. OTU-based microbial diversity was estimated by calculating the Shannon diversity index and Simpson Index using mothur. Phylogenetic classification was performed for each sequence using the Bayesian classifier algorithm described by Wang and colleagues with the bootstrap cutoff 60% (Wang et al., 2007).

### Statistical Assessment of Biomarkers Using LEfSe

Briefly, LEfSe pairwise compares abundances of all biomarkers (e.g. bacterial clades) between all groups using the Kruskal-Wallis test, requiring all such tests to be statistically significant. Vectors resulting from the comparison of abundances (e.g. Prevotella relative abundance) between groups are used as input to linear discriminant analysis (LDA), which produces an effect size (FIG. 1a). In analyses performed herein, the main utility of LEfSe over traditional statistical tests is that an effect size is produced in addition to a p- or q-value. This allows us to sort the results of multiple tests by the magnitude of the difference between groups, not only by q-values, as the two are not necessarily correlated. In the case of hierarchically organized groups (e.g. bacterial clades, or KEGG pathways), this lack of correlation can arise from differences in the number of hypotheses considered at different levels in the hierarchy. For example, at the genus level, there may be 1,000 tests performed, requiring a high level of significance to pass multiple testing correction, whereas at the phylum level, only 10 tests may be performed, requiring a less stringent threshold for significance.

### Processing of Illumina Reads

Paired-end reads 100 bp in length were trimmed from both ends to yield the largest contiguous segment where all per-base QVs were >=25. Reads <50 bp in length after this step were discarded. Quality-filtered reads were then aligned to the human reference genome (hg19) using bowtie2 in -very-sensitive-local mode, keeping only those reads that failed to align. Human-filtered reads were then sorted into complete pairs and singletons (whose mates were removed by filtering) for downstream analyses.

Calculation of P. copri DSM 18205 Genome Coverage

The P. copri DSM18205-reference genome (assembly GCA_000157935.1) was first concatenated into a pseudo-contig in order of increasing contig number. Filtered Illumina reads from P. copri positive NORA and healthy (including HMP subjects, Table S2) subjects were aligned to the reference using bowtie2 in -very-sensitive-local mode. Paired-end reads aligning to non-overlapping 1 kb windows across the length of the genome were counted and normalized to FPKM (fragments per kilobase per million reads). The interquartile range (25^{th} to 75^{th} percentile), mean, and median FPKM for each window was calculated and displayed as a boxplot with R.

Generation of a P. copri Pangenome Catalog

Filtered paired-end reads from P. copri positive subjects were first assembled according to the HMP Whole-Metagenome Assembly SOP (Pop, 2011) using SOAPdenovo (Luo et al., 2012). Briefly, paired-end and singleton reads were used concurrently with the parameters -K 25 -R -M 3-d 1. The resulting contigs >300 bp in length were then aligned to the P. copri reference genome with BLASTN at an e-value cutoff of 1e-5. A stringent cutoff requiring at least one hit of 97% identity across 300 bp was used to infer that a contig originated from a strain of P. copri (FIG. S3d). ORFs were then called on the resulting contigs using MetaGeneMark (Zhu et al., 2010). The resulting ORFs were then clustered using USEARCH at an identity threshold of 97% to yield a final set of P. copri genes (FIG. S3b). Samples were excluded from further analyses if they had less than 7 million reads aligning to P. copri (FIG. S3c). This resulted in a catalog of 20,387 putative P. copri ORFs with 9,274+/−1,640 (mean, SD) present in each subject. Further filtering of partially assembled (i.e. containing gaps, lacking stop codons), short (i.e. less than 300 bp), and low-coverage (i.e. present in fewer than five subjects) ORFs yielded a final set of 3,291 high-confidence P. copri ORFs.

Presence or Absence Determination of P. copri Pangenome ORFs

Filtered reads were aligned to the P. copri pangenome catalog using bowtie2 in -very-fast mode. ORFs were said to be present in a sample if at least 97% of their length, minus one read length (i.e. 100 bp) to account for edge alignment artifacts, was covered at an identity of 97% or greater (FIG. S3a).

### Calculation of Differential ORF Presence in Healthy and NORA

The presence or absence of ORFs in each sample was determined as above, and Fisher's exact test was used on 2×2 contingency tables for each ORF. Resulting p-values were adjusted for multiple hypothesis testing by converting to false discovery rate (FDR) q-values using the Benjamini-Hochberg procedure. ORFs with q<0.25 were considered statistically significant. Effect size was calculated using the below equation.

$$Effect Size = \frac{Absent in NORA}{Total Absent} - \frac{Present in NORA}{Total Present}$$

Application of Bayes' Theorem to P. copri Presence and NORA Status

In western cohorts, such as the Human Microbiome Project and present study, the prevalence of P. copri is approximately 19%, i.e. P(Prevotella)=0.19. The approximate incidence of RA is thought to be 1%, i.e. P(NORA)=0.01. In the present cohort, 75% of new-onset RA (NORA) subjects had 5% or more Prevotella OTU4, which the present inventors determined to be P. copri, i.e. P(Prevotella|NORA)=0.75. The present inventors applied Bayes' theorem as given below.

$$P ( NORA \vert Prevotella ) = \frac{P ( Prevotella \vert NORA ) P ( NORA )}{P ( Prevotella )}$$

The solution to this equation gives a 3.95% probability of NORA status if P. copri is present in the gut, compared to a 1% probability of NORA (i.e. the incidence of RA) given no prior information.

### Genome Assembly

Long reads were obtained for several high-Prevotella abundance subjects (028B, 030B, 061B, 089B) on the 454 GS FLX Titanium platform. These reads were assembled with Newbler v2.6 to obtain metagenomic assemblies (Table S1). The resulting contigs were subsequently filtered by alignment to the P. copri DSM 18205 reference genome, keeping those with at least one hit of 97% across 300 bp, to obtain draft patient-derived P. copri genomes.

TABLE S1
Draft genome assembly statistics of four subjects with a high abundance of
Prevotella OTU4.

| | | Prevotella | | Total | | | | P. copri Aligned | | | |
|---|---|---|---|---|---|---|---|---|---|---|---|
| Subject | | OTU4 | | # of | Size | N50 | Mean | # of | Size | N50 | Mean |
| ID | Group | Abundance | # Reads | Contigs | (Mb) | (kb) | Depth | Contigs | (Mb) | (kb) | Depth |
| 028B | NORA | 27.7% | 1,240,515 | 19,988 | 23.24 | 1.45 | 6.13 | 115 | 3.21 | 59.84 | 36.76 |
| 030B | NORA | 50.9% | 1,041,546 | 21,579 | 17.35 | 1.01 | 6.97 | 232 | 2.60 | 16.18 | 44.14 |
| 061B | NORA | 66.5% | 1,209,392 | 9,241 | 12.8 | 1.58 | 9.88 | 74 | 3.23 | 79.98 | 172.64 |
| 089B | NORA | 56.3% | 1,395,872 | 12,112 | 23.47 | 4.64 | 23.12 | 1,963 | 3.96 | 3.19 | 30.39 |
| Ref. Genome | — | — | — | — | — | — | — | 83 | 3.51 | 131.4 | — |

### Statistical Significance of Marker Gene Profiles Between Samplings

If each gene (boxes in FIG. 3b, rows 61 boxes in length) is considered independently and can be in one of two states (i.e. present or absent), the probability of an exact match between any two individuals is 2⁻⁶¹, or 2⁻⁶⁰ with one mismatch. Qualitatively, it can be seen that any intra- or inter-individual comparison is highly statistically significant. Further, if we concede that genes within an island are not truly independent, and there are six such islands which are considered identical with 1-2 mismatches allowed, the probability of such a match is 2⁻⁶, or 0.015625, less than a 0.05 threshold for significance.

Quantification of Metagenome Function with HUMAnN and LEfSe

Filtered paired-end reads were aligned separately to all genomes in KEGG with USEARCH 6.0 (Edgar, 2010) using parameters -usearch_local -maxaccepts 2-maxrejects 8 -evalue 0.1-id 0.80. The results from each read in a pair (and singletons) were combined and processed with HUMAnN 0.96 (Abubucker et al., 2012) with default parameters. Output tables containing per-sample abundance estimates of KEGG modules were then processed with LEfSe (Segata et al., 2011) using an alpha cutoff of 0.001 and an effect size cutoff of 2.0.

### Human Leukocyte Antigen (HLA) Allele Determination

Genomic DNA was isolated from the peripheral blood of RA patients and controls using QIAamp Blood Mini Kit (Qiagen GmbH, Halden, Germany) according to the manufacturer's instructions. HLA-DRB1 alleles were determined by Sequence-Based Typing (SBT) and by Single Specific Primer-Polymerase Chain Reaction (SSP-PCR) methodologies (Fred H Allen Laboratory of Immunogenetics, NY, USA; Weatherall Institute for Molecular Medicine, Oxford, UK) (Table S7). Alleles considered to have the shared-epitope conferring higher risk for RA included: HLA-DRB1*01:01, 01:02, 04:01, 04:04, 04:05, 04:08, 10:01, 13:03, and 14:02, corresponding to S₂ and S_{3P} RA risk classification (du Montcel et al., 2005).

### Colonization of Mice

C57BL/6 or DBA/1 mice (Jackson Laboratories) were treated with ampicillin, neomycin, metronidazole (all 1 g/L) for 7 days prior to gavage. P. copri (CB7, DSMZ) or B. thetaiotamicron (gift from E. Martens) was grown to log phase under anaerobic conditions in PYG liquid media (Anaerobe Systems, CA) and 10⁷ CFU were used to inoculate mice. Feces were collected at 1 and 2 weeks post-gavage to confirm colonization. Fecal DNA was extracted with mechanical bead beating with 0.1 mm zirconia silica beads (Biospecs Inc.) in 2% SDS followed by phenol chloroform extraction. Confirmation of colonization was achieved with P. copri genome specific primers (F: CCGGACTCCTGCCCCTGCAA; SEQ ID NO: 20; R: GTTGCGCCAGGCACTGCGAT; SEQ ID NO: 21); Prevotella 16S primers (F: CACRGTAAACGATGGATGCC; SEQ ID NO: 22; R: GGTCGGGTTGCAGACC; SEQ ID NO: 23), B. thetaiotamicron SusC (F: CACAACAGCCATAGCGTTCCA; SEQ ID NO: 24 R: ATCGCAAAAATAAGATGGGCAAA; SEQ ID NO: 25) (Benjida et al JBC 2011), and Universal 16S Primers (F: ACTCCTACGGGAGGCAGCAGT; SEQ ID NO: 26, R: ATTACCGCGGCTGCTGGC; SEQ ID NO: 27). QPCR was performed with a Roche Lightcycler and the following cycling conditions: 9° C. for 5 m, 40 cycles of 95° C. for 10 s, and 60° C. for 30 s, 72° C. for 30 s. Genomic DNA from P. copri was used to generate a standard curve to quantitate ng of P. copri present per mg of total feces.

### DSS Induced Colitis

Mice were given 2% dextran sulfate sodium (DSS) in drinking water ad libitum for 7 days. Body weight was evaluated every 1-2 days over 14 days. Colonic mucosal damage 0 to 3 cm proximal to the anal verge was evaluated by direct visualization using the Coloview (Karl Storz Veterinary Endoscopy, Tuttlingen, Germany). Endoscopic scoring was performed as previously described: assessment of colon thickening (0-3 points), fibrinization (0-3 points), granularity (0-3 points), morphology of the vascular pattern (0-3 points), and stool consistency (normal to unshaped; 0-3 points) (Becker et al, Nature Protocols 2007).

### Collagen-Induced Arthritis

DBA/1 mice were immunized with complete Freund's adjuvant and type II collagen as previously described (Brand et al., 2007). Briefly, type II chicken collagen (Sigma) was dissolved in dilute acetic acid at 4 mg/mL on ice and mixed 1:1 with CFA to form an emulsion. Mice were immunized intradermally with 50 uL at the base of the tail. Animals were evaluated 2-3 times/week and arthritis score determined based on a severity score of 0-4 every week (Brand et al., 2007).

### Cell Isolation and Intracellular Staining

Lamina propria mononuclear cells were isolated from colonic tissue as previously described (Diehl et al., 2013). Cells were stimulated with phorbol myristate acetate and ionomycin with brefeldin for 4 hours and prepared as per manufacturer's instruction with Cytoperm/Cytofix (BD Biosciences) for intracellular cytokine evaluation of IL-17A (ebiosciences 17B7) and IFNγ (Ebiosciences XMG1.2). For Foxp3 analysis, cells were fixed and permeabilized as per manufacturer's instructions (eBiosciences) and stained intracellularly with anti-Foxp3 (FJK-16 s).

Isolation of P. copri from RA Patient Feces

Feces were collected immediately into anaerobic transport media (Anaerobe Systems). Isolation was performed anaerobically by streaking fecal samples onto plates containing kanamycin and vancomycin (Anaerobe Systems). Colonies were isolated and screened for P. copri by sequencing V3-V5 of the 16S rDNA gene.

Sequencing of Patient P. copri Genomes

DNA was isolated from pure cultures of patient P. copri isolates 622 and 624 (Powersoil Mo Bio Powersoil). Whole genome libraries were prepared with the Nextera DNA Sample Prep Kit (Nextera), and sequenced 2×250 bp on Illumina MiSeq using V2 chemistry (Illumina). Reads were compared to the reference P. copri draft genome (DSM 18205, assembly GCA_000157935.1) using the USEARCH local alignment tool (Edgar, 2010, Bioinformatics 26(19), 2460-2461). Reads were assembled into contigs using Velvet software (Zerbino et al., 2008, Genome Research 18, 821-829). Resulting contigs were compared to the reference P. copri draft genome (DSM 18205, assembly GCA_000157935.1) with progressiveMauve software to generate comparison plots (Darling et al., 2010, PLoS ONE 5, e11147).

### Colonization of Mice

Previously germ free mice were colonized with 10⁶ colony forming units (c.f.u.) of either P. copri reference (DSMZ, CB7), patient P. copri isolate 624, or B. thetaiotaomicron. Colonization was confirmed by qPCR of fecal DNA with P. copri-specific primers, B. thetaiotaomicron SusC primers, and 16S-specific primers.

### Intestinal Cell Isolation and Staining

Lamina propria lymphocytes were isolated from the colon of colonized mice. The epithelium was removed by shaking pieces of tissue for two ten-minute washes in 30 mM EDTA, 10 mM HEPES in PBS at 37 degrees C. After washing in RPMI, tissue was digested for 90 min in complete RPMI containing 100 U/ml type VIII collagenase and 150 ug/ml DNaseI. Lymphocytes were isolated with a Percoll gradient. Cells were stained according the eBioscience protocol for intranuclear reagents using the following antibodies for flow cytometry: CD3 (Alexa Fluor 700), CD4 (PeCy5.5), Rorgt (PE), and Tbet (APC).

### Results

Association of Prevotella with New-Onset Rheumatoid Arthritis

To determine if particular bacterial clades are associated with rheumatoid arthritis, sequencing of the 16S gene (regions V1-V2, 454 platform) was performed on 114 fecal DNA samples—44 samples collected from NORA patients at time of initial diagnosis and prior to immunosuppressive treatment, 26 samples from patients with chronic, treated rheumatoid arthritis (CRA), 16 samples from patients with psoriatic arthritis (PsA), and 28 samples from healthy controls (HLT) (Table 1).

TABLE 1
Demographic and clinical data among subjects with new-onset rheumatoid arthritis (NORA), chronic,
treated rheumatoid arthritis (CRA), psoriatic arthritis (PsA), and healthy controls (HLT).

| | NORA | CRA | PsA | Healthy |
|---|---|---|---|---|
| | (n = 44) | (n = 26) | (n = 16) | (n = 28) |
| Age, years, mean (median) | 42.4 (40.0) | 50.0 (49.0) | 46.3 (46.0) | 42.8 (40.0) |
| Female, % | 75% | 88% | 56% | 75% |
| Disease duration, months, mean (median) | 5.4 (2.0) | 72.3 (48.0) | 0.8 (0.0) | N/A |
| Disease activity parameters | | | | |
| ESR, mm/h, mean | 34.6 | 33.5 | 19.7 | 10.2 |
| CRP, mg/l, mean | 20.6 | 8.2 | 7.6 | 1.1 |
| DAS28, mean (median) | 5.4 (5.7) | 4.7 (5.0) | 4.8 (4.7) | N/A |
| Patient VAS pain, mm, mean (median) | 61.4 (57.5) | 51.5 (62.5) | 50.6 (45.0) | N/A |
| TJC-28, mean (median) | 11.2 (8.5) | 7.6 (7.0) | 8.8 (6.5) | N/A |
| SJC-28, mean (median) | 8.3 (8.0) | 4.6 (3.0) | 4.8 (3.0) | N/A |
| Autoantibody status | | | | |
| IgM-RF positive, % | 95% | 81% | 13% | 11% |
| ACPA positive, % | 100% | 85% | 6% | 7% |
| IgM-RF and/or ACPA positive, % | 100% | 96% | 13% | 14% |
| IgM-RF titer, kU/I, mean (median) | 341.3 (157.0) | 178.2 (89.0) | 3.6 (0.0) | 20.5 (0.0) |
| ACPA titer, kAU/I, mean (median) | 117.6 (114.0) | 90.8 (57.0) | 1.6 (0.0) | 9.6 (0.0) |
| Medication use | | | | |
| Methotrexate, % | 0% | 42% | 6% | 0% |
| Prednisone, % | 0% | 15% | 6% | 0% |
| Biological agent, % | 0% | 12% | 0% | 0% |

Sequences were analyzed with MOTHUR (Schloss et al., 2009) to cluster operational taxonomic units (OTUs, species level classification) at a 97% identity threshold, assign taxonomic identifiers, and calculate clade relative abundances. Although PsA patients revealed a reduction in sample diversity similar to that of IBD patients (Morgan et al., 2012), diversity was comparable between NORA, CRA and healthy groups at 3.02+/−0.66 (mean, SD) overall by Shannon Diversity Index (FIG. S1a). However, when applying Simpson's Dominance Index, the NORA group was less diverse (FIG. S1b), suggesting that these patients harbored a relatively higher abundance of common taxa. Analysis at the major taxonomic hierarchy levels showed no significant differences in either phyla abundance or the ratio of Bacteroidetes/Firmicutes (FIG. S1c) between all groups. At the level of family abundances, however, a significant enrichment of Prevotellaceae in NORA subjects was noted (FIG. 1a, S1d). Using the linear discriminant effect size method (LEfSe, see Methods) (Segata et al., 2011) to compare detected clades (33 families, 177 genera, 996 OTUs) among all groups, the present inventors found a positive association of two specific Prevotella OTUs with NORA and an inverse correlation with Group XIV Clostridia, Lachnospiraceae, and Bacteroides as compared to healthy controls (FIG. 1a). Of all detected Prevotellaceae OTUs, OTU4 was the most highly represented with 171,486 supporting reads at 11.49+/−17.85 (mean, SD) percent of reads per sample. OTU12, the next most abundant Prevotellaceae, was supported by 12,119 reads at 2.00+/−5.42 (mean, SD) percent of reads per sample. Other Prevotellaceae OTUs (including Prevotella OTU934) were more scarcely represented with 1,232+/−2,305 (mean, SD) total supporting reads at less than 0.5% total reads per sample. The present inventors therefore reasoned that OTU4 was the dominant Prevotella in our cohort with 6-fold more supporting reads than the next most abundant OTU. Principal coordinate analysis with Bray-Curtis distances demonstrated that subjects form distinct clusters, irrespective of health or disease status (FIG. 1b). The largest component of microbial variation corresponded to the carriage (or absence) of Prevotella, which significantly differentiated NORA subjects from healthy controls and other forms of arthritis. Consistent with other reports of either high Prevotella or high Bacteroides relative abundance, but rarely a high relative abundance of both, (Faust et al., 2012, Yatsunenko et al., 2012), the present inventors found segregation of Prevotella or Bacteroides dominance in the intestinal microbiome (FIG. 1c).

To taxonomically identify Prevotella OTU4, OTU12, and OTU934, a phylogenetic tree using the consensus 16S sequences of these OTUs and matched regions from known Prevotella taxa was generated (FIG. S2). The analysis revealed these OTUs to cluster tightly with Prevotella copri, a microbe isolated from human feces (Hayashi et al., 2007) and sequenced as part of the HMP's reference genome initiative. To further characterize Prevotella OTU4, the most abundant taxon, the present inventors selected four high-abundance NORA samples (028B, 030B, 061B, and 089B) for shotgun sequencing (single-end, 454 platform). The resulting long reads were used to generate metagenomic assemblies (Table S1, see Methods) which served as input to PhyloPhlAn (Segata et al., 2013). Briefly, PhyloPhlAn locates 400 ubiquitous bacterial genes in a given assembly by sequence alignment in amino acid space, then builds a tree by concatenating the most discriminative positions in each gene into a single long sequence and applying FastTree (Price et al., 2010), a standard tree reconstruction tool. This produced a phylogenomic tree placing the taxon most represented in each sample's metagenomic contigs (i.e. Prevotella OTU4) again in close association with Prevotella copri (FIG. 2a). The present inventors therefore chose to filter the resulting metagenomic assemblies by alignment to the P. copri reference genome to generate draft patient-derived genome assemblies (see Methods). Comparison of these draft assemblies to reference P. copri and to one another revealed a high degree of similarity, with possible genome rearrangements (FIG. 2b).

Overall, 75% (33/44) of the NORA patients and 21.4% (6/28) of the healthy controls carried Prevotella copri in their intestinal microbiota compared to 11.5% (3/26) and 37.5% (6/16) in CRA and PsA patients, respectively, at a threshold for presence of >5% relative abundance. The prevalence of Prevotella copri in NORA compared to CRA, PsA, and healthy controls was statistically significant by chi-squared test, but was not significant in pairwise comparisons of the latter three cohorts (Table S2).

TABLE S2
Statistical comparisons of Prevotella copri
prevalence between cohort groups.

| | | | | Fisher's |
|---|---|---|---|---|
| | Prevalence | Prevalence | Chi-squared | Exact |
| Comparison | #1 | #2 | p-value | p-value |
| ** NORA v. HLT | 33/44 | 6/28 | 2.612e−05 | 1.025e−05 |
| ** NORA v. CRA | 33/44 | 3/26 | 1.031e−06 | 2.551e−07 |
| * NORA v. PsA | 33/44 | 6/16 | 0.01698 | 0.013 |
| HLT v. CRA | 6/28 | 3/26 | 0.5425 | 0.4704 |
| HLT v. PsA | 6/28 | 6/16 | 0.4239 | 0.3032 |
| CRA v. PsA | 3/26 | 6/16 | 0.1087 | 0.06282 |

** p < 0.01
* p < 0.05

Prevotella copri Strains are Variable and Diagnostic

Although initial shotgun sequencing of the patient-derived strains showed their similarity to P. copri, there were notable differences observed in assembled genomes upon comparison with the P. copri reference genome. This observation suggested that the presence or absence of particular genes in these strains might correlate with health or disease phenotypes in this cohort. To address this question, the present inventors performed shotgun sequencing on fecal DNA from NORA and healthy subjects, and chose to compare Prevotella sequences from 18 NORA Prevotella-positive subjects, which allowed for a depth of at least 7 M Prevotella-aligned reads (paired-end, 100 nt, Illumina platform), to those of P. copri from 17 healthy subjects (including 15 from the HMP database and 2 HLT from the cohort) (Table S3). Samples sequenced to a depth of less than 7 M such reads were excluded (FIG. S3c), having insufficient depth for complete recovery of P. copri ORFs (see Methods).

TABLE S3
Read statistics of sequenced samples included in and excluded from
biomarker analyses.

| | | | | | | Excluded from | |
|---|---|---|---|---|---|---|---|
| | | | P. copri | P. copri | | biomarker | Used in |
| Sample ID | Group | Reads | (%) | ORFs | Notes | analysis | HUMAnN |
| Sample_004B_FE | NORA | 34788481 | 0.07 | 51 | | Too few P. copri reads | |
| Sample_005B_FE | NORA | 8355025 | 3.31 | 176 | | Too few P. copri reads | |
| Sample_009B_FE | NORA | 48160461 | 4.92 | 5545 | | Too few P. copri reads | Y |
| Sample_012B_FE | NORA | 106069156 | 0.07 | 87 | | Too few P. copri reads | Y |
| Sample_016B_FE | NORA | 40192399 | 38.01 | 2364 | | Too few P. copri reads | |
| Sample_019B_FE | NORA | 18434488 | 61.69 | 10568 | | | |
| Sample_020B_FE | HLT | 23518211 | 0.0 | NA | | P. copri not detected | Y |
| Sample_027B_FE | NORA | 42932398 | 3.14 | 1470 | | Too few P. copri reads | |
| Sample_028B_FE | NORA | 21330525 | 42.03 | 7769 | Also sequenced on 454 | | Y |
| Sample_030B_FE | NORA | 21225847 | 61.54 | 7729 | Also sequenced on 454 | | |
| Sample_035B_FE | NORA | 22895499 | 0.0 | NA | | P. copri not detected | Y |
| Sample_037B_FE | NORA | 525777 | 64.98 | 514 | | Too few P. copri reads | |
| Sample_040B_FE | NORA | 40648665 | 37.93 | 8755 | | | |
| Sample_041B_FE | NORA | 43309709 | 33.72 | 2069 | | Too few P. copri reads | |
| Sample_049B_FE | NORA | 903955 | 38.92 | 436 | | Too few P. copri reads | |
| Sample_052B_FE | HLT | 10866496 | 31.86 | 6652 | | Too few P. copri reads | |
| Sample_053B_FE | NORA | 48994974 | 42.67 | 8910 | | | |
| Sample_056B_FE | NORA | 18419788 | 40.57 | 8768 | | | |
| Sample_061B_FE | NORA | 53368347 | 76.89 | 8868 | Also sequenced on 454 | | Y |
| Sample_067B_FE | NORA | 11704917 | 49.16 | 7001 | | Too few P. copri reads | |
| Sample_068B_FE | NORA | 22442664 | 53.04 | 10458 | | | Y |
| Sample_069B_FE | HLT | 40190883 | 0.0 | NA | | P. copri not detected | Y |
| Sample_072B_FE | NORA | 172749116 | 57.40 | 11836 | | | Y |
| Sample_078B_FE | NORA | 64962778 | 41.97 | 7452 | | | Y |
| Sample_082B_FE | NORA | 6660155 | 15.99 | 2164 | | Too few P. copri reads | |
| Sample_087B_FE | HLT | 33238347 | 0.0 | NA | | P. copri not detected | Y |
| Sample_088B_FE | NORA | 8620517 | 16.03 | 3306 | | Too few P. copri reads | |
| Sample_089B_FE | NORA | 12284253 | 69.79 | 11241 | Also sequenced on 454 | | |
| Sample_093B_FE | HLT | 58081209 | 43.33 | 8417 | | | Y |
| Sample_100B_FE | HLT | 93039825 | 0.06 | 54 | | Too few P. copri reads | Y |
| Sample_103B_FE | NORA | 50692337 | 8.216 | 6339 | | Too few P. copri reads | Y |
| Sample_104B_FE | NORA | 2619019 | 1.31 | 47 | | Too few P. copri reads | |
| Sample_107B_FE | NORA | 49355267 | 54.93 | 12402 | | | |
| Sample_108B_FE | NORA | 59725359 | 33.54 | 11045 | | | Y |
| Sample_109B_FE | NORA | 63799939 | 34.89 | 12118 | | | Y |
| Sample_126B_FE | NORA | 76812082 | 55.22 | 13064 | | | Y |
| Sample_128B_FE | NORA | 90734299 | 31.67 | 10482 | | | Y |
| Sample_142B_FE | NORA | 155323541 | 31.93 | 7542 | | | Y |
| Sample_144B_FE | NORA | 68053820 | 25.74 | 8173 | | | |
| Sample_150B_FE | HLT | 66502785 | 0.09 | 55 | | Too few P. copri reads | |
| Sample_169B_FE | HLT | 60513831 | 2.34 | 3384 | | Too few P. copri reads | |
| Sample_170B_FE | NORA | 68783324 | 11.74 | 1659 | | Too few P. copri reads | |
| Sample_178B_FE | NORA | 64075166 | 34.39 | 9369 | | | |
| Sample_186B_FE | HLT | 71394171 | 17.94 | 1787 | | Too few P. copri reads | |
| SRS011134 | HLT | 124345431 | 12.10 | 6556 | 158499257, Visit 1 | | Y |
| SRS011271 | HLT | 132460058 | 54.27 | 9661 | 158802708, Visit 1 | | Y |
| SRS011529 | HLT | 132566003 | 43.31 | 9174 | | | Y |
| SRS011586 | HLT | 126027880 | 0.33 | 1601 | | Too few P. copri reads | Y |
| SRS013521 | HLT | 115013916 | 42.37 | 8792 | 159227541, Visit 1 | | Y |
| SRS013687 | HLT | 118239204 | 34.41 | 10171 | 159268001, Visit 1 | | Y |
| SRS015782 | HLT | 106738994 | 7.39 | 6603 | 764224817, Visit 1 | | Y |
| SRS015794 | HLT | 118863364 | 18.23 | 9240 | | | Y |
| SRS017307 | HLT | 126675555 | 62.45 | 9753 | | | Y |
| SRS019582 | HLT | 85374050 | 10.12 | 7297 | | | Y |
| SRS019910 | HLT | 68306917 | 4.61 | 5713 | | Too few P. copri reads | Y |
| SRS022609 | HLT | 122234158 | 32.32 | 7923 | 158499257, Visit 2 | Duplicate; same subject | |
| SRS023526 | HLT | 111858301 | 37.05 | 8988 | 158802708, Visit 2 | Duplicate; same subject | |
| SRS023914 | HLT | 124938155 | 21.13 | 8963 | 159268001, Visit 2 | Duplicate; same subject | |
| SRS024132 | HLT | 109866478 | 2.56 | 4960 | | Too few P. copri reads | Y |
| SRS045713 | HLT | 114972505 | 10.79 | 9239 | | | Y |
| SRS047044 | HLT | 92764420 | 69.10 | 9334 | 764224817, Visit 2 | Duplicate; same subject | |
| SRS049712 | HLT | 124809913 | 68.97 | 9293 | | | Y |
| SRS049959 | HLT | 113707191 | 8.58 | 7681 | | | Y |
| SRS049995 | HLT | 124069684 | 10.80 | 7665 | 159227541, Visit 2 | Duplicate; same subject | |
| SRS050752 | HLT | 104180160 | 27.22 | 7772 | | | Y |
| SRS053398 | HLT | 105657662 | 28.39 | 9123 | | | Y |
| SRS078176 | HLT | 105629277 | 51.03 | 8953 | 159227541, Visit 3 | Duplicate; same subject | |

First, the present inventors examined the coverage of the P. copri reference genome by all subjects, as an indicator of inter-individual strain variability (HMP, 2012). Overall, coverage was similar between healthy and NORA subjects in all but a few regions (FIG. 3a, blue and red horizontal lines). Eight regions were poorly covered in all subjects with mean coverage below the 25^{th} percentile of 0.79 FPKM, while several regions showed substantial variability between individuals (FIG. 3a, gray vertical lines). To determine if the presence or absence of these regions within individuals was consistent between samplings, MetaPhlAn (Segata et al., 2012) was applied to Prevotella-positive HMP samples collected over multiple visits (FIG. 3b). Briefly, MetaPhlAn determines the presence or absence of metagenomic marker genes that are specific to particular bacterial clades by analyzing the coverage of such genes by sequenced reads. Genes are called specific for a bacterial clade if they are not found in any reference genomes outside the clade, but are found in all such genomes within the clade. In concordance with a previous report (Schloissnig et al., 2013) documenting the temporal stability of metagenomic SNP patterns in individuals, the present inventors found that carriage of P. copri genes within an individual varied little between samplings. In addition to a stable set of P. copri core marker genes common to all samples, a subset of variable marker genes was observed to co-occur in islands across the P. copri genome, suggesting genomic rearrangements as a mechanism of variability (FIG. 3a, blue boxes below plot). Together, these results suggest that P. copri strains vary between individuals and retain their individuality over time.

Next, a catalog of P. copri genes present across many individuals (i.e. the P. copri pangenome) was assembled, by performing de novo meta-genome assembly and gene calling on a per-sample basis (see Methods). To determine if any ORFs were differentially present in NORA subjects as compared to healthy controls, the present inventors first reduced the set of interrogated ORFs by filtering partially assembled (i.e. containing gaps, lacking stop codons), short (i.e. less than 300 bp), and low-coverage (i.e. present in fewer than five subjects) ORFs to yield a final set of 3,291 high-confidence P. copri ORFs (FIG. S3). The present inventors found two ORFs differentially present in healthy controls, and 17 ORFs differentially present in NORA (FIG. 3c and Table S4). The two healthy-specific ORFs appear on the same metagenomic contig, encoding a nearly-complete nuo operon for NADH:ubiquinone oxidoreductase (FIG. S4a), adjacent to a Bacteroides conjugative transposon. Similarly, two of the NORA-specific ORFs appear together on another metagenomic contig, encoding an ATP-binding cassette iron transporter (FIG. S4b). These ORFs may represent good biomarkers for discrimination between healthy and disease-associated microbiota in the population at risk for RA. See also FIG. 8.

### Functional Potential of the NORA Metagenome

To determine if the NORA metagenome encodes unique functions compared to healthy subjects, the present inventors applied HUMAnN (Abubucker et al., 2012) to quantitate the coverage and abundances of KEGG (Kanehisa and Goto, 2000) modules (small sets of genes in well-defined metabolic pathways) in healthy controls (n=5) and a representative set of NORA subjects (n=14) with and without Prevotella. The present inventors then applied LEfSe (Segata et al., 2011) to find statistically significant differences between groups. This analysis revealed a low abundance of vitamin metabolism (i.e. biotin, pyroxidal, and folate) and pentose phosphate pathway modules in NORA, consistent with a lack of these functions in Prevotella genomes (FIG. 4). At the coverage level (presence or absence), the NORA metagenome is defined by an absence of functions present in Bacteroides and Clostridia, clades typically found in low abundance in Prevotella-high NORA subjects.

Prevotella and Bacteroides are closely related both functionally and phylogenetically, yet, surprisingly, are rarely found together in high relative abundance despite their ability to dominate the gut microbiome individually (Faust et al., 2012). The present inventors hypothesized that there might be a genetic difference in these two clades that could account for their apparent co-exclusionary relationship. The present inventors therefore sought to find genes differentially present in P. copri but not in any of the most abundant Bacteroides species. This revealed K05919 (superoxide reductase), K00390 (phosphoadenosine phosphosulfate reductase), and several transporters as uniquely present in P. copri (Table S5), and also a set of genes absent in P. copri but present in Bacteroides (Table S6).

Relative Abundance of Prevotella copri in NORA Inversely Correlates with Presence of Shared-Epitope Risk Alleles

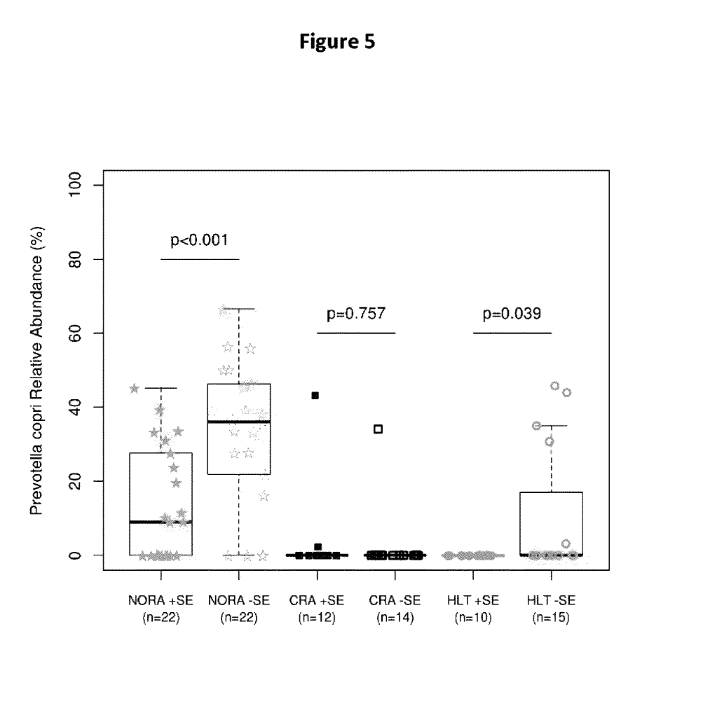

Certain alleles within the human leukocyte-antigen (HLA) Class II locus confer higher risk of disease, in particular those belonging to DRB1 (i.e. “shared epitope” alleles or SE)(du Montcel et al., 2005, Gregersen et al., 1987). To determine whether a higher abundance of P. copri is associated with the host genotype, the present inventors carried out HLA sequencing on DNA from all participants in our study (Table S7). Consistent with recently published mouse data (Gomez et al., 2012), the presence of SE alleles correlated with the composition of the gut microbiota. A subgroup analysis of NORA patients and healthy controls according to presence (or absence) of SE alleles revealed a significantly higher relative abundance of P. copri in those subjects lacking predisposing genes (FIG. 5, p<0.001 in NORA, p<0.05 in HLT, see Methods).

TABLE S7
HLA-DRB1 alleles were determined for subjects in the cohort. Counts
of RA risk alleles (shared epitope) are indicated as 0 for homozygotes
not at risk, 1 for heterozygotes, and 2 for homozygotes at risk
(see Methods). Shared epitope alleles appear in bold.

| | | | | # |
|---|---|---|---|---|
| | | | | Shared |
| | | HLA-DRB1 | HLA-DRB1 | Epitope |
| Sample ID | Group | Allele 1 | Allele 2 | Alleles |
| Sample_008B_FE | CRA | 10:01 | 15:02 | 1 |
| Sample_029B_FE | CRA | 01:02 | 13:04 | 1 |
| Sample_032B_FE | CRA | 03:01/68 | 13:02 | 0 |
| Sample_033B_FE | CRA | 08:04 | 11:01/97/100 | 0 |
| Sample_034B_FE | CRA | 04:07/92 | 14:06 | 0 |
| Sample_036B_FE | CRA | 01:01 | 08:02 | 1 |
| Sample_047B_FE | CRA | 08:03 | 15:01 | 0 |
| Sample_051B_FE | CRA | 14:06 | 16:02 | 0 |
| Sample_071B_FE | CRA | 03:01/68 | 16:02 | 0 |
| Sample_073B_FE | CRA | 10:01 | 13:03 | 2 |
| Sample_075B_FE | CRA | 04:05 | 14:02 | 2 |
| Sample_076B_FE | CRA | 03:02 | 03:02 (*) | 0 |
| Sample_079B_FE | CRA | 11:01/97/100 | 15:01 | 0 |
| Sample_080B_FE | CRA | 08:02 | 15:01 | 0 |
| Sample_084B_FE | CRA | 04:01 | 10:01 | 2 |
| Sample_090B_FE | CRA | 08:01 | 13:02 | 0 |
| Sample_091B_FE | CRA | 15:03 | 15:03 (*) | 0 |
| Sample_092B_FE | CRA | 08:02 | 11:01/97/100 | 0 |
| Sample_094B_FE | CRA | 04:04 | 04:11 | 1 |
| Sample_096B_FE | CRA | 01:01 | 04:01 | 2 |
| Sample_097B_FE | CRA | 04:01 | 04:05 | 2 |
| Sample_102B_FE | CRA | 03:01/68 | 04:01 | 1 |
| Sample_502A_FE | CRA | 04:01 | 04:01 (*) | 2 |
| Sample_504A_FE | CRA | 03:01/68 | 07:01 | 0 |
| Sample_506A_FE | CRA | 04:07/92 | 15:01 | 0 |
| Sample_508A_FE | CRA | 01:01 | 04:01 | 2 |
| Sample_001B_FE | HLT | 11:04 | 13:02 | 0 |
| Sample_003B_FE | HLT | 13:02 | 13:03 | 1 |
| Sample_013B_FE | HLT | NA | NA | NA |
| Sample_014B_FE | HLT | 04:05 | 08:07 | 1 |
| Sample_019B_FE | HLT | 03:01/68 | 13:02 | 0 |
| Sample_020B_FE | HLT | 07:01 | 15:01 | 0 |
| Sample_025B_FE | HLT | 04:02 | 07:01 | 0 |
| Sample_026B_FE | HLT | 07:01 | 15:03 | 0 |
| Sample_045B_FE | HLT | 03:01/68 | 15:03 | 0 |
| Sample_052B_FE | HLT | 07:01 | 14:06 | 0 |
| Sample_064B_FE | HLT | 07:01 | 10:01 | 1 |
| Sample_069B_FE | HLT | NA | NA | NA |
| Sample_070B_FE | HLT | NA | NA | NA |
| Sample_087B_FE | HLT | 04:04 | 14:02 | 2 |
| Sample_093B_FE | HLT | 03:01/68 | 14:54 | 0 |
| Sample_099B_FE | HLT | 07:01 | 13:02 | 0 |
| Sample_100B_FE | HLT | 07:01 | 07:01 (*) | 0 |
| Sample_101B_FE | HLT | 01:01 | 08:01 | 1 |
| Sample_147B_FE | HLT | 03:01/68 | 04:04 | 1 |
| Sample_148B_FE | HLT | 10:01 | 13:03 | 2 |
| Sample_150B_FE | HLT | 04:05 (*) | 11:01 (*) | 1 |
| Sample_169B_FE | HLT | 04:07/92 | 08:02 | 0 |
| Sample_186B_FE | HLT | 03:02 | 11:02 | 0 |
| Sample_187B_FE | HLT | 03:02 | 15:03 | 0 |
| Sample_503A_FE | HLT | 04:01 | 04:01 (*) | 2 |
| Sample_505A_FE | HLT | 03:01/68 | 07:01 | 0 |
| Sample_507A_FE | HLT | 04:07/92 | 15:01 | 0 |
| Sample_509A_FE | HLT | 01:01 | 04:01 | 2 |
| Sample_004B_FE | NORA | 10:01 | 11:01 | 1 |
| Sample_005B_FE | NORA | 04:01 | 04:07/92 | 1 |
| Sample_009B_FE | NORA | 04:11 | 14:02 | 1 |
| Sample_012B_FE | NORA | 04:04:01 | 04:11 | 1 |
| Sample_016B_FE | NORA | 12:02 | 15:02 | 0 |
| Sample_017B_FE | NORA | 04:04 | 04:07/92 | 1 |
| Sample_027B_FE | NORA | 07:01 | 09:01 | 0 |
| Sample_028B_FE | NORA | 04:01 | 14:06 | 1 |
| Sample_030B_FE | NORA | 09:01 | 15:02 | 0 |
| Sample_035B_FE | NORA | 04:07/92 | 14:06 | 0 |
| Sample_037B_FE | NORA | 09:01 | 11:01/97/100 | 0 |
| Sample_038B_FE | NORA | 04:01 | 10:01 | 2 |
| Sample_040B_FE | NORA | 04:01 | 15:03 | 1 |
| Sample_041B_FE | NORA | 04:07/92 | 08:02 | 0 |
| Sample_049B_FE | NORA | 04:05 | 10:01 | 2 |
| Sample_053B_FE | NORA | 08:01 | 10:01 | 1 |
| Sample_056B_FE | NORA | 03:01/68 | 04:11 | 0 |
| Sample_061B_FE | NORA | 03:01/68 | 11:01/97/100 | 0 |
| Sample_067B_FE | NORA | 03:01/68 | 13:02 | 0 |
| Sample_068B_FE | NORA | 07:01 | 11:01/97/100 | 0 |
| Sample_072B_FE | NORA | 04:07/92 | 08:04 | 0 |
| Sample_078B_FE | NORA | 13:03 | 16:02 | 0 |
| Sample_082B_FE | NORA | 04:01 | 04:04 | 2 |
| Sample_088B_FE | NORA | 01:01 | 07:01 | 1 |
| Sample_089B_FE | NORA | 04:101 | 07:01 | 0 |
| Sample_103B_FE | NORA | 04:04 | 15:01 | 1 |
| Sample_104B_FE | NORA | 04:05 | 09:01 | 1 |
| Sample_105B_FE | NORA | 16:02 | 01 (*) | 0 |
| Sample_107B_FE | NORA | 04:05 | 07:01 | 1 |
| Sample_108B_FE | NORA | 01:01 | 15:02 | 1 |
| Sample_109B_FE | NORA | 04:07/92 | 04:03 (*) | 0 |
| Sample_110B_FE | NORA | 03:02 | 07:01 | 0 |
| Sample_112B_FE | NORA | 04:01 | 04:04 | 2 |
| Sample_126B_FE | NORA | 04:11 | 16:02 | 0 |
| Sample_128B_FE | NORA | 04:07/92 | 04:03 (*) | 0 |
| Sample_140B_FE | NORA | 04:07/92 | 09:01 | 0 |
| Sample_142B_FE | NORA | 04:02 | 08:04 | 0 |
| Sample_144B_FE | NORA | 03:02 | 13:04 | 0 |
| Sample_170B_FE | NORA | 04:04 | 15:01 | 1 |
| Sample_172B_FE | NORA | 07:01 | 11:01/97/100 | 0 |
| Sample_174B_FE | NORA | 01:02 | 09:01 | 1 |
| Sample_176B_FE | NORA | 04:05 | 16:02 | 1 |
| Sample_178B_FE | NORA | 03:01/68 | 04:04 | 1 |
| Sample_179B_FE | NORA | 04:01 | 14:54 | 1 |
| Sample_011B_FE | PsA | NA | NA | NA |
| Sample_018B_FE | PsA | 10:01 | 14:02 | 2 |
| Sample_021B_FE | PsA | 03:01/68 | 04:02 | 0 |
| Sample_031B_FE | PsA | 03:01/68 | 04:07/92 | 0 |
| Sample_042B_FE | PsA | 13:01/105/117 | 14:02 | 1 |
| Sample_043B_FE | PsA | 03:01/68 | 10:01 | 1 |
| Sample_055B_FE | PsA | 01:02 | 04:02 | 1 |
| Sample_057B_FE | PsA | 04:04 | 11:01 | 1 |
| Sample_060B_FE | PsA | 07:01 | 15:01 | 0 |
| Sample_062B_FE | PsA | 01:01 | 07:01 | 0 |
| Sample_063B_FE | PsA | 04:06 | 15:01 | 0 |
| Sample_066B_FE | PsA | 04:01 | 07:01 | 1 |
| Sample_077B_FE | PsA | 07:01 | 13:01/105/117 | 0 |
| Sample_085B_FE | PsA | 04:03 | 11:04 | 0 |
| Sample_086B_FE | PsA | 07:01 | 08:02 | 0 |

NA = sample unsuitable for sequencing (e.g. not enough DNA, poor sample quality)
(*) = repeat sequencing (Oxford) after failed initial sequencing (NY Blood Bank)

Prevotella copri Exacerbates Colitis in Mice

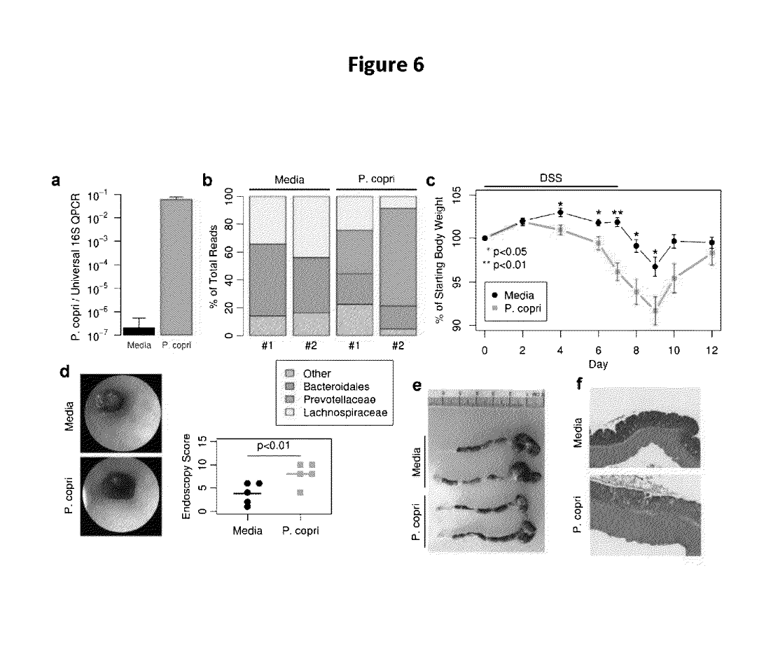

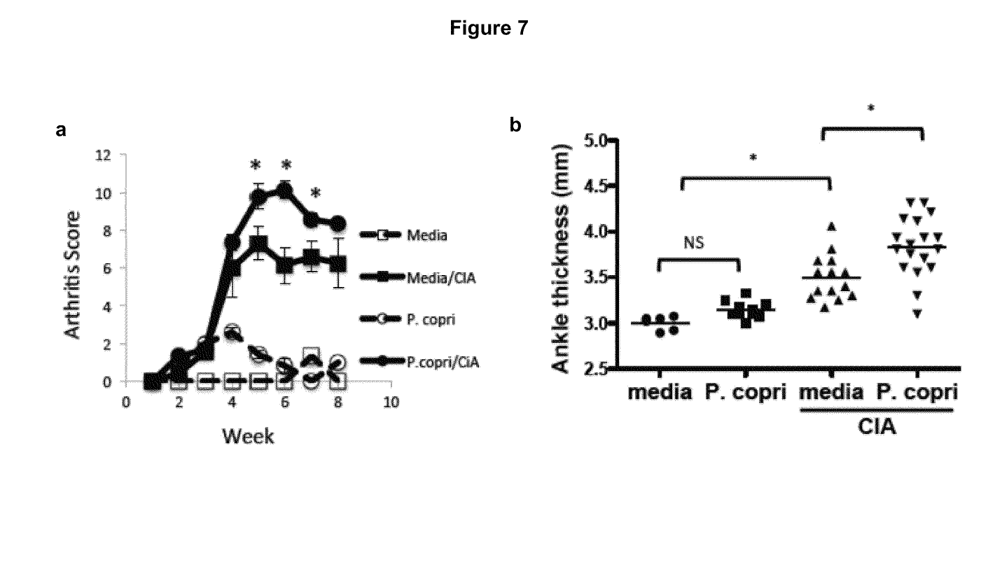

To determine if the Prevotella-associated metagenome is sufficient to predispose to increased inflammatory responses, antibiotic-treated C57BL/6 mice were colonized with P. copri by oral gavage. Analysis of DNA extracted from fecal samples two weeks post-gavage revealed robust colonization with P. copri (FIG. 6a). Sequencing of the 16S gene (regions V1-V2, 454 platform) in fecal DNA from two representative mice colonized with P. copri revealed the ability of Prevotella to dominate the gut microbiota (FIG. 6b). In comparison to fecal DNA from mice gavaged with media alone, P. copri-colonized mice had reduced Bacteroidales and Lachnospiraceae, similar to what was observed in this patient cohort (FIG. 1a, S1d). Consistent with a previous report of a Prevotella taxon exacerbating an inflammatory phenotype (Elinav et al., 2011), exposure of P. copri-colonized mice to 2% dextran sulfate sodium (DSS) in drinking water for 7 days resulted in more severe colitis as assessed by enhanced weight loss (FIG. 6c), worse endoscopic score (FIG. 6d), and increased epithelial damage on histological analysis (FIG. 6e, f) when compared to littermate controls gavaged with media alone. Furthermore, in contrast to mice colonized with mouse commensal Bacteroides thetaiotamicron (FIG. S5a), P. copri colonized mice similarly showed significantly decreased weight loss at day seven following DSS exposure (FIG. S5b). QPCR of DNA extracted from luminal contents of the ileum, cecum, and colon with P. copri-specific primers, moreover, revealed a greater abundance of P. copri in the cecum and colon compared to the ileum when normalized by total 16S or mass of luminal contents (FIG. S6a,b). Analysis of the lamina propria CD4⁺ T-cell response revealed an increase in IFNγ production following DSS induction, although no statistically significant differences were seen in IFNγ (Th1) or IL-17 production (Th17) following P. copri colonization (FIG. S5c). Likewise, no differences in Foxp3⁺ CD4⁺ T-cells were observed. Further to the above, in the collagen-induced arthritis DBA/IJ mouse model colonized with P. copri, P. copri-colonized mice exhibited a statistically significant increase in arthritis scores (FIG. 7a, weeks 5, 6, and 7) as well as ankle thickness (1.51 vs. 1.38 mm, p=0.004; FIG. 7b) following challenge with type II collagen and CFA. These data suggest that a Prevotella-defined microbiome may have the propensity to support inflammation in the context of a genetically susceptible host.

### DISCUSSION

Multiple lines of investigation have revealed that RA is a multifactorial disease that occurs in sequential phases. Notably, there is a prolonged period of autoimmunity (i.e. presence of circulating auto-antibodies such as rheumatoid factor and anti-citrullinated peptide antibodies) in a pre-clinical state that lasts many years, during which time there is no clinical or histologic evidence of inflammatory arthritis (Deane et al., 2010). Before the onset of clinical disease, there is an increase in autoantibody titers and epitope spreading coupled with elevation in circulating pro-inflammatory cytokines. These findings have led to the “second-event” hypothesis in RA, which proposes that an environmental factor triggers systemic joint inflammation in the context of pre-existent autoimmunity. Multiple mucosal sites and their residing microbial communities have been implicated, including the airways, the periodontal tissue and the intestinal lamina propria (McInnes and Schett, 2011, Scher et al., 2012).

Although a role for the gut microbiota has been clearly established in animal models of arthritis, it is not known if dysbiosis influences human RA. The human gut microbiota has been classified into unique enterotypes, one of which is defined by the predominance of Prevotella (Arumugam et al., 2011). In the cohort described herein, the present inventors found the microbiota of many subjects to be defined by a single taxon—Prevotella copri—which was associated with the majority of untreated, new-onset rheumatoid arthritis (NORA) patients. P. copri was also detected in a minority of healthy subjects in cohorts from the Human Microbiome Project (HMP, 2012), the European MetaHIT project (Qin et al., 2010), and the present study. Surprisingly, the frequency of Prevotella copri in chronic rheumatoid arthritis (CRA) patients, all of whom had been treated and exhibited reduced disease activity, was similar to that observed in the healthy subjects. One hypothesis is that the Prevotella-defined microbiota fail to thrive when there is less inflammation, perhaps due to a lack of inflammation-derived terminal electron acceptors, as seen for E. coli in inflammatory bowel disease (Winter et al., 2013). Alternatively, the gut microbiota changes observed in newly diagnosed RA patients may be the consequence of a unique, NORA-specific systemic inflammatory response. While DAS28 scores were slightly lower in CRA and PsA patients (Table 1), the most remarkable difference was in levels of C-reactive protein (CRP). This raises the question of whether CRP itself may have microbial modulating properties. CRP is characteristically high in early and flaring RA, but not in other autoimmune diseases (e.g. systemic lupus erythematous, scleroderma, and PsA). A member of the pentraxin protein family, CRP was first identified in the plasma of patients with Streptococcus pneumoniae infection (Tillett and Francis, 1930). Further, the primary bacterial ligand for CRP is phosphocholine, a component of multiple bacterial cell-wall components, including lipopolysaccharides (LPS). CRP binding to bacterial phosphocholine activates the complement system and enhances phagocytosis by macrophages. Whether or not CRP itself represents a specific response to the presence of P. copri in NORA is an area of future investigation. Interestingly, Prevotella-dominated healthy omnivore individuals were recently reported to have increased basal levels of serum TMAO (trimethylamine N-oxide), a product of inflammation linked to atherogenesis, compared to Bacteroides-dominated healthy individuals (Koeth et al., 2013). While TMAO could be derived from increased consumption of meat (Koeth et al., 2013), Prevotella has been previously associated with a dearth of meat in the diet (Wu et al., 2011). Additional studies are needed to determine if prevalence of P. copri in the microbiota is associated with changes in specific metabolites.

Sequence alignment most closely linked NORA-associated Prevotella with the P. copri genome. Interestingly, large regions of the P. copri genome were scarcely covered in both our cohort and subjects of the HMP. As the reference strain of P. copri was isolated in Japan and all samples analyzed in the present study were collected and sequenced in North America, these differences may reflect geographically-associated strain variability, consistent with a report ranking P. copri as the second-most variable member of the human gut microbiota between continents (Schloissnig et al., 2013). Notably, comparison of sequences in NORA samples with those of P. copri-dominated healthy individuals evaluated in the HMP allowed us to identify ORFs associated with the NORA phenotype. Two ORFs, both encoding components of an iron transporter, were specific for NORA-associated P. copri, while two ORFs were specific for HLT-associated P. copri and encode components of a nuo operon. Iron transporters are known to be virulence factors in other bacterial clades, while the ubiquinone oxidoreductase pathway encoded by the nuo operon may provide a fitness advantage in the context of a healthy microbiome by allowing use of metabolites available therein. While colonization with Prevotella copri increases the pre-test probability of NORA from 1% to approximately 3.95% in western cohorts (by Bayes' theorem, see Methods), the presence of one of the aforementioned ORFs may markedly increase the pre-test probability of NORA status.

Analysis of enzymatic functions in the Prevotella-dominated metagenome reveals a significant decrease in purine metabolic pathways, including tetrahydrofolate (THF) biosynthesis. This may have therapeutic implications since methotrexate (MTX), a folate analogue and a dihydrofolate (DHF) reductase inhibitor, remains the anchor drug for the treatment of RA (Singh et al., 2012) and has inter-individual variability in terms of absorption and bioavailability. The THF biosynthetic pathway encoded by the gut metagenome, which includes a DHF reductase enzyme, may compete with host DHF reductase for MTX binding and metabolism. If so, an increase in DHF reductase-high microbiota in some RA subjects (i.e. Bacteroides overabundant) may help explain, at least partially, why only about half of RA patients respond adequately to oral MTX, ultimately requiring either parenteral administration or the addition of complementary immunosuppressants. Prevotella-high NORA subjects, with a dearth of DHF reductase in the gut, may respond better to oral MTX. Prospective human studies should help to clarify these observations.

RA is a multifactorial autoimmune disease in which certain alleles within the major histocompatibility complex (MHC) class II locus, specifically those belonging to DRB1 (i.e., shared epitope alleles), confer higher risk for disease. A recently published study with HLA-DR transgenic mice revealed that the gut microbiota was, at least partially, regulated by the HLA genes (Gomez et al., 2012). Arthritis-susceptible DRB1*04:01 transgenic mice had a markedly different intestinal microbiota when compared to arthritis-resistant DRB1*04:02 animals, and this was associated with altered mucosal immune function (i.e. increased gene transcripts for Th17-related cytokines) and increased intestinal permeability. Results presented herein suggest that, similarly, SE risk-alleles in humans may have an impact on the composition of the gut microbiota. Intriguingly, patients in the NORA cohort showed a significant inverse correlation between P. copri relative abundance and presence of SE alleles (FIG. 5). It is therefore possible that, as in mice, certain human gut microbial communities are determined by specific MHC alleles that favor the expansion of particular species. As in the case of cigarette smoking, this could also represent a gene-environment interaction that contributes to RA pathogenesis. It is conceivable that a certain threshold for P. copri abundance may be necessary to overcome the lack of genetic predisposition in RA subjects, while a lower abundance may be sufficient to trigger disease in those carrying risk-alleles. Validation in expanded cohorts and mechanistic studies are ongoing to explore further the significance of these findings.

Colonization of mice with P. copri recapitulated the differences in relative abundances of Prevotella and Bacteroides previously reported in humans, and confirmed the ability of P. copri to dominate the colonic commensal microbiota in the absence of apparent disease (Faust et al., 2012). This shift in abundances correlated with a metagenomic shift, which may support and/or perpetuate an inflammatory environment. For example, uniquely present superoxide reductase in P. copri may facilitate resistance to or allow the use of host-derived reactive oxygen species (ROS) generated during inflammation, perhaps as terminal electron acceptors for respiration (Winter et al., 2013). Similarly, the P. copri genome encodes phosphoadenosine phosphosulfate reductase (PAPS), an oxidoreductase absent in Bacteroides that participates in sulfur metabolism and leads to the production of thioredoxin. Intriguingly, thioredoxin has been widely implicated in the pathogenesis of RA and high levels of this redox protein have been found in both serum and synovial fluid of RA patients (Maurice et al., 1999).

Mice colonized with P. copri displayed increased inflammation in DSS-induced colitis. An appealing hypothesis from an evolutionary and ecological perspective is that the P. copri-defined microbiota thrives in a pro-inflammatory environment and may exacerbate inflammation for its own benefit. Another key feature of the P. copri-dominated microbiome is a community shift away from Bacteroides, Group XIV Clostridia, Blautia, and Lachnospiraceae clades, previously reported to be associated with an anti-inflammatory state and regulatory T-cell (Treg) production (Atarashi et al., 2011, Round et al., 2011). This could account, in part, for the observed differences in susceptibility to inflammation (Tao et al., 2011). Further characterization of changes in the host immune system associated with a Prevotella-dominated microbiota should provide deeper insight into the contribution of the expansion of P. copri to the development of autoimmunity in early onset RA.

### REFERENCES

HMP, 2012. Structure, function and diversity of the healthy human microbiome. Nature, 486, 207-14, doi 10.1038/nature11234.
Abdollahi-Roodsaz, S., Joosten, L. A., Koenders, M. I., Devesa, I., Roelofs, M. F., Radstake, T. R., et al. 2008. Stimulation of TLR2 and TLR4 differentially skews the balance of T cells in a mouse model of arthritis. J Clin Invest, 118, 205-16, doi 10.1172/JCI32639.
Abubucker, S., Segata, N., Goll, J., Schubert, A. M., Izard, J., Cantarel, B. L., et al. 2012. Metabolic reconstruction for metagenomic data and its application to the human microbiome. PLoS Comput Biol, 8, e1002358, doi 10.1371/journal.pcbi.1002358.
Aletaha, D., Neogi, T., Silman, A. J., Funovits, J., Felson, D. T., Bingham, C. O., 3rd, et al. 2010. 2010 Rheumatoid arthritis classification criteria: an American College of Rheumatology/European League Against Rheumatism collaborative initiative. Arthritis Rheum, 62, 2569-81, doi 10.1002/art.27584.
Arumugam, M., Raes, J., Pelletier, E., Le Paslier, D., Yamada, T., Mende, D. R., et al. 2011. Enterotypes of the human gut microbiome. Nature, 473, 174-80, doi 10.1038/nature09944.
Atarashi, K., Tanoue, T., Shima, T., Imaoka, A., Kuwahara, T., Momose, Y., et al. 2011. Induction of colonic regulatory T cells by indigenous Clostridium species. Science, 331, 337-41, doi 10.1126/science.1198469.
Deane, K. D., Norris, J. M. & Holers, V. M. 2010. Preclinical rheumatoid arthritis: identification, evaluation, and future directions for investigation. Rheum Dis Clin North Am, 36, 213-41, doi 10.1016/j.rdc.2010.02.001.
Diehl, G. E., Longman, R. S., Zhang, J. X., Breart, B., Galan, C., Cuesta, A., et al. 2013. Microbiota restricts trafficking of bacteria to mesenteric lymph nodes by CX(3)CR1(hi) cells. Nature, 494, 116-20, doi 10.1038/nature11809.
Du Montcel, S. T., Michou, L., Petit-Teixeira, E., Osorio, J., Lemaire, I., Lasbleiz, S., et al. 2005. New classification of HLA-DRB1 alleles supports the shared epitope hypothesis of rheumatoid arthritis susceptibility. Arthritis Rheum, 52, 1063-8, doi 10.1002/art.20989.
Edgar, R. C. 2004. MUSCLE: multiple sequence alignment with high accuracy and high throughput. Nucleic Acids Res, 32, 1792-7, doi 10.1093/nar/gkh340.
Edgar, R. C. 2010. Search and clustering orders of magnitude faster than BLAST. Bioinformatics, 26, 2460-1, doi 10.1093/bioinformatics/btq461.
Elinav, E., Strowig, T., Kau, A. L., Henao-Mejia, J., Thaiss, C. A., Booth, C. J., et al. 2011. NLRP6 inflammasome regulates colonic microbial ecology and risk for colitis. Cell, 145, 745-57, doi 10.1016/j.cell.2011.04.022.
Faust, K., Sathirapongsasuti, J. F., Izard, J., Segata, N., Gevers, D., Raes, J., et al. 2012.
Microbial co-occurrence relationships in the human microbiome. PLoS Comput Biol, 8, e1002606, doi 10.1371/journal.pcbi.1002606.
Frank, D. N., Robertson, C. E., Hamm, C. M., Kpadeh, Z., Zhang, T., Chen, H., et al. 2011. Disease phenotype and genotype are associated with shifts in intestinal-associated microbiota in inflammatory bowel diseases. Inflamm Bowel Dis, 17, 179-84, doi 10.1002/ibd.21339.
Gomez, A., Luckey, D., Yeoman, C. J., Marietta, E. V., Berg Miller, M. E., Murray, J. A., et al. 2012. Loss of sex and age driven differences in the gut microbiome characterize arthritis-susceptible 0401 mice but not arthritis-resistant 0402 mice. PLoS One, 7, e36095, doi 10.1371/journal.pone.0036095.
Gregersen, P. K., Silver, J. & Winchester, R. J. 1987. The shared epitope hypothesis. An approach to understanding the molecular genetics of susceptibility to rheumatoid arthritis. Arthritis Rheum, 30, 1205-13, doi.
Haas, B. J., Gevers, D., Earl, A. M., Feldgarden, M., Ward, D. V., Giannoukos, G., et al. 2011. Chimeric 16S rRNA sequence formation and detection in Sanger and 454-pyrosequenced PCR amplicons. Genome Res, 21, 494-504, doi 10.1101/gr.112730.110.
Hayashi, H., Shibata, K., Sakamoto, M., Tomita, S. & Benno, Y. 2007. Prevotella copri sp. nov. and Prevotella stercorea sp. nov., isolated from human faeces. Int J Syst Evol Microbiol, 57, 941-6, doi 10.1099/ijs.0.64778-0.
Huse, S. M., Welch, D. M., Morrison, H. G. & Sogin, M. L. 2010. Ironing out the wrinkles in the rare biosphere through improved OTU clustering. Environ Microbiol, 12, 1889-98, doi 10.1111/j.1462-2920.2010.02193.x.
Ivanov, Ii, Atarashi, K., Manel, N., Brodie, E. L., Shima, T., Karaoz, U., et al. 2009. Induction of intestinal Th17 cells by segmented filamentous bacteria. Cell, 139, 485-98, doi 10.1016/j.cell.2009.09.033.
Kanehisa, M. & Goto, S. 2000. KEGG: kyoto encyclopedia of genes and genomes. Nucleic Acids Res, 28, 27-30, doi.
Koeth, R. A., Wang, Z., Levison, B. S., Buffa, J. A., Org, E., Sheehy, B. T., et al. 2013. Intestinal microbiota metabolism of 1-carnitine, a nutrient in red meat, promotes atherosclerosis. Nat Med, 19, 576-85, doi 10.1038/nm.3145.
Littman, D. R. & Pamer, E. G. 2011. Role of the commensal microbiota in normal and pathogenic host immune responses. Cell Host Microbe, 10, 311-23, doi 10.1016/j.chom.2011.10.004.
Luo, R., Liu, B., Xie, Y., Li, Z., Huang, W., Yuan, J., et al. 2012. SOAPdenovo2: an empirically improved memory-efficient short-read de novo assembler. Gigascience, 1, 18, doi 10.1186/2047-217X-1-18.
Maurice, M. M., Nakamura, H., Gringhuis, S., Okamoto, T., Yoshida, S., Kullmann, F., et al. 1999. Expression of the thioredoxin-thioredoxin reductase system in the inflamed joints of patients with rheumatoid arthritis. Arthritis Rheum, 42, 2430-9, doi 10.1002/1529-0131(199911) 42:11<2430::AID-ANR22>3.0.CO;2-6.
Mcinnes, I. B. & Schett, G. 2011. The pathogenesis of rheumatoid arthritis. N Engl J Med, 365, 2205-19, doi 10.1056/NEJMra1004965.
Morgan, X. C., Tickle, T. L., Sokol, H., Gevers, D., Devaney, K. L., Ward, D. V., et al. 2012. Dysfunction of the intestinal microbiome in inflammatory bowel disease and treatment. Genome Biol, 13, R79, doi 10.1186/gb-2012-13-9-r79.
Pop, M. 2011. HMP Whole-Metagenome Assembly, http://www.hmpdacc.org/doc/HMP_Assembly_SOP.pdf [Online].
Price, M. N., Dehal, P. S. & Arkin, A. P. 2010. FastTree 2—approximately maximum-likelihood trees for large alignments. PLoS One, 5, e9490, doi 10.1371/journal.pone.0009490.
Pruesse, E., Quast, C., Knittel, K., Fuchs, B. M., Ludwig, W., Peplies, J., et al. 2007. SILVA: a comprehensive online resource for quality checked and aligned ribosomal RNA sequence data compatible with ARB. Nucleic Acids Res, 35, 7188-96, doi 10.1093/nar/gkm864.
Qin, J., Li, R., Raes, J., Arumugam, M., Burgdorf, K. S., Manichanh, C., et al. 2010. A human gut microbial gene catalogue established by metagenomic sequencing. Nature, 464, 59-65, doi 10.1038/nature08821.
Qin, J., Li, Y., Cai, Z., Li, S., Zhu, J., Zhang, F., et al. 2012. A metagenome-wide association study of gut microbiota in type 2 diabetes. Nature, 490, 55-60, doi 10.1038/nature1111450.
Rath, H. C., Herfarth, H. H., Ikeda, J. S., Grenther, W. B., Hamm, T. E., Jr., Balish, E., et al. 1996. Normal luminal bacteria, especially Bacteroides species, mediate chronic colitis, gastritis, and arthritis in HLA-B27/human beta2 microglobulin transgenic rats. J Clin Invest, 98, 945-53, doi 10.1172/JC118878.
Round, J. L., Lee, S. M., Li, J., Tran, G., Jabri, B., Chatila, T. A., et al. 2011. The Toll-like receptor 2 pathway establishes colonization by a commensal of the human microbiota. Science, 332, 974-7, doi 10.1126/science.1206095.
Scher, J. U. & Abramson, S. B. 2011. The microbiome and rheumatoid arthritis. Nat Rev Rheumatol, 7, 569-78, doi 10.1038/nrrheum.2011.121.
Scher, J. U., Ubeda, C., Equinda, M., Khanin, R., Buischi, Y., Viale, A., et al. 2012. Periodontal disease and the oral microbiota in new-onset rheumatoid arthritis. Arthritis Rheum, 64, 3083-94, doi 10.1002/art.34539.
Schloissnig, S., Arumugam, M., Sunagawa, S., Mitreva, M., Tap, J., Zhu, A., et al. 2013. Genomic variation landscape of the human gut microbiome. Nature, 493, 45-50, doi 10.1038/nature11711.
Schloss, P. D., Westcott, S. L., Ryabin, T., Hall, J. R., Hartmann, M., Hollister, E. B., et al. 2009. Introducing mothur: open-source, platform-independent, community-supported software for describing and comparing microbial communities. Appl Environ Microbiol, 75, 7537-41, doi 10.1128/AEM.01541-09.
Sczesnak, A., Segata, N., Qin, X., Gevers, D., Petrosino, J. F., Huttenhower, C., et al. 2011. The genome of th17 cell-inducing segmented filamentous bacteria reveals extensive auxotrophy and adaptations to the intestinal environment. Cell Host Microbe, 10, 260-72, doi 10.1016/j.chom.2011.08.005.
Segata, N., Bornigen, D., Morgan, X. C. & Huttenhower, C. 2013. PhyloPhlAn is a new method for improved phylogenetic and taxonomic placement of microbes. Nat Commun, 4, 2304, doi 10.1038/ncomms3304.
Segata, N., Izard, J., Waldron, L., Gevers, D., Miropolsky, L., Garrett, W. S., et al. 2011. Metagenomic biomarker discovery and explanation. Genome Biol, 12, R60, doi 10.1186/gb-2011-12-6-r60.
Segata, N., Waldron, L., Ballarini, A., Narasimhan, V., Jousson, 0. & Huttenhower, C. 2012. Metagenomic microbial community profiling using unique clade-specific marker genes. Nat Methods, 9, 811-4, doi 10.1038/nmeth.2066.
Singh, J. A., Furst, D. E., Bharat, A., Curtis, J. R., Kavanaugh, A. F., Kremer, J. M., et al. 2012. 2012 update of the 2008 American College of Rheumatology recommendations for the use of disease-modifying antirheumatic drugs and biologic agents in the treatment of rheumatoid arthritis. Arthritis Care Res (Hoboken), 64, 625-39, doi 10.1002/acr.21641.
Stahl, E. A., Raychaudhuri, S., Remmers, E. F., Xie, G., Eyre, S., Thomson, B. P., et al. 2010. Genome-wide association study meta-analysis identifies seven new rheumatoid arthritis risk loci. Nat Genet, 42, 508-14, doi 10.1038/ng.582.
Tao, J., Kamanaka, M., Hao, J., Hao, Z., Jiang, X., Craft, J. E., et al. 2011. IL-10 signaling in CD4+ T cells is critical for the pathogenesis of collagen-induced arthritis. Arthritis Res Ther, 13, R212, doi 10.1186/ar3545.
Tillett, W. S. & Francis, T. 1930. Serological Reactions in Pneumonia with a Non-Protein Somatic Fraction of Pneumococcus. J Exp Med, 52, 561-71, doi.
Ubeda, C., Taur, Y., Jenq, R. R., Equinda, M. J., Son, T., Samstein, M., et al. 2010. Vancomycin-resistant Enterococcus domination of intestinal microbiota is enabled by antibiotic treatment in mice and precedes bloodstream invasion in humans. J Clin Invest, 120, 4332-41, doi 10.1172/JC143918.
Wang, Q., Garrity, G. M., Tiedje, J. M. & Cole, J. R. 2007. Naive Bayesian classifier for rapid assignment of rRNA sequences into the new bacterial taxonomy. Appl Environ Microbiol, 73, 5261-7, doi 10.1128/AEM.00062-07.
Winter, S. E., Winter, M. G., Xavier, M. N., Thiennimitr, P., Poon, V., Keestra, A. M., et al. 2013. Host-derived nitrate boosts growth of E. coli in the inflamed gut. Science, 339, 708-11, doi 10.1126/science.1232467.
Wu, G. D., Chen, J., Hoffmann, C., Bittinger, K., Chen, Y. Y., Keilbaugh, S. A., et al. 2011. Linking long-term dietary patterns with gut microbial enterotypes. Science, 334, 105-8, doi 10.1126/science.1208344.
Wu, H. J., Ivanov, Ii, Darce, J., Hattori, K., Shima, T., Umesaki, Y., et al. 2010. Gut-residing segmented filamentous bacteria drive autoimmune arthritis via T helper 17 cells. Immunity, 32, 815-27, doi 10.1016/j.immuni.2010.06.001.
Yatsunenko, T., Rey, F. E., Manary, M. J., Trehan, I., Dominguez-Bello, M. G., Contreras, M., et al. 2012. Human gut microbiome viewed across age and geography. Nature, 486, 222-7, doi 10.1038/nature11053.
Zanin-Zhorov, A., Ding, Y., Kumari, S., Attur, M., Hippen, K. L., Brown, M., et al. 2010. Protein kinase C-theta mediates negative feedback on regulatory T cell function. Science, 328, 372-6, doi 10.1126/science.1186068.
Zhu, W., Lomsadze, A. & Borodovsky, M. 2010. Ab initio gene identification in metagenomic sequences. Nucleic Acids Res, 38, e132, doi 10.1093/nar/gkq275.

This invention may be embodied in other forms or carried out in other ways without departing from the spirit or essential characteristics thereof. The present disclosure is therefore to be considered as in all aspects illustrate and not restrictive, the scope of the invention being indicated by the appended claims, and all changes which come within the meaning and range of equivalency are intended to be embraced therein.

What is claimed is:

1. A method for determining whether a subject is at risk for developing new onset rheumatoid arthritis (NORA), the method comprising:
isolating a biological sample from the subject;
processing the biological sample to generate a cellular lysate comprising nucleic acid sequences;
analyzing the nucleic acid sequences to measure an amount of at least one NORA marker open reading frame in the cellular lysate, wherein the at least one NORA marker open reading frame is identified in Table S4 and wherein detecting the presence or absence of at least one NORA marker open reading frame in the cellular lysate is correlated with increased risk for developing NORA in the subject.

2. The method of claim 1, wherein the at least one NORA marker open reading frame is a NORA-specific open reading frame and the presence of at least one NORA-specific open reading frame indicates that the subject is at risk for developing NORA, wherein the at least one NORA-specific open reading frame is gene_id_62568 (SEQ ID NO: 1); gene_id_29546 (SEQ ID NO: 2); gene_id_90049 (SEQ ID NO: 3); gene_id_62569 (SEQ ID NO: 4); gene_id_55079 (SEQ ID NO: 5); gene_id_83051 (SEQ ID NO: 6); gene_id_79069 (SEQ ID NO: 7); gene_id_68986 (SEQ ID NO: 8); gene_id_54057 (SEQ ID NO: 9); gene_id_45456 (SEQ ID NO: 10); gene_id_29407 (SEQ ID NO: 11); gene_id_45366 (SEQ ID NO: 12); gene_id 81143 (SEQ ID NO: 13); gene_id 45134 (SEQ ID NO: 14); gene_id_17194 (SEQ ID NO: 15); gene_id_68779 (SEQ ID NO: 16); or gene_id 59356 (SEQ ID NO: 17).

3. The method of claim 2, wherein the presence of increasing numbers of the NORA-specific open reading frames in the subject is directly correlated with greater risk for developing NORA.

4. The method of claim 1, wherein the at least one NORA marker open reading frame is a healthy-specific open reading frame and the absence of at least one healthy-specific open reading frame indicates that the subject is at risk for developing NORA, wherein the at least one healthy-specific open reading frame is gene_id_3694 (SEQ ID NO: 18) or gene_id_3690 (SEQ ID NO: 19).

5. The method of claim 1, wherein the at least one NORA marker open reading frame is a healthy-specific open reading frame and the presence of at least one healthy-specific open reading frame indicates that the subject is at reduced risk for developing NORA, wherein the at least one healthy-specific open reading frame is gene_id_3694 (SEQ ID NO: 18) or gene_id 3690 (SEQ ID NO: 19).

6. The method of claim 1, wherein the subject is selected for evaluation because the subject has a familial history of rheumatoid arthritis (RA) and/or exhibits at least one of the seven diagnostic criteria recognized by The American Rheumatism Association to diagnose RA.

7. The method of claim 1, wherein the biological sample is fecal material, biopsies of specific organ tissues, including large and small intestinal biopsies, synovial fluid, and synovial fluid biopsies.

8. The method of claim 1, further comprising assessment of familial history of RA in the subject, clinical symptoms of RA, ACPA/RF levels, or Th17/Treg levels in the subject.

9. The method of claim 1, wherein the presence or absence of the at least one NORA marker open reading frame in the biological sample is determined by nucleic acid sequencing.

10. The method of claim 9, wherein the nucleic acid sequencing is shotgun sequencing.

11. The method of claim 1, wherein the presence or absence of the at least one NORA marker open reading frame is determined using a reagent that specifically binds to the at least one NORA marker open reading frame or a protein encoded thereby.

12. The method of claim 11, wherein the reagent is selected from the group consisting of an antibody, an antibody derivative, an antibody fragment, a nucleic acid probe, an oligonucleotide, and an oligonucleotide primer pair specific for any one of SEQ ID NOs: 1-19.

13. The method of claim 11, wherein determining the presence or absence of the at least one NORA indicator open reading frame or protein encoded thereby includes at least one assay selected from the group consisting of nucleic acid sequencing, PCR amplification, a competitive binding assay, a non-competitive binding assay, a radioimmunoassay, immunohistochemistry, an enzyme-linked immunosorbent assay (ELISA), a sandwich assay, a gel diffusion immunodiffusion assay, an agglutination assay, dot blotting, a fluorescent immunoassay such as fluorescence-activated cell sorting (FACS), a chemiluminescence immunoassay, an immunoPCT immunoassay, a protein A or protein G immunoassay, and an immunoelectrophoresis assay.

14. A method for evaluating therapeutic efficacy of an agent administered to a patient with RA, the method comprising:
isolating a biological sample from the patient with RA before and after administering the agent;
processing each of the biological samples to generate a cellular lysate comprising nucleic acid sequences of each of the biological samples;
analyzing the nucleic acid sequences of each of the biological samples to measure an amount of at least one of SEQ ID NOs: 1-19 before administration of the agent and an amount of least one of SEQ ID NOs: 1-19 after administration of the agent; and
comparing the amount of the least one of SEQ ID NOs: 1-19 determined before and after administration of the agent, wherein a decrease in the amount of at least one of SEQ ID NOs: 1-17 and/or an increase in the amount of at least one of SEQ ID NO: 18 or SEQ ID NO: 19 after administration of the agent is a positive indicator of the therapeutic efficacy of the agent for RA.

15. The method of claim 14, further comprising assessment of clinical symptoms of RA, ACPA/RF levels, or Th17/Treg levels in the patient with RA.

16. A method for identifying a test substance that modulates levels of Prevotella copri in a subject, said method comprising a) isolating a biological sample from the subject and determining the amount of the at least one of SEQ ID NOs: 1-19 in the biological sample obtained from said subject; b) contacting the biological sample with a test substance; and c) determining the amount of the at least one of SEQ ID NOs: 1-19 in the biological sample after contacting with the test substance, wherein an alteration in the amount of the at least one of SEQ ID NOs: 1-19 determined in step c) relative to the amount determined in step a) identifies the test substance as a modulator of Prevotella copri levels.

17. The method of claim 16, wherein a decrease in the amount of the at least one of SEQ ID NOs: 1-17 determined in step c) when compared to the amount of the at least one of SEQ ID NOs: 1-17, respectively, determined in step a) indicates that the test substance is a potential agent for treating or preventing RA in a subject.

18. The method of claim 16, wherein an increase in the amount of the at least one of SEQ ID NOs: 18 or 19 determined in step c) when compared to the amount of the at least one of SEQ ID NOs: 18 or 19, respectively, determined in step a) indicates that the test substance is a potential agent for treating or preventing RA in a subject.

19. A composition for predicting risk for developing NORA or prognosis of a NORA patient undergoing a therapeutic regimen, the composition comprising specific detection reagents for determining the presence or absence of at least one of SEQ ID NOs: 1-19 of claim 1 and a buffer compatible with the activity of the specific detection reagents.

20. The composition of claim 19, wherein the specific detection reagents comprise a nucleic acid probe, an oligonucleotide, or an oligonucleotide primer pair specific for the at least one of SEQ ID NOs: 1-19.

21. The composition of claim 19, wherein the specific detection reagents are labeled with a detectable moiety.

22. The composition of claim 19, wherein the specific detection reagents are immobilized on a solid phase support.
